(19) United States
(12) Patent Application Publication
(10) Pub. No.: US 2010/0197679 A1
(43) Pub. Date: Aug. 5, 2010

(54) COMPOUNDS

(75) Inventors: Christopher Barfoot, Hertfordshire (GB); David Thomas Davies, Hertfordshire (GB); Timothy James Miles, Hertfordshire (GB); Neil David Pearson, Collegeville, PA (US)

Correspondence Address: GlaxoSmithKline;GLOBAL PATENTS -US, UW2220, P. O. BOX 1539, KING OF PRUSSIA, PA, 19406-0939, US

(73) Assignee: Glaxo Group Limited

(21) Appl. No.: 12/596,701

(22) PCT Filed: Apr. 17, 2008
(86) PCT No.: PCT/EP08/54666

§ 371 Date: Mar. 11, 2010

(30) Foreign Application Priority Data
Apr. 20, 2007 (GB) 0707704.3

(51) Int. Cl. A61K 31/436; C07D 491/056; C07D 498/04; A61K 31/4985; A61K 31/5383; A61P 31/04

(52) U.S. Cl. 514/230.5; 546/115; 544/350; 544/235; 544/105; 514/302; 514/249

(57) ABSTRACT

Bicyclic nitrogen containing compounds and their use as antibacterials

This invention relates to novel compounds, compositions containing them and their use as antibacterials including use in the treatment of tuberculosis.

WO02/08224, WO02/50061, WO02/56882, WO02/96907, WO2003087098, WO2003010138, WO2003064421, WO2003064431, WO2004002992, WO2004002490, WO2004014361, WO2004041210, WO2004096982, WO2002050036, WO2004058144, WO2004087145, WO06002047, WO06014580, WO06010040, WO06017326, WO06012396, WO06017468, WO06020561, WO2006081179, WO2006081264, WO2006081289, WO2006081178, WO2006081182, WO01/25227, WO02/40474, WO02/07572, WO2004035569, WO2004089947, WO04024712, WO04024713, WO04087647, WO2005016916, WO2005097781, WO06010831, WO06021448, WO06032466, WO06038172, WO06046552, WO06099884, WO06126171, WO06105289, WO06125974, WO06134378, WO06137485, WO07016610, WO07081597, WO07071936, WO07115947, WO07118130, WO07122258, WO08006648, WO08003690 and WO08009700 disclose quinoline, naphthyridine, morpholine, cyclohexane, piperidine and piperazine derivatives having antibacterial activity. WO2006044405 discloses arylsulfonamides as EP3 receptor antagonists. WO2003084948 discloses nitrogenous heterocyclic compounds as sodium channel blockers.

This invention provides a compound of formula (I) or a pharmaceutically acceptable salt or N-oxide thereof:

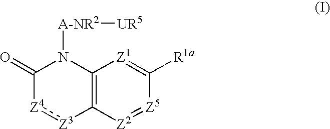

wherein:
Z⁴ is CH and two of Z¹, Z² and Z³ are independently CR^{1b} or N and the remainder are independently CR^{1b}, with a double bond between Z³ and Z⁴;
or one of Z¹ and Z² is CR^{1b} or N and the other is independently CR^{1b}, Z³ is O and Z⁴ is CH₂;
Z⁵ is CH or CF when Z² is CR^{1b}, or CH when Z² is N;
R^{1a} and R^{1b} are independently selected from hydrogen; halogen; cyano; nitro; (C₁₋₆)alkyl; (C₁₋₆)alkylthio; mono-, di- or tri-fluoromethyl; mono-, di- or tri-fluoromethoxy; carboxy; (C₁₋₆)alkoxycarbonyl; hydroxy optionally substituted with (C₁₋₆)alkyl or (C₁₋₆)alkoxy-substituted(C₁₋₆)alkyl; (C₁₋₆)alkoxy-substituted(C₁₋₆)alkyl; hydroxy (C₁₋₆)alkyl; an amino group optionally N-substituted by one or two (C₁₋₆)alkyl, formyl, (C₁₋₆)alkylcarbonyl or (C₁₋₆)alkylsulphonyl groups; and aminocarbonyl wherein the amino group is optionally substituted by one or two (C₁₋₄)alkyl; or R^{1a} and R^{1b} at Z¹ may together form an ethylenedioxy group;
or when one of Z² and Z³ is CR^{1b}, R^{1b} may instead be:
(C₃₋₆)cycloalkyl; (C₃₋₆)cycloalkoxy; (C₂₋₆)alkenyl optionally substituted by carboxy, (C₁₋₆)alkoxycarbonyl or aminocarbonyl wherein the amino group is optionally substituted by one or two (C₁₋₄)alkyl; (C₁₋₆)alkylcarbonyl; (C₁₋₆)alkylcarbonyl oxime; (C₁₋₄)alkyloxycarbonyl(C₁₋₆)alkyloxy; (C₁₋₄)alkylaminocarbonyl(C₁₋₆)alkyloxy; amino substituted by (C₁₋₄)alkylaminocarbonyl; aminocarbonyl wherein the amino group is substituted by (C₁₋₄)alkoxysulphonyl, hydroxy(C₁₋₄)alkyl, (C₁₋₄)alkoxy-substituted(C₁₋)alkyl, (C₃₋₆)cycloalkyl, phenyl, benzyl, monocyclic heteroaryl or monocyclic heteroaryl-methyl; benzyloxy; phenyl; benzyl; monocyclic heteroaryl; or monocyclic heteroaryl-methyl;
wherein heteroaryl is a 5 or 6 membered ring containing up to four hetero-atoms selected from oxygen, nitrogen and sulphur, and wherein a heteroaryl or phenyl ring in R^{1b} may be optionally C-substituted by up to three groups selected from (C₁₋₄)alkylthio; halo; carboxy(C₁₋₄)alkyl; halo(C₁₋₄)alkoxy; halo(C₁₋₄)alkyl; (C₁₋₄)alkyl; (C₂₋₄)alkenyl; (C₁₋₄)alkoxycarbonyl; formyl; (C₁₋₄)alkylcarbonyl; (C₂₋₄)alkenyloxycarbonyl; (C₂₋₄)alkenylcarbonyl; (C₁₋₄)alkylcarbonyloxy; (C₁₋₄)alkoxycarbonyl(C₁₋₄)alkyl; hydroxy; hydroxy(C₁₋₄)alkyl; mercapto(C₁₋₄)alkyl; (C₁₋₄)alkoxy; nitro; cyano; carboxy; amino or aminocarbonyl optionally substituted by one or two (C₁₋₄)alkyl; (C₁₋₄)alkylsulphonyl; (C₂₋₄)alkenylsulphonyl; or aminosulphonyl wherein the amino group is optionally substituted by (C₁₋₄)alkyl or (C₂₋₄)alkenyl;
R² is hydrogen, or (C₁₋₄)alkyl;
A is a group (i) or (ii):

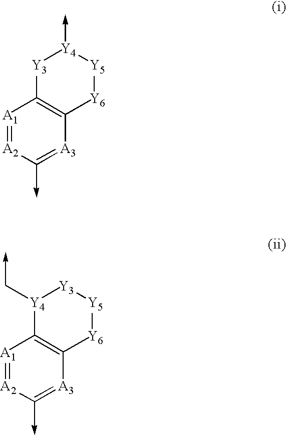

A₁, A₂ and A₃ are independently N or CR³; or
A₃ is N and A₁ and A₂ together form O, S, or NR⁴;
Y₃, Y₅ and Y₆ are independently CHR³, CO or X;

### Y₄ is CR³;

### X is NR⁴ or O;

provided that no more than one group Y₃, Y₅ and Y₆ is X and no more than one group

### Y₃, Y₅ and Y₆ is CO;

and provided that A is optionally substituted by up to two groups R³;
R³ is as defined for R^{1a} or is carboxy(C₁₋₄)alkyl or amino(C₁₋₄)alkyl where the amino group is optionally N-substituted by one or two (C₁₋₄)alkyl or (C₁₋₄)alkylcarbonyl groups;

R⁴ is hydrogen; methyl; carboxy(C₁₋₄)alkyl; (C₂₋₄)alkyl optionally substituted with hydroxy, (C₁₋₄)alkoxy or amino wherein the amino group is optionally substituted by one or two (C₁₋₄)alkyl, (C₁₋₄)alkoxycarbonyl (C₁₋₄)alkylcarbonyl or (C₁₋₄)alkylsulphonyl groups; wherein any alkyl group in R⁴ is optionally substituted with 1-3 fluorine atoms;

U is selected from CO, and CH₂ and
R⁵ is an optionally substituted bicyclic carbocyclic or heterocyclic ring system (B):

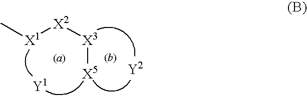

containing up to four heteroatoms in each ring in which
at least one of rings (a) and (b) is aromatic;

X¹ is C or N when part of an aromatic ring, or CR¹⁴ when part of a non-aromatic ring;

X² is N, NR¹³, O, S(O)ₓ, CO or CR¹⁴ when part of an aromatic or non-aromatic ring or may in addition be CR¹⁴R¹⁵ when part of a non aromatic ring;

X³ and X⁵ are independently N or C;

Y¹ is a 0 to 4 atom linker group each atom of which is independently selected from N, NR¹³, O, S(O)ₓ, CO and CR¹⁴ when part of an aromatic or non-aromatic ring or may additionally be CR¹⁴R¹⁵ when part of a non aromatic ring;

Y² is a 2 to 6 atom linker group, each atom of Y² being independently selected from N, NR¹³, O, S(O)ₓ, CO, CR¹⁴ when part of an aromatic or non-aromatic ring or may additionally be CR¹⁴R¹⁵ when part of a non aromatic ring;

each of R¹⁴ and R¹⁵ is independently selected from: H; (C₁₋₄)alkylthio; halo; carboxy(C₁₋₄)alkyl; (C₁₋₄)alkyl; (C₁₋₄)alkoxycarbonyl; (C₁₋₄)alkylcarbonyl; (C₁₋₄)alkoxy (C₁₋₄)alkyl; hydroxy; hydroxy(C₁₋₄)alkyl; (C₁₋₄)alkoxy; nitro; cyano; carboxy; amino or aminocarbonyl optionally mono- or di-substituted by (C₁₋₄)alkyl; or

R¹⁴ and R¹⁵ may together represent oxo;

each R¹³ is independently H; trifluoromethyl; (C₁₋₄)alkyl optionally substituted by hydroxy, (C₁₋₆)alkoxy, (C₁₋₆)alkylthio, halo or trifluoromethyl; (C₂₋₄)alkenyl; (C₁₋₄)alkoxycarbonyl; (C₁₋₄)alkylcarbonyl; (C₁₋₆)alkylsulphonyl; aminocarbonyl wherein the amino group is optionally mono or disubstituted by (C₁₋₄)alkyl; each x is independently 0, 1 or 2.

This invention also provides a method of treatment of bacterial infections including tuberculosis in mammals, particularly in man, which method comprises the administration to a mammal in need of such treatment an effective amount of a compound of formula (I), or a pharmaceutically acceptable salt or N-oxide thereof.

The invention also provides the use of a compound of formula (I), or a pharmaceutically acceptable salt or N-oxide thereof, in the manufacture of a medicament for use in the treatment of bacterial infections including tuberculosis in mammals.

The invention also provides a pharmaceutical composition comprising a compound of formula (I), or a pharmaceutically acceptable salt or N-oxide thereof, and a pharmaceutically acceptable carrier.

In one particular aspect, Y₃, Y₅ and Y₆ are CH₂ and Y₄ is CH:

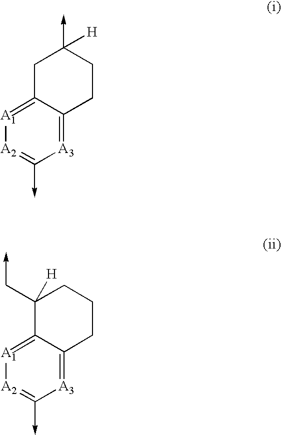

In another particular aspect, Y₃ and Y₅ are CH₂, Y₄ is CH and Y₆ is X:

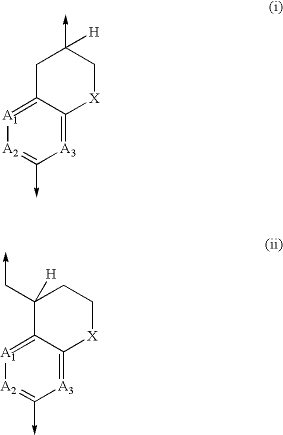

In another particular aspect, A is a group (i) and Y₃ is CH₂, Y₄ is CH, Y₅ is CO and Y₆ is NO:

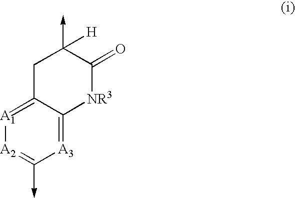

In another particular aspect, A is group (ii), Y₄ is CH, Y₆ is CH₂ and one of Y₃ or Y₅ is X and the other is CH₂.

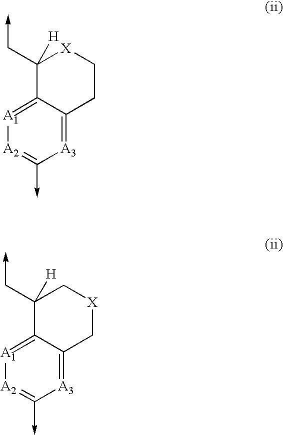

In further aspects:

(1) A¹, A² and A³ are each CR³;
(2) A¹ is N and A² and A³ are each CR³;
(3) A³ is N and A¹ and A² together are S: or
(4) A¹ is CR³ and A² and A³ are each N.

In particular aspects:

(1) Z⁴ is CH and each of Z¹, Z² and Z³ is independently CR^{1b};
(2) Z⁴ is CH and Z¹ is N and Z² and Z³ are independently CR^{1b};

### (3) Z⁴ is CH and Z¹ and Z³ are N and Z² is CR^{1b};

(4) Z⁴ is CH and Z³ is N and Z¹ and Z² are independently CR^{1b};
(5) Z⁴ is CH₂, Z³ is O and Z¹ and Z² are independently CR^{1b}; or

### (6) Z⁴ is CH, Z² and Z³ are N and Z¹ is CR^{1b}.

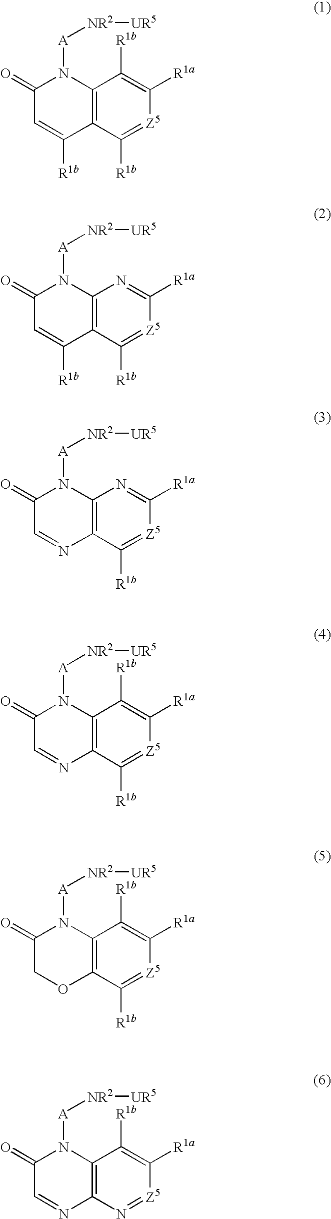

In a particular aspect each R^{1a} and R^{1b} is independently hydrogen, (C₁₋₄)alkoxy, (C₁₋₄)alkylthio, (C₁₋₄)alkyl, cyano, carboxy, hydroxymethyl or halogen; more particularly hydrogen, methoxy, methyl, ethyl, cyano, or halogen. Particular R^{1b} substituents at Z² or Z³ are methyl, cyano or CH═CHCO₂H.

In some embodiments only one group R^{1a} and R^{1b} is other than hydrogen. In a particular embodiment R^{1a} is methoxy, cyano or halo such as fluoro, chloro or bromo and R^{1b} is hydrogen.

In other embodiments two groups R^{1a} and R^{1b} are other than hydrogen. In particular R^{1a} is fluoro and R^{1b} is other than hydrogen, for example fluoro, ethyl or methoxy.

In other embodiments when one of Z² and Z³ is CR^{1b}, R^{1b} is selected from cyano, methyl and CH═CHCO₂H.

In particular aspects, the invention provides compounds of formulae (IA), (IB) and (C):

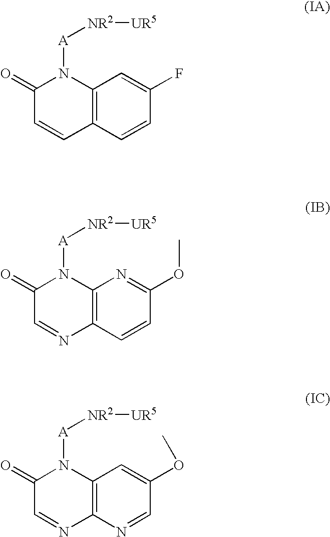

In a particular aspect R² is hydrogen.

Particular examples of R³ include hydrogen; optionally substituted hydroxy; optionally substituted amino; halogen; (C₁₋₄) alkyl; 1-hydroxy-(C₁₋₄) alkyl; optionally substituted aminocarbonyl. More particular R³ groups are hydrogen; CONH₂; 1-hydroxyalkyl e.g. CH₂OH; optionally substituted hydroxy e.g. methoxy; optionally substituted amino; and halogen, in particular fluoro. Most particularly R³ is hydrogen or hydroxy.

In a particular aspect, no or only one R³ group is other than hydrogen.

In a particular aspect R⁴ in NR⁴ formed by A₁ and A₂ is hydrogen or methyl.

More particularly A is a group selected from:

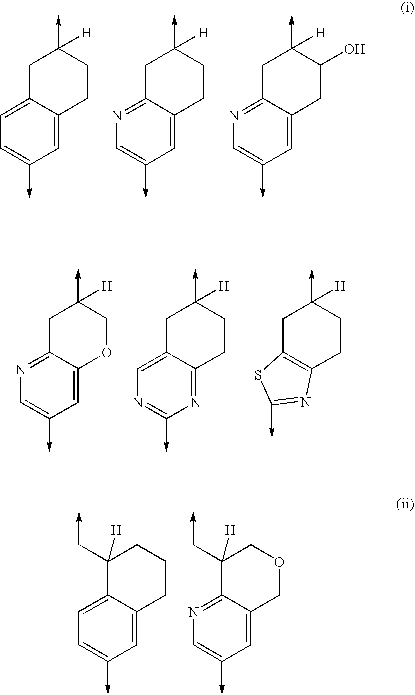

Yet more particularly A is a group selected from:

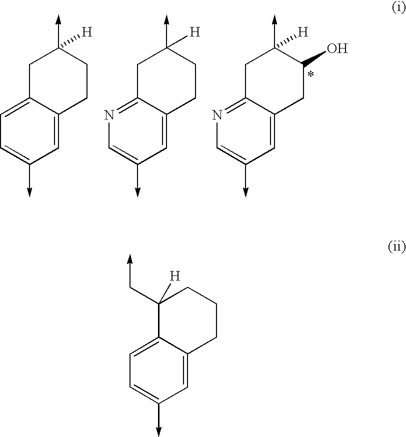

*relative stereochemistry, includes either or both cis diastereomers

In certain embodiments U is CH₂.

In certain embodiments R⁵ is an aromatic heterocyclic ring (B) having 8-11 ring atoms including 2-4 heteroatoms of which at least one is N or NR¹³ in which, in particular embodiments, Y² contains 2-3 heteroatoms, one of which is S and 1-2 are N, with one N bonded to X³.

In alternative embodiments the heterocyclic ring (B) has ring (a) aromatic selected from optionally substituted benzo, pyrido, pyridazino and pyrimidino and ring (b) non aromatic and Y² has 3-4 atoms including at least one heteroatom, with O, S, CH₂ or NR¹³ bonded to X⁵, where R¹³ is other than hydrogen, and either NHCO bonded via N to X³, or O, S, CH₂, or NH bonded to X³. In a particular aspect the ring (a) contains aromatic nitrogen, and more particularly ring (a) is pyridine. Examples of rings (B) include optionally substituted:

(a) and (b) aromatic
1H-pyrrolo[2,3-b]-pyridin-2-yl,
1H-pyrrolo[3,2-b]-pyridin-2-yl,
3H-imidazo[4,5-b]-pyrid-2-yl,
3H-quinazolin-4-one-2-yl,
benzimidazol-2-yl,
benzo[1,2,3]-thiadiazol-5-yl,
benzo[1,2,5]-oxadiazol-5-yl,
benzofur-2-yl,
benzothiazol-2-yl,
benzo[b]thiophen-2-yl,
benzoxazol-2-yl,
chromen-4-one-3-yl,
imidazo[1,2-a]pyridin-2-yl,
imidazo-[1,2-a]-pyrimidin-2-yl,
indol-2-yl,
indol-6-yl,
isoquinolin-3-yl,
[1,8]-naphthyridine-3-yl,
oxazolo[4,5-b]-pyridin-2-yl,
quinolin-2-yl,
quinolin-3-yl,
quinoxalin-2-yl,
naphthalen-2-yl
1,3-dioxo-isoindol-2yl,
1H-benzotriazol-5-yl,
1H-indol-5-yl,
3H-benzooxazol-2-one-6-yl,
3H-benzooxazol-2-thione-6-yl,
3H-benzothiazol-2-one-5-yl,
3H-quinazolin-4-one-6-yl,
benzo[1,2,3]thiadiazol-6-yl,
benzo[1,2,5]thiadiazol-5-yl,
benzo[1,4]oxazin-2-one-3-yl,
benzothiazol-5-yl,
benzothiazol-6-yl,
cinnolin-3-yl,
imidazo[1,2-b]pyridazin-2-yl,
pyrazolo[1,5-a]pyrazin-2-yl,
pyrazolo[1,5-a]pyridin-2-yl,
pyrazolo[1,5-a]pyrimidin-6-yl,
pyrazolo[5,1-c][1,2,4]triazin-3-yl,
pyrido[1,2-a]pyrimdin-4-one-2-yl,
pyrido[1,2-a]pyrimidin-4-one-3-yl,
quinazolin-2-yl,
quinoxalin-6-yl,
thiazolo[3,2-a]pyrimidin-5-one-7-yl,
thiazolo[5,4-b]pyridin-2-yl,
thieno[3,2-b]pyridin-6-yl,
thiazolo[5,4-b]pyridin-6-yl,
thiazolo[4,5-b]pyridin-5-yl,
[1,2,3]thiadiazolo[5,4-b]pyridin-6-yl,
2H-isoquinolin-1-one-3-yl

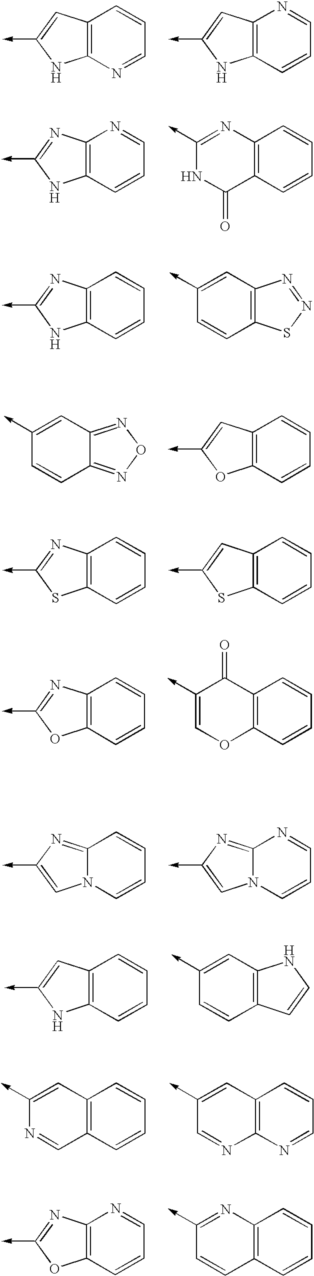

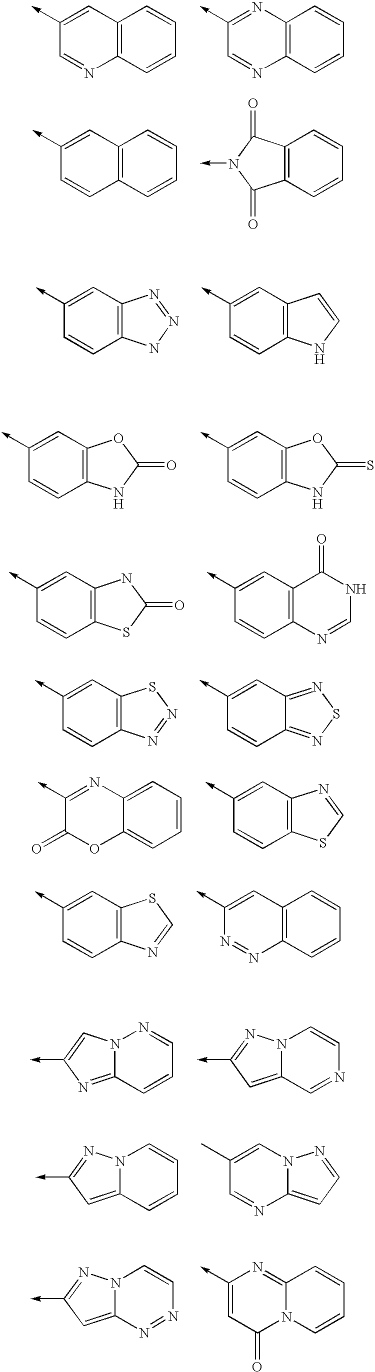

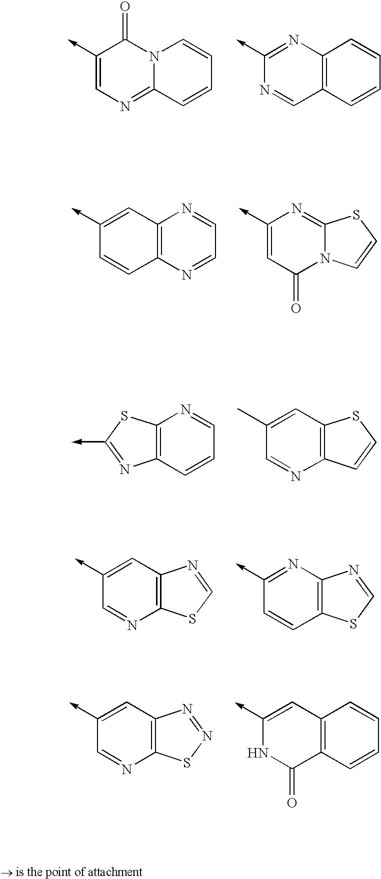

(a) is non aromatic
(2S)-2,3-dihydro-1H-indol-2-yl,
(2S)-2,3-dihydro-benzo[1,4]dioxine-2-yl,
3-(R,S)-3,4-dihydro-2H-benzo[1,4]thiazin-3-yl,
3-(R)-2,3-dihydro-[1,4]dioxino[2,3-b]pyridin-3-yl,
3-(S)-2,3-dihydro-[1,4]dioxino[2,3-b]pyridin-3-yl,
2,3-dihydro-benzo[1,4]dioxan-2-yl,
3-substituted-3H-quinazolin-4-one-2-yl,

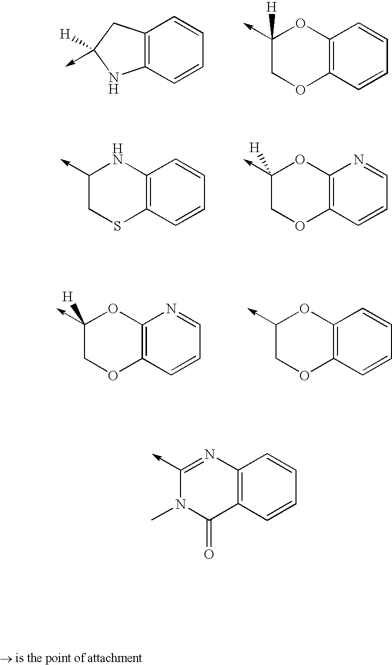

(b) is non aromatic
1,1,3-trioxo-1,2,3,4-tetrahydrol l⁶-benzo[1,4]thiazin-6-yl,
benzo[1,3]dioxol-5-yl,
2,3-dihydro-benzo[1,4]dioxin-6-yl
2-oxo-2,3-dihydro-benzooxazol-6-yl (benzoxazole-2(3H)-one-6-yl; 6-substituted benzoxazole-2(3H)-one)
3-substituted-3H-benzooxazol-2-one-6-yl,
3-substituted-3H-benzooxazole-2-thione-6-yl,
3-substituted-3H-benzothiazol-2-one-6-yl,
4H-benzo[1,4]oxazin-3-one-6-yl(3-oxo-3,4-dihydro-2H-benzo[1,4]oxazin-6-yl),
4H-benzo[1,4]thiazin-3-one-6-yl(3-oxo-3,4-dihydro-2H-benzo[1,4]thiazin-6-yl),
4H-benzo[1,4]oxazin-3-one-7-yl,
4-oxo-2,3,4,5-tetrahydro-benzo[1,5]thiazepine-7-yl,
5-oxo-2,3-dihydro-5H-thiazolo[3,2-a]pyrimidin-6-yl,
1H-pyrido[2,3-b][1,4]thiazin-2-one-7-yl(2-oxo-2,3-dihydro-1H-pyrido[2,3-b][1,4]thiazin-7-yl),
2,3-dihydro-1H-pyrido[2,3-b][1,4]thiazin-7-yl,
2-oxo-2,3-dihydro-1H-pyrido[3,4-b]thiazin-7-yl,
2,3-dihydro-[1,4]dioxino[2,3-b]pyridin-6-yl,
2,3-dihydro-[1,4]dioxino[2,3-c]pyridin-7-yl,
2,3-dihydro-[1,4]dioxino[2,3-b]pyridin-7-yl,
3,4-dihydro-2H-benzo[1,4]oxazin-6-yl,
3,4-dihydro-2H-benzo[1,4]thiazin-6-yl,
3-oxo-3,4-dihydro-2H-pyrido[3,2-b][1,4]oxazin-6-yl,
3,4-dihydro-2H-pyrido[3,2-b][1,4]thiazin-6-yl,
3-oxo-3,4-dihydro-2H-pyrido[3,2-b][1,4]thiazin-6-yl,
3,4-dihydro-1H-quinolin-2-one-7-yl,
3,4-dihydro-1H-quinoxalin-2-one-7-yl,
6,7-dihydro-4H-pyrazolo[1,5-a]pyrimidin-5-one-2-yl,
1,2,3,4-tetrahydro-[1,8]naphthyridin-7-yl,
2-oxo-3,4-dihydro-1H-[1,8]naphthyridin-6-yl,
6-oxo-6,7-dihydro-5H-8-thia-1,2,5-triaza-naphthalen-3-yl(3-substituted 5H-pyridazino[3,4-b][1,4]thiazin-6(7H)-one),
2-oxo-2,3-dihydro-1H-pyrido[3,4-b][1,4]oxazin-7-yl,
2-oxo-2,3-dihydro-1H-pyrido[2,3-b][1,4]oxazin-7-yl,
6,7-dihydro-[1,4]dioxino[2,3-d]pyrimidin-2-yl,
[1,3]oxathiolo[5,4-c]pyridin-6-yl,
3,4-dihydro-2H-pyrano[2,3-c]pyridine-6-yl,
2,3-dihydro[1,4]oxathiino[2,3-c]pyridine-7-yl,
6,7-dihydro[1,4]dioxino[2,3-c]pyridazin-3-yl,
2-substituted 1H-pyrimido[5,4-b][1,4]oxazin-7(6H)-one,
2-substituted 5,6-dihydropyrido[2,3-c]pyrimidin-7(1H)-one,
6,7-dihydro[1,4]oxathiino[2,3-c]pyridazin-3-yl,
6,7-dihydro-5H-pyrano[2,3-c]pyridazin-3-yl,
5,6-dihydrofuro[2,3-c]pyridazin-3-yl,
2,3-dihydrofuro[2,3-c]pyridin-5-yl,
7-substituted 2H-chromen-2-one,
7-substituted 2H-pyrano[2,3-b]pyridin-2-one,
2-substituted 6,7-dihydro-5H-pyrano[2,3-c]pyrimidine,
8-substituted 2H-pyrido[1,2-a]pyrimidin-2-one,
2,3-dihydro-1-benzofuran-5-yl,
2,3-dihydro-1H-pyrido[3,4-b][1,4]oxazin-7-yl

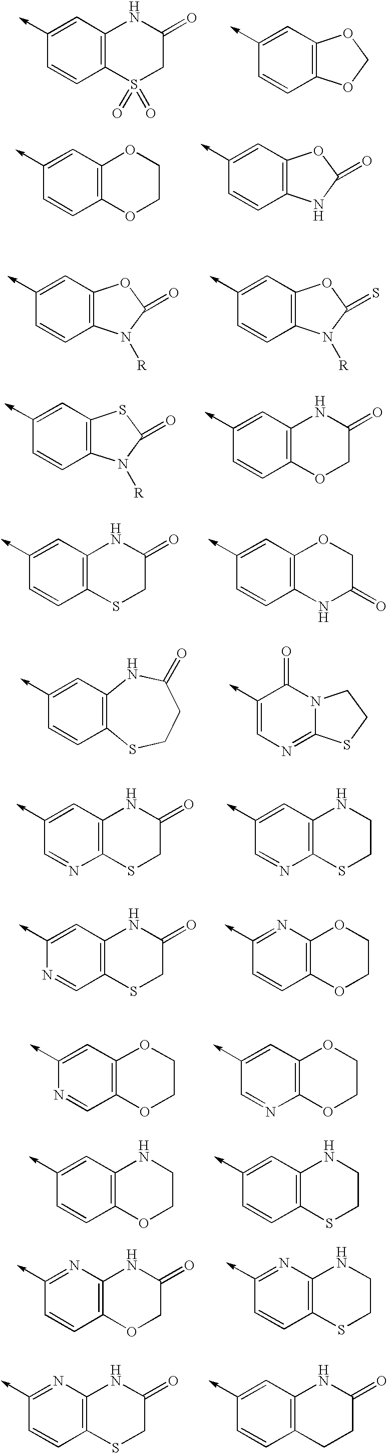

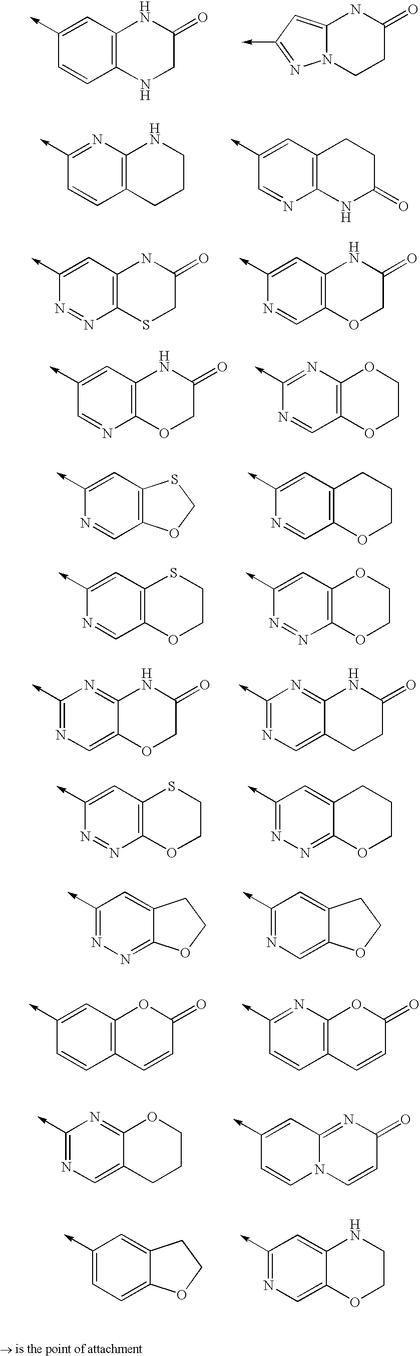

In some embodiments R¹³ is H if in ring (a) or in addition (C₁₋₄)alkyl such as methyl or isopropyl when in ring (b). More particularly, in ring (b) R¹³ is H when NR¹³ is bonded to X³ and (C₁₋₄)alkyl when NR¹³ is bonded to X⁵.

In further embodiments R¹⁴ and R¹⁵ are independently selected from hydrogen, halo, hydroxy, (C₁₋₄) alkyl, (C₁₋₄)alkoxy, nitro and cyano. More particularly R¹⁵ is hydrogen.

More particularly each R¹⁴ is selected from hydrogen, chloro, fluoro, hydroxy, methyl, methoxy, nitro and cyano. Still more particularly R¹⁴ is selected from hydrogen, fluorine or nitro.

Most particularly R¹⁴ and R¹⁵ are each H.

Particular groups R⁵ include:
[1,2,3]thiadiazolo[5,4-b]pyridin-6-yl
1H-pyrrolo[2,3-b]pyridin-2-yl
2,3-dihydro-[1,4]dioxino[2,3-b]pyridin-6-yl
2,3-dihydro-[1,4]dioxino[2,3-b]pyridin-7-yl
2,3-dihydro-[1,4]dioxino[2,3-c]pyridin-7-yl
2,3-dihydro-benzo[1,4]dioxin-6-yl
2-oxo-2,3-dihydro-1H-pyrido[2,3-b][1,4]oxazin-7-yl
2-oxo-2,3-dihydro-1H-pyrido[2,3-b][1,4]thiazin-7-yl
3,4-dihydro-2H-benzo[1,4]oxazin-6-yl
3-methyl-2-oxo-2,3-dihydro-benzooxazol-6-yl(6-substituted 3-methyl-1,3-benzoxazol-2(3H)-one)
3-oxo-3,4-dihydro-2H-benzo[1,4]oxazin-6-yl
3-oxo-3,4-dihydro-2H-pyrido[3,2-b][1,4]oxazin-6-yl(6-substituted 2H-pyrido[3,2-b][1,4]oxazin-3(4H)-one)
3-oxo-3,4-dihydro-2H-benzo[1,4]thiazin-6-yl(4H-benzo[1,4]thiazin-3-one-6-yl)
4-oxo-4H-pyrido[1,2-a]pyrimidin-2-yl
6-nitro-benzo[1,3]dioxol-5-yl
7-fluoro-3-oxo-3,4-dihydro-2H-benzo[1,4]oxazin-6-yl
8-hydroxy-1-oxo-1,2-dihydro-isoquinolin-3-yl
8-hydroxyquinolin-2-yl
benzo[1,2,3]thiadiazol-5-yl
benzo[1,2,5]thiadiazol-5-yl
benzothiazol-5-yl
thiazolo-[5,4-b]pyridin-6-yl
3-oxo-3,4-dihydro-2H-pyrido[3,2-b][1,4]thiazin-6-yl(6-substituted 2H-pyrido[3,2-b][1,4]thiazin-3(4H)-one)
7-chloro-3-oxo-3,4-dihydro-2H-pyrido[3,2-b][1,4]thiazin-6-yl
7-chloro-3-oxo-3,4-dihydro-2H-pyrido[3,2-b][1,4]oxazin-6-yl(6-substituted 7-chloro-2H-pyrido[3,2-b][1,4]oxazin-3(4H)-one)
7-fluoro-3-oxo-3,4-dihydro-2H-pyrido[3,2-b][1,4]thiazin-6-yl
2-oxo-2,3-dihydro-1H-pyrido[3,4-b][1,4]thiazin-7-yl
[1,3]oxathiolo[5,4-c]pyridin-6-yl
3,4-dihydro-2H-pyrano[2,3-c]pyridine-6-yl
2,3-dihydro-5-carbonitro-1,4-benzodioxin-7-yl(7-substituted 2,3-dihydro-1,4-benzodioxin-5-carbonitrile)
2,3-dihydro[1,4]oxathiino[2,3-c]pyridine-7-yl
2,3-dihydro-1-benzofuran-5-yl
6,7-dihydro[1,4]dioxino[2,3-c]pyridazin-3-yl
2-substituted 1H-pyrimido[5,4-b][1,4]oxazin-7(6H)-one
2-substituted 5,6-dihydropyrido[2,3-d]pyrimidin-7(1H)-one
6-fluoro-2,3-dihydro-1,4-benzodioxin-7-yl
6,7-dihydro[1,4]oxathiino[2,3-c]pyridazin-3-yl
6,7-dihydro-5H-pyrano[2,3-c]pyridazin-3-yl
5,6-dihydrofuro[2,3-c]pyridazin-3-yl
2,3-dihydrofuro[2,3-c]pyridin-5-yl,
2-substituted 4-chloro-1H-pyrimido[5,4-b][1,4]oxazin-7(6H)-one
2-substituted 4-chloro-5,6-dihydropyrido[2,3-d]pyrimidin-7(1H)-one
2-substituted 4-methyl-5,6-dihydropyrido[2,3-d]pyrimidin-7(1H)-one
2-substituted 4-methyloxy-5,6-dihydropyrido[2,3-d]pyrimidin-7(1H)-one
7-substituted 2H-chromen-2-one
7-substituted 2H-pyrano[2,3-b]pyridin-2-one
4-chloro-6,7-dihydro-5H-pyrano[2,3-d]pyrimidin-2-yl
8-substituted 2H-pyrido[1,2-a]pyrimidin-2-one
6,7-dihydro-5H-pyrano[2,3-d]pyrimidin-2-yl)
5-chloro-1-benzothiophen-2-yl
6-chloro-1-benzothiophen-2-yl
1-benzothiophen-5-yl
1-methyl-1H-1,2,3-benzotriazol-6-yl
imidazo[2,1-b][1,3]thiazol-6-yl
4-methyl-3,4-dihydro-2H-1,4-benzoxazin-7-yl
1-methyl-1H-indol-2-yl
2,3-dihydro-1H-pyrido[3,4-b][1,4]oxazin-7-yl

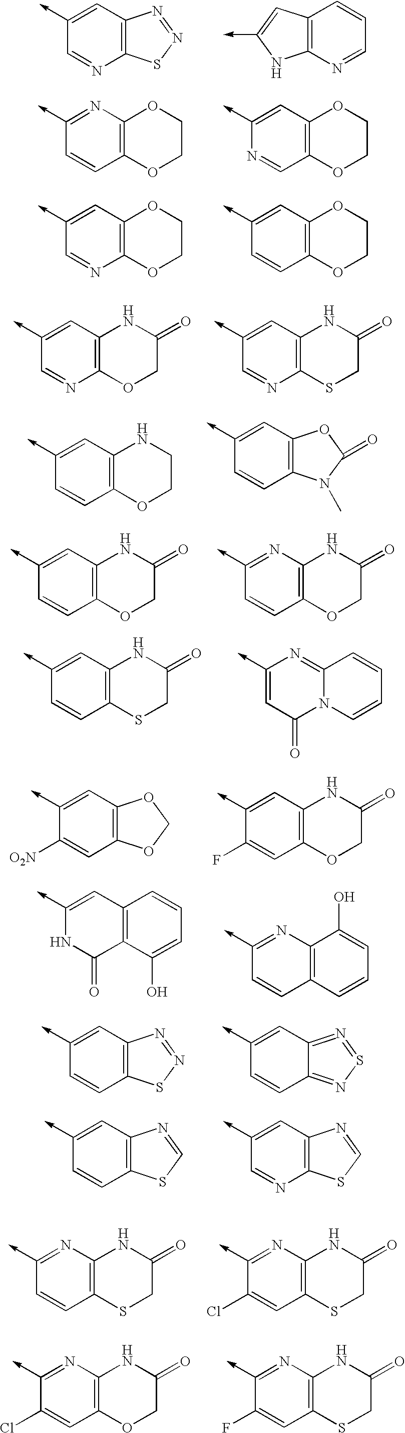

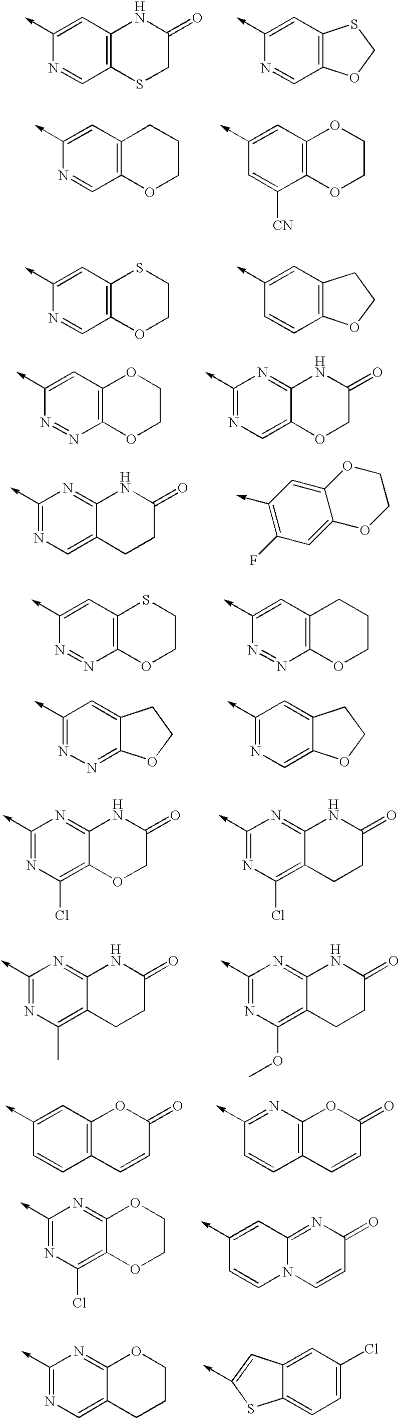

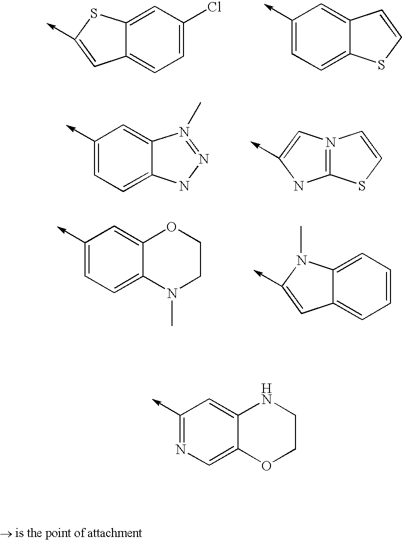

especially
6-substituted 2H-pyrido[3,2-b][1,4]oxazin-3(4H)-one
2,3-dihydro-[1,4]dioxino[2,3-c]pyridin-7-yl
[1,3]oxathiolo[5,4-c]pyridin-6-yl
3,4-dihydro-2H-pyrano[2,3-c]pyridine-6-yl
6-substituted 2H-pyrido[3,2-b][1,4]thiazin-3(4H)-one
6-substituted 7-chloro-2H-pyrido[3,2-b][1,4]oxazin-3(4H)-one
6,7-dihydro[1,4]dioxino[2,3-c]pyridazin-3-yl,

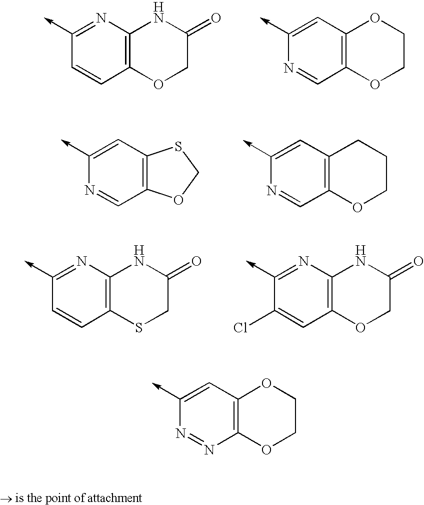

When used herein, the term “alkyl” includes groups having straight and branched chains, for instance, and as appropriate, methyl, ethyl, n-propyl, iso-propyl, n-butyl, iso-butyl, sec-butyl, t-butyl, pentyl and hexyl. The term ‘alkenyl’ should be interpreted accordingly.

Halo or halogen includes fluoro, chloro, bromo and iodo.

Haloalkyl moieties include 1-3 halogen atoms.

Compounds within the invention contain a heterocyclyl group and may occur in two or more tautomeric forms depending on the nature of the heterocyclyl group; all such tautomeric forms are included within the scope of the invention.

Some of the compounds of this invention may be crystallised or recrystallised from solvents such as aqueous and organic solvents. In such cases solvates may be formed. This invention includes within its scope stoichiometric solvates including hydrates as well as compounds containing variable amounts of water that may be produced by processes such as lyophilisation.

Furthermore, it will be understood that phrases such as “a compound of formula (I) or a pharmaceutically acceptable salt or N-oxide thereof” are intended to encompass the compound of formula (I), an N-oxide of formula (I), a pharmaceutically acceptable salt of the compound of formula (I) or any pharmaceutically acceptable combination of these.

Since the compounds of formula (I) are intended for use in pharmaceutical compositions it will readily be understood that in particular embodiments they are provided in substantially pure form, for example at least 60% pure, more suitably at least 75% pure and particularly at least 85%, especially at least 98% pure (% are on a weight for weight basis). Impure preparations of the compounds may be used for preparing the more pure forms used in the pharmaceutical compositions; these less pure preparations of the compounds should contain at least 1%, more suitably at least 5% and more particularly from 10% of a compound of the formula (I) or pharmaceutically acceptable salt or N-oxide thereof.

Particular compounds according to the invention include those mentioned in the examples and their pharmaceutically acceptable N-oxides and salts.

Pharmaceutically acceptable salts of the above-mentioned compounds of formula (I) include the acid addition or quaternary ammonium salts, for example their salts with mineral acids e.g. hydrochloric, hydrobromic, sulphuric nitric or phosphoric acids, or organic acids, e.g. acetic, fumaric, succinic, maleic, citric, benzoic, p-toluenesulphonic, methanesulphonic, naphthalenesulphonic acid or tartaric acids. Compounds of formula (I) may also be prepared as the N-oxide. The invention extends to all such derivatives.

Certain of the compounds of formula (I) may exist in the form of optical isomers, e.g. diastereoisomers and mixtures of isomers in all ratios, e.g. racemic mixtures. The invention includes all such forms, in particular the pure isomeric forms. The different isomeric forms may be separated or resolved one from the other by conventional methods, or any given isomer may be obtained by conventional synthetic methods or by stereospecific or asymmetric syntheses. Certain compounds of formula (I) may also exist in polymorphic forms and the invention includes such polymorphic forms.

In a further aspect of the invention there is provided a process for preparing compounds of formula (I), and pharmaceutically acceptable salts or N-oxides thereof, which process comprises reacting a compound of formula (II) with a compound of formula (IIIA):

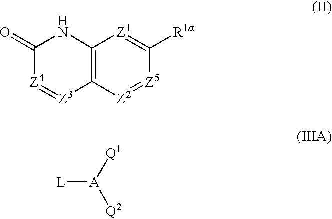

in which:
Z¹, Z², Z³, Z⁴, Z⁵, A and R^{1a} are as defined in formula (I), Q¹ and Q² are both attached to Y⁴ on A, Q¹ is H and Q² is N(R²⁰)R^{2′} or Q¹ and Q² together form ethylenedioxy or oxo, R²⁰ is UR⁵ or a group convertible thereto and R^{2′} is R² or a group convertible thereto, R², U and R⁵, are as defined in formula (I) and L is a leaving group such as bromo, to give a compound of formula (X):

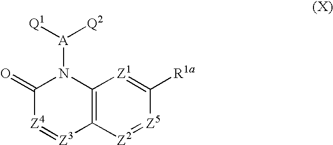

and thereafter optionally or as necessary converting Q¹ and Q² to NR²UR⁵, interconverting any variable groups, and/or forming a pharmaceutically acceptable salt, solvate or N-oxide thereof.

The reaction of (II) and (IIIA) is a palladium catalysed coupling using Pd₂(dba)₃ (tris(dibenzylideneacetone)dipalladium(0)) with xantphos (4,5-bis(diphenylphosphino)-9,9-dimethylxanthene) and Cs₂CO₃ (see Ligthart. G. et al, Journal of Organic Chemistry (2006), 71(1), 375-378).

Where Q¹ and Q² together form ethylenedioxy the ketal may be converted to the ketone (Q¹ and Q² together form oxo) by conventional acid hydrolysis treatment with eg aqueous HCl or trifluoroacetic acid and the conversion to NR²UR⁵ by conventional reductive alkylation with amine NHR^{2′}R²⁰ (see for example Nudelman, A., et al, Tetrahedron 60 (2004) 1731-1748) and subsequent conversion to the required substituted amine, or directly with NHR²UR⁵, such as with sodium triacetoxyborohydride in dichloromethane/methanol.

Conveniently one of R²⁰ and R^{2′} is an N-protecting group, such as such as t-butoxycarbonyl, benzyloxycarbonyl, 9-fluorenylmethyloxycarbonyl or trifluoroacetyl. This may be removed by several methods well known to those skilled in the art (for examples see “Protective Groups in Organic Synthesis, T. W. Greene and P. G. M. Wuts, Wiley-Interscience, 1999), for example conventional acid hydrolysis (e.g. trifluoroacetic acid/dichloromethane, hydrochloric acid/dichloromethane/methanol), or potassium carbonate/methanol. The free amine is converted to NR²UR⁵ by conventional means such as amide formation with an acyl derivative R⁵COW, for compounds where U is CO or, where U is CH₂, by alkylation with an alkyl halide R⁵CH₂-halide in the presence of base, acylation/reduction with an acyl derivative R⁵COW or reductive alkylation with an aldehyde R⁵CHO under conventional conditions (see for examples Smith, M. B.; March, J. M. Advanced Organic Chemistry, Wiley-Interscience 2001). Suitable conditions include sodium cyanoborohydride (in methanol/chloroform/acetic acid). If the amine (IIIA) is a hydrochloride salt then sodium acetate may be added to buffer the reaction. Sodium triacetoxyborohydride is an alternative reducing agent.

The appropriate reagents containing the required R⁵ group are known compounds or may be prepared analogously to known compounds, see for example WO02/08224, WO02/50061, WO02/56882, WO02/96907, WO2003087098, WO2003010138, WO2003064421, WO2003064431, WO2004002992, WO2004002490, WO2004014361, WO2004041210, WO2004096982, WO2002050036, WO2004058144, WO2004087145, WO2006014580, WO2004/035569, WO2004/089947, WO2003082835, WO2002026723, WO06002047, WO06010040, WO06017326, WO06012396, WO06017468, WO06020561, WO06132739, WO06134378, WO06137485, WO06081179, WO06081264, WO06081289, WO06081178, WO06081182, WO07016610, WO07081597, WO07071936, WO07115947, WO07118130, WO07122258, WO08006648, WO08003690, WO08009700, WO2007067511 and EP0559285.

The invention further provides compounds of formula (X) in which Q¹ is H and Q² is N(R²⁰)R^{2′} and R²⁰ is hydrogen.

Compounds of formula (II) may be prepared by the following Schemes 1a-c:

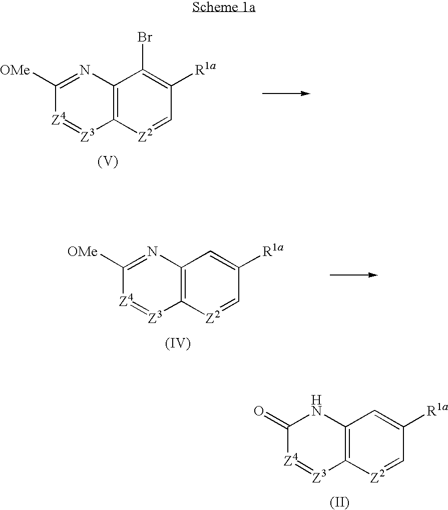

(where Z² is N and Z³ and Z⁴ are both CH). The bromo derivative (V) may be hydrogenated using Pd/C to give (IV). Demethylation with HBr affords the compound (II).

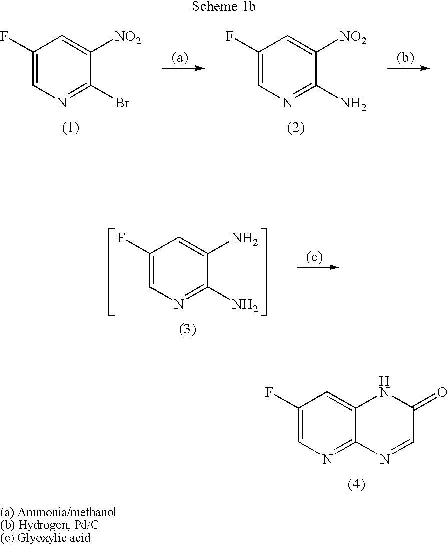

Reaction of commercially-available bromopyridine (1) with ammonia under pressure gives aminopyridine (2). Hydrogenation then affords unstable diaminopyridine (3) which is immediately condensed with glyoxylic acid to give 7-fluoropyrido[2,3-b]pyrazin-2(1H)-one (4).

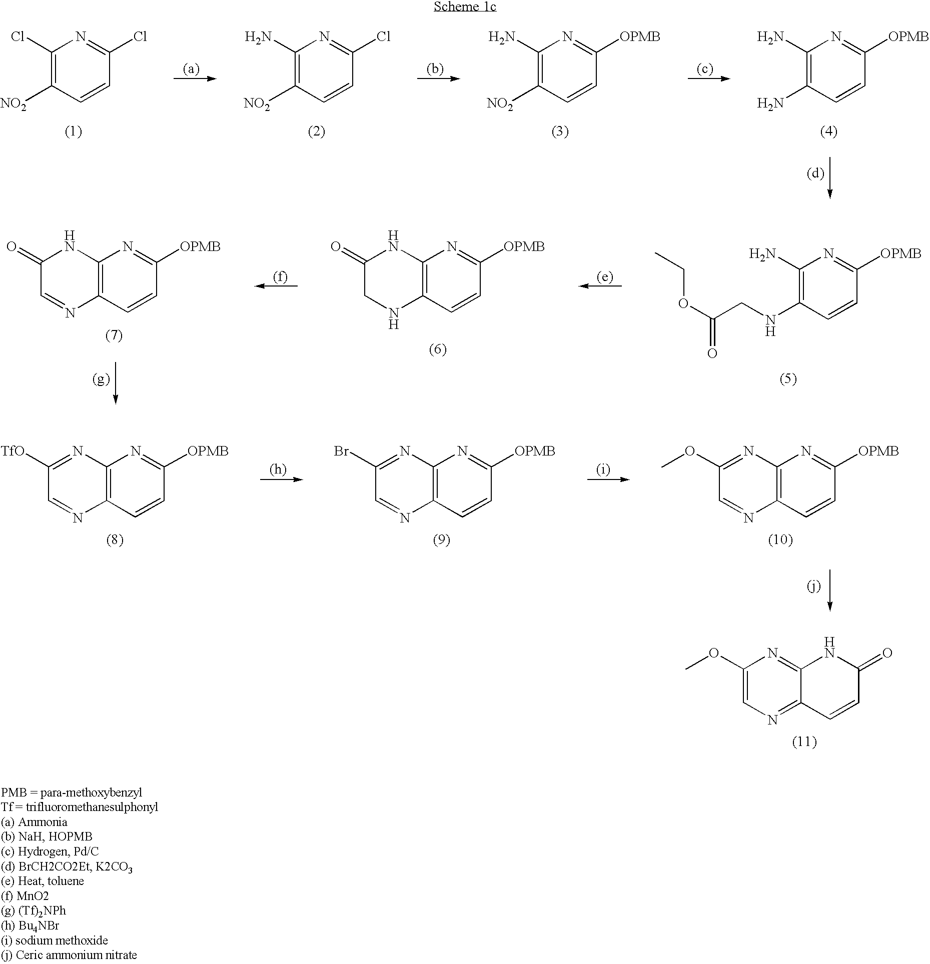

Chloropyridine (1) is reacted with ammonia to give amino pyridine (2) which is reacted with the anion of para-methoxybenzyl alcohol to give ether (3). Reduction of the nitro functionality gives diaminopyridine (4) which is then alkylated with bromoacetate to give ethyl ester (5). Thermal cyclisation affords (6) which is then oxidised to give (7). This is converted to triflate (8) which is displaced with bromide affording (9). Displacement with methoxide gives bis-ether (10) and then cleavage of the para-methoxybenzyl ether with ceric ammonium nitrate affords 3-(methyloxy)pyrido[2,3-b]pyrazin-6(4H)-one (11).

In an alternative aspect of the invention there is provided a process for preparing compounds of formula (I) where Z¹ and Z³ are both nitrogen, and pharmaceutically acceptable salts or N-oxides thereof, which process comprises reacting a compound of formula (VIa) or (VIb):

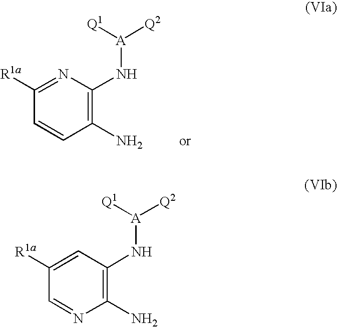

with (i) ethyl bromoacetate followed by cyclisation and oxidation or (ii) ethyl oxoacetate (ethylglyoxylate) followed by cyclisation, in which: A and R^{1a} are as defined in formula (I), Q¹ and Q² are both attached to Y⁴ on A, Q¹ is H and Q² is N(R²⁰)R^{2′} or Q¹ and Q² together form ethylenedioxy or oxo, R²⁰ is UR⁵ or a group convertible thereto and R^{2′} is R² or a group convertible thereto, and R², U and R⁵ are as defined in formula (I), and thereafter optionally or as necessary converting R²⁰ and R^{2′} to UR⁵ and R², interconverting any variable groups, and/or forming a pharmaceutically acceptable salt, solvate or N-oxide thereof.

The reaction variant (i) is a selective alkylation with ethyl bromoacetate under basic conditions (such as potassium carbonate) (see Yoshizawa, H. et al., Heterocycles (2004), 63(8), 1757-1763 for an example of this selectivity in the alkylation of 2,3-diaminopyridines), thermal cyclisation under strong basic conditions (such as potassium t-butoxide) and then oxidation with manganese dioxide under conventional conditions (see for examples Smith, M. B.; March, J. M. Advanced Organic Chemistry, Wiley-Interscience 2001).

The reaction variant (ii) may be carried out in toluene and the cyclisation effected by heating. Alternatively the imine can be reduced with sodium borohydride and then cyclised under strongly basic conditions (such as potassium t-butoxide) followed by oxidation as for variant (i).

Subsequent conversions may be carried out as described above.

Compounds of formula (VIa) and (VIb) may be prepared by the following Scheme 2:

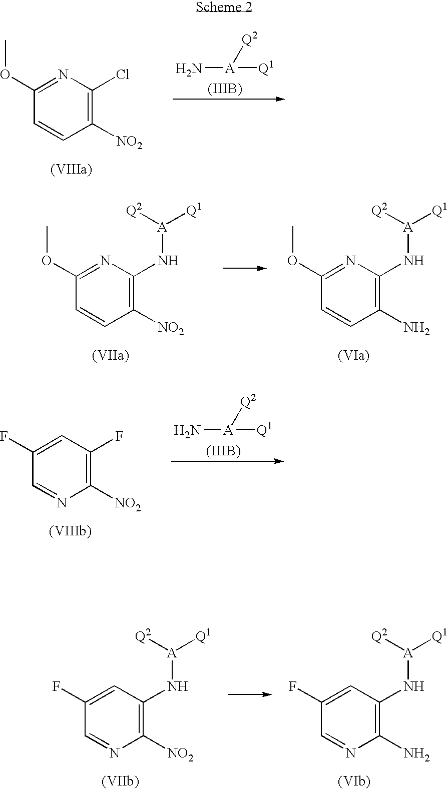

Conversion of a compound of formula (VIIIa/b) to (VIIa/b) takes place under conventional conditions optionally in the presence of a base such as sodium bicarbonate or in some cases a strong base such as NaH or potassium t-butoxide (see for examples Smith, M. B.; March, J. M. Advanced Organic Chemistry, Wiley-Interscience 2001). Compound (VIa/b) may then be prepared from (VIIa/b) via catalytic hydrogenation under conventional conditions (see for examples Smith, M. B.; March, J. M. Advanced Organic Chemistry, Wiley-Interscience 2001).

In an alternative aspect of the invention there is provided a process for preparing compounds of formula (I) where Z¹ and Z³ are both nitrogen, and pharmaceutically acceptable salts or N-oxides thereof, which process comprises reacting a compound of formula (IX) with a compound of formula (IIIA):

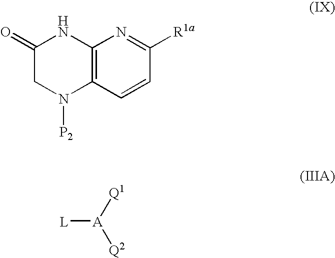

in which P₂ is a protecting group and the remaining variables are as previously defined, followed by oxidation of the resulting dihydro derivative. Conveniently L is chloro or iodo and the reaction is a palladium catalysed coupling using Pd₂(dba)₃ (tris(dibenzylideneacetone)dipalladium(0)) with xantphos (4,5-bis(diphenylphosphino)-9,9-dimethylxanthene) and Cs₂CO₃ (see Ligthart. G. et al, Journal of Organic Chemistry (2006), 71(1), 375-378). Alternatively L may be (HO)₂B and the coupling reaction catalysed by Cu(OAc)₂, see for example B. K. Singh et al, Organic Letters, 2006, 1863 (Chan coupling). Alternatively L may be bromo and the coupling effected with Cu(I) iodide, cyclohexyldiamine and K₂CO₃, in 1,4-dioxane at elevated temperature (eg 125° C.). Where (IIIA) carries an N-protecting group eg BOC then orthogonal protection requires P₂ to be a different protecting group such as CBZ. These protection groups can be clearly be reversed. The oxidation step is conveniently carried out with manganese dioxide under conventional conditions (see for examples Smith, M. B.; March, J. M. Advanced Organic Chemistry, Wiley-Interscience 2001).

Compounds of formula (IX) may be prepared by the following scheme:

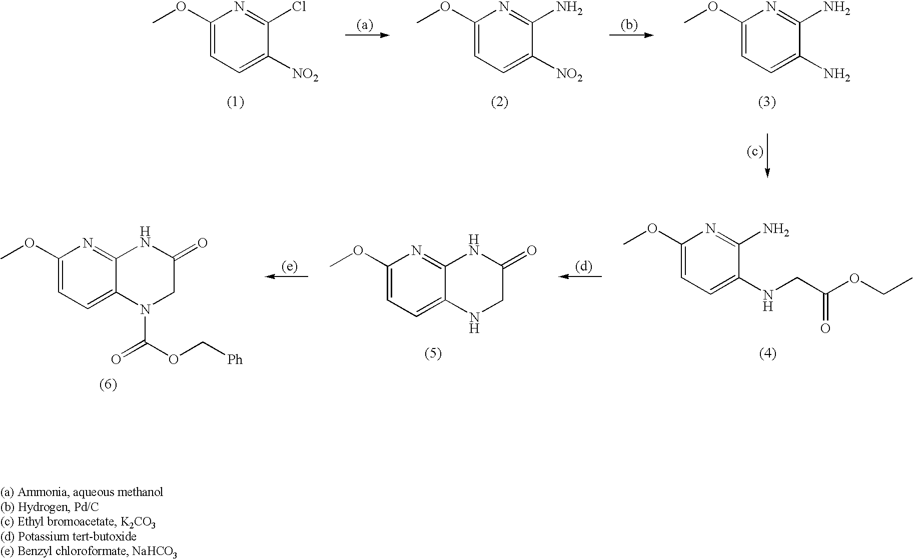

Amminolysis of chloropyridine (1) affords aminopyridine (2) which is hydrogenated to give the labile diaminopyridine (3). Alkylation with bromoacetate affords (4) which is cyclised using potassium tert-butoxide giving (5). Protection with benzyl chloroformate affords (6).

Compounds of formula (I) where Z¹ is N, Z² is CR^{1b}, Z³ is O and Z⁴ is CH₂ may be prepared by the following scheme

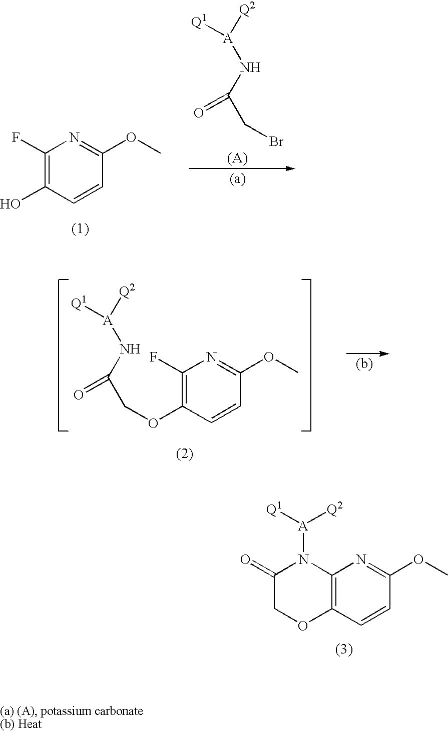

O-Alkylation of phenol (1) (see Reiffenrath, V. et al, Angewandte Chemie (1994), 106(13), 1435-8 for the preparation of fluoropyridines with this type of substitution pattern) with bromacetamido intermediate (A) affords ether (2) which cyclises under the basic conditions of the reaction to give key oxazinone intermediate (3) (see Ma, T. et al, Hecheng Huaxue, 2003, 11(6), 513 for examples of pyrido[1,4]oxazinones prepared by this methodology) and thereafter optionally or as necessary converting Q¹ and Q² to NR²UR⁵, interconverting any variable groups, and/or forming a pharmaceutically acceptable salt, solvate or N-oxide thereof.

Interconversions of R^{1d}, R^{1b}, R², A and R⁵ are conventional. For example R^{1a} alkoxycarbonyl may be converted to R^{1a} carboxy by hydrolysis, which in turn may be converted to R^{1a} aminocarbonyl and cyano by conventional procedures. R^{1a} halo may be introduced by conventional halogenation reactions eg chlorination with chlorosuccinimide in acetic acid to introduce a chloro group at R^{1b}. In compounds which contain an optionally protected hydroxy group, suitable conventional hydroxy protecting groups which may be removed without disrupting the remainder of the molecule include acyl and alkylsilyl groups. N-protecting groups are removed by conventional methods.

For example R^{1a} or R^{1b} methoxy is convertible to R^{1a} or R^{1b} hydroxy by treatment with lithium and diphenylphosphine (general method described in Ireland et al, J. Amer. Chem. Soc., 1973, 7829) or HBr. Alkylation of the hydroxy group with a suitable alkyl derivative bearing a leaving group such as halide, yields R^{1a} or R^{1b} substituted alkoxy. R^{1a} halogen is convertible to other R^{1a} by conventional means, for example to hydroxy, alkylthiol (via thiol) and amino using metal catalysed coupling reactions, for example using copper as reviewed in Synlett (2003), 15, 2428-2439 and Angewandte Chemie, International Edition, 2003, 42(44), 5400-5449. R^{1a} fluoro may be converted to methoxy by treatment with sodium methoxide in methanol. R^{1b} halo such as bromo may be introduced by the general method of M. A. Alonso et al, Tetrahedron 2003, 59(16), 2821 or P. Imming et al, Eur. J. Med. Chem., 2001, 36 (4), 375. R^{1b} halo such as chloro may be introduced by treatment with N-chlorosuccinimide. R^{1a} or R^{1b} halo such as bromo may be converted to cyano by treatment with copper (I) cyanide in N,N-dimethylformamide. R^{1a} or R^{1b} carboxy may be obtained by conventional hydrolysis of R^{1a} or R^{1b} cyano, and the carboxy converted to hydroxymethyl by conventional reduction.

Compounds of formulae (II), (V) and (IX) are known compounds or may be prepared analogously to known compounds, for example quinazolinone and quinazolines may be prepared by standard routes as described by T. A. Williamson in Heterocyclic Compounds, 6, 324 (1957) Ed. R. C. Elderfield. Napthyridines may be prepared by routes analogous to those described in Comprehensive Heterocyclic Chemistry, Volume 2, Ed A. J. Boulton and A. McKillop.

4-Halogeno derivatives such as (V) are commercially available, or may be prepared by methods known to those skilled in the art. A-4-bromo-substituent may be prepared from the quinolin- or naphthyridin-4-one by reaction with phosphorus tribromide (PBr₃) in DMF. A 4-chloroquinazoline is prepared from the corresponding quinazolin-4-one by reaction with phosphorus oxychloride (POCl₃) or phosphorus pentachloride, PCl₅.

For compounds of formulae (II) and (V) see for example WO2004/035569, WO2004/089947, WO02/08224, WO02/50061, WO02/56882, WO02/96907, WO2003087098, WO2003010138, WO2003064421, WO2003064431, WO2004002992, WO2004002490, WO2004014361, WO2004041210, WO2004096982, WO2002050036, WO2004058144, WO2004087145, WO2003082835, WO2002026723, WO06002047, WO06014580, WO06134378, WO06137485.

Compounds of formulae (IIIA) and (IIIB), including compounds in which Q¹ is H and Q² is N(R²⁰)R^{2′} and R²⁰ is hydrogen or Q¹ and Q² together form oxo are known compounds or may be prepared analogously to known compounds.

Where A is a group:

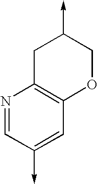

compounds of formula (I) may be prepared by reaction of a compound of formula (II) with a compound of formula (IIIA′):

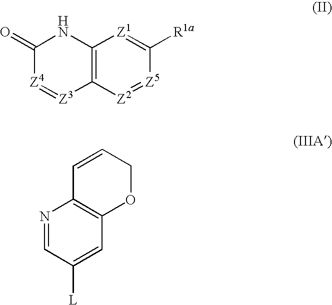

The compound of formula (IIIA′) where L is B(OH)₂ may be prepared by the following scheme:

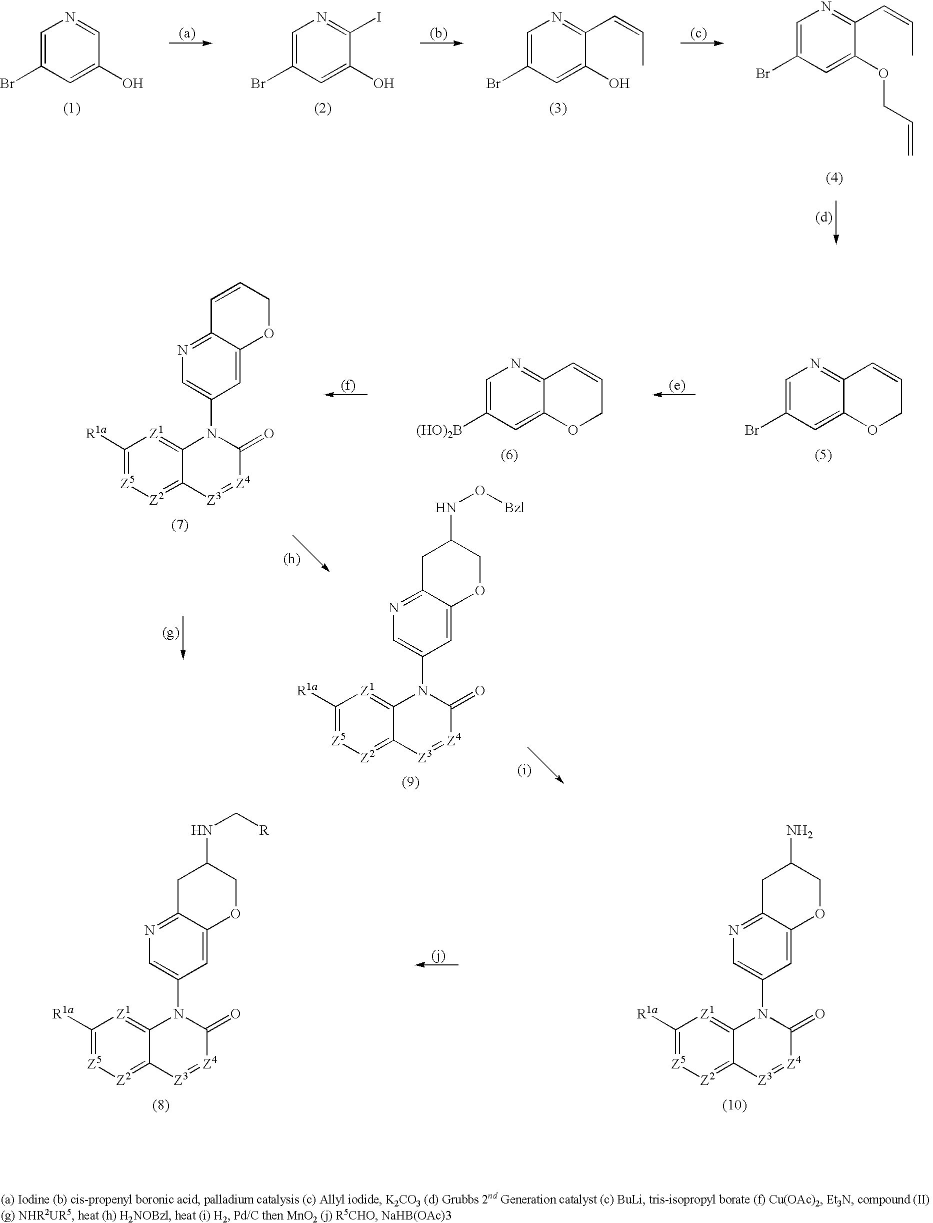

Hydroxy pyridine (1) can be iodinated to give iodopyridine (2), which is reacted with cis-propenyl boronic acid under palladium catalysis to give (3). Allylation to give (4) sets up the substrate for the metathesis cyclisation using Grubbs 2^{nd} Generation catalyst affording pyranopyridine (5). This can be metallated and the lithio species transformed to the boronic acid (6). Chan coupling with the compound of formula (II) then generates the N-aryl intermediate (7). Compounds of formula (I) may be accessed either directly, by a pseudo Michael addition to the olefin using the amine NHR²UR⁵, or indirectly, via pseudo Michael addition of O-benzylhydroxylamine followed by reduction and rearomatisation to amine (10) then conversion to NHR²UR⁵ as described above such as standard reductive alkylation.

Compounds of formula (IIIB) in which A is a group (ii) where A₁, A₂ and A₃ are each CR³ and Y₃, Y₅ and Y₆ are each CHR³ and Y₄ is C—OH may be prepared by the following Scheme:

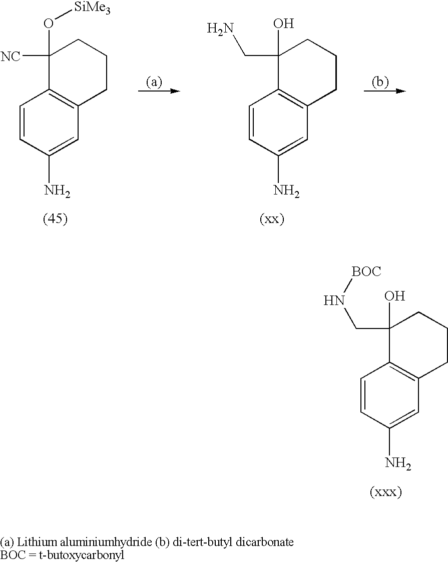

Reduction of cyano derivative (45) followed by hydrolytic cleavage of the silyl protecting group affords aminoalcohol (xx) which can be selectively protected on the aliphatic amine to give (xxx).

Compounds of formulae (IIIA) or (IIIB) where A₁, A₂ or A₃ are heteroatoms as defined in formula (I) may be prepared by conventional routes, such as described in Schemes 3-8 below:

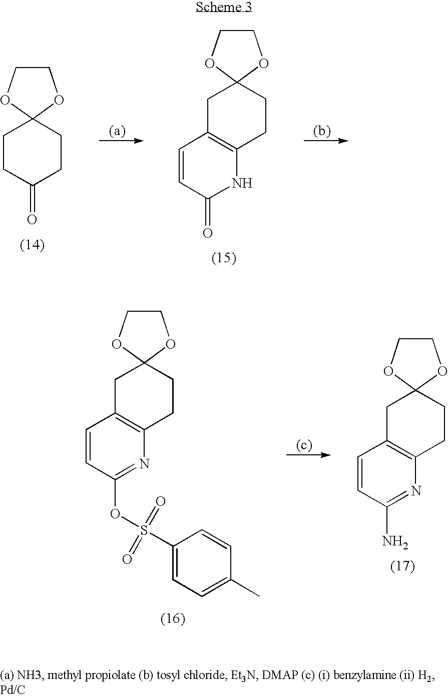

Condensation of ketone (14) with ammonia and methyl propiolate gives pyridine (15) which can be converted into the corresponding tosylate (16) (known in the literature, see Glase, S. et al, Journal of Medicinal Chemistry (1995), 38(16), 3132-7) and then to aminopyridine (17) by displacement with benzylamine and hydrogenolysis (again see Glase, S. et al, (1995) for related chemistry). Alternatively, chlorination of pyridine (15) followed by aminolysis (see Li, J. et al., Journal of the American Chemical Society (2005), 127(36), 12657-12665) may also afford amine (17).

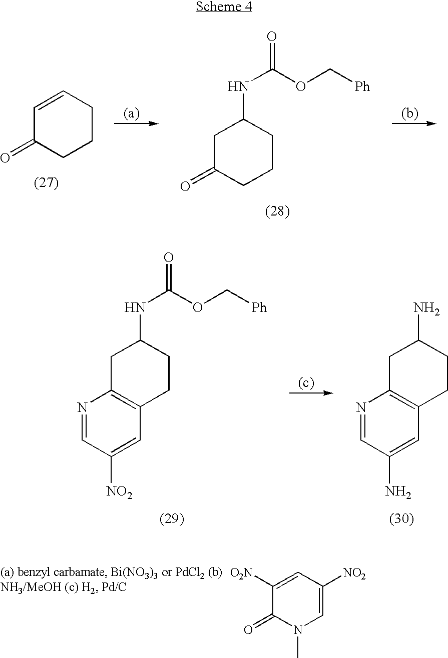

Cyclohexanone (27) can be converted to carbamate (28) and hence to nitropyridine (29) following a literature procedure (Drescher, K. et al, WO2006040178). Hydrogenation then reduces the nitro group to amino with concomitant deprotection of the second amino group to give (30).

R³ groups may be introduced into the ring in (30) (Scheme 4) by the following Scheme 4a:

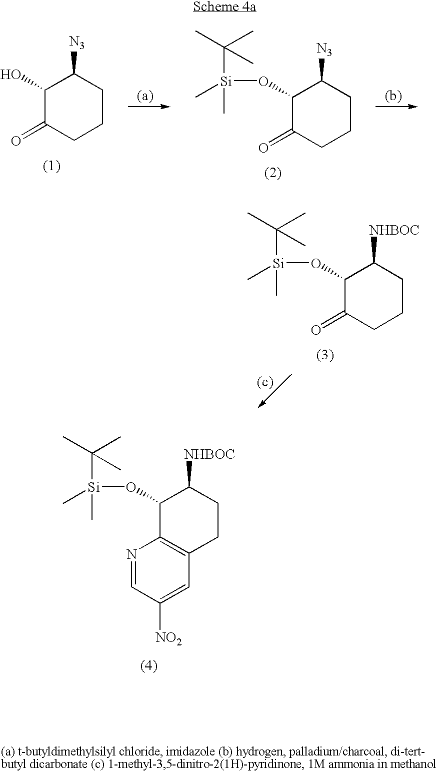

Silylation of racemic trans hydroxyketone (1) (prepared by the methodology of Murahashi, S. et al, Journal of Organic Chemistry (1993), 58(11), 2929-30) gives ether (2). This is hydrogenated in the presence of di-tert-butyl dicarbonate to give the protected aminoketone (3). This is then subjected to treatment with 1-methyl-3,5-dinitro-2(1H)-pyridinone in 1M ammonia in methanol to afford the nitropyridine (4) which can be reduced to the amine as in Scheme 4.

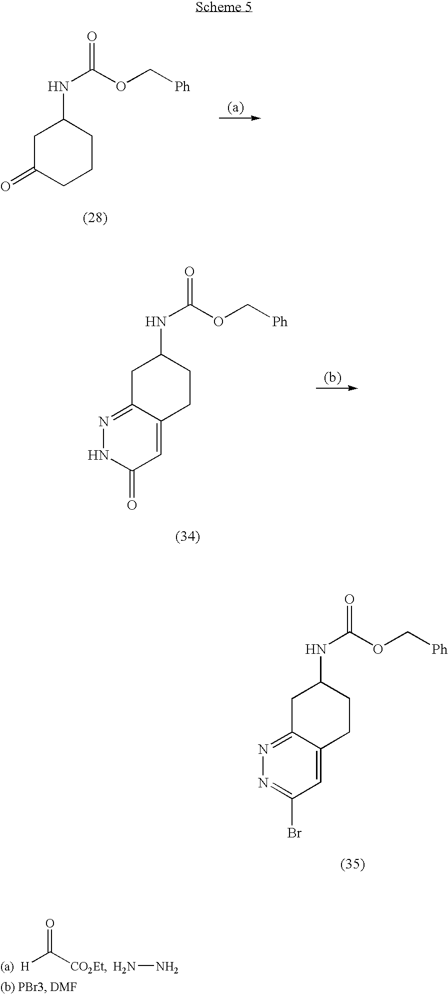

Ketone (28) may be condensed with hydrazine and glyoxylic acid according to the method of Costantino, L. et al., Farmaco (2000), 55(8), 544-552 to give (34), which can be converted to the corresponding bromide by treatment with phosphorus tribromide (35) and then transformed into representative final compounds (IIIA) or (IIIB) as described above.

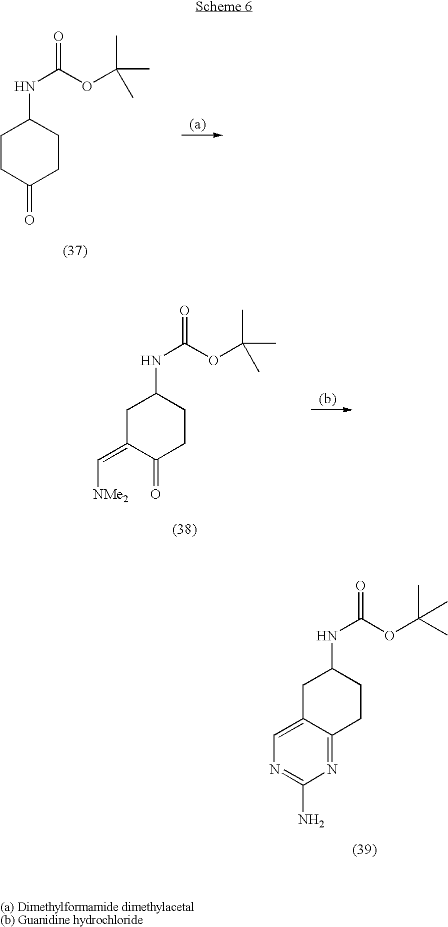

Ketone (37) is condensed with dimethylformamide dimethylacetal or tris(dimethylamino)methane to give (38) and then guanidine hydrochloride to give (39) (see Marinko, P. et al., Journal of Heterocyclic Chemistry (2000), 37(2), 405-409 for a closely related example of this type of heterocycle formation). Transformation into representative final compounds (IIIA) or (IIIB) is as described above.

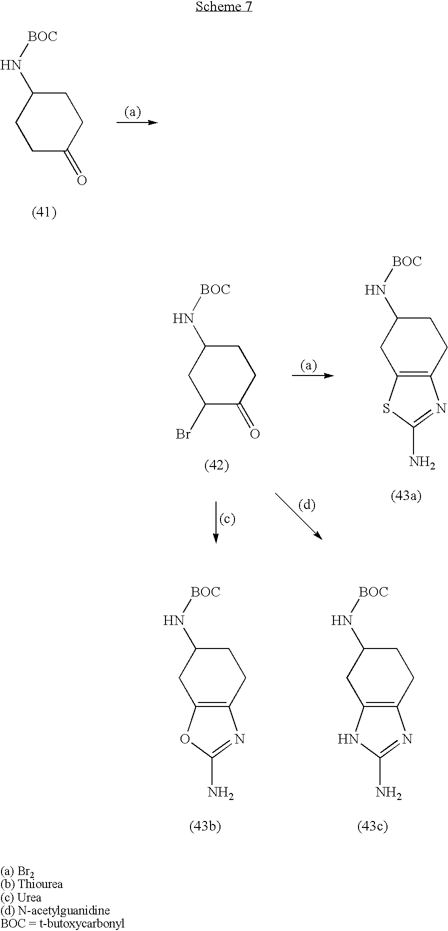

Ketone (41) can be brominated to give (42). Condensation with thiourea affords key aminothiazole (43a). For examples of this type of chemistry see Kanwar, S. et al, WO2006117614, Rao, D. (2004) GB 2394951 and finally Schneider, C. et al, Journal of Medicinal Chemistry (1987), 30(3), 494-8. Reaction of (42) with urea similarly gives the corresponding aminooxazole (43b) (see for example, Xiang, J. et al, Bioorganic & Medicinal Chemistry Letters (2005), 15(11), 2865-2869. Similarly reaction with N-acetylguanidine affords the corresponding aminoimidazole derivative (43c), for example see Ahmad, S. et al., Bioorganic & Medicinal Chemistry Letters (2004), 14(1), 177-180.

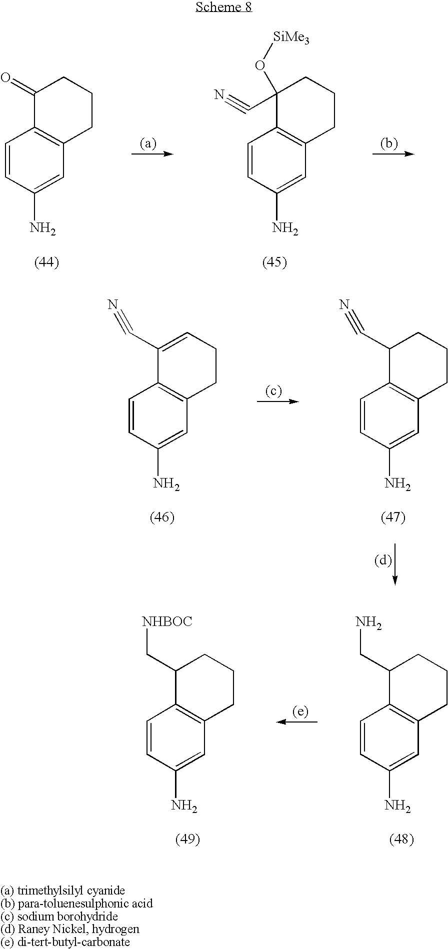

Reaction of commercially available ketone (44) with trimethylsilyl cyanide affords cyanohydrin (45) (see Coe, J. et al, WO 2005007630 for a closely related example) which can be easily dehydrated with acid to give olefin (46) (see Napier, J. et al Application: EP 86-106200 19860506). Reduction with sodium cyanoborohydride according to the method of ltenbach, R. Journal of Medicinal Chemistry (2004), 47(12), 3220-3235 gives saturated analogue (47) then further reduction with a Raney nickel hydrogenation produces the diamino derivative (48) (see DeBernardis, J. et al, Journal of Medicinal Chemistry (1985), 28(10), 1398-404 for precedent). Preferential protection of the aliphatic amino group as a carbamate then affords (49).

R³ groups at Y₅ may be introduced by the following Scheme 9:

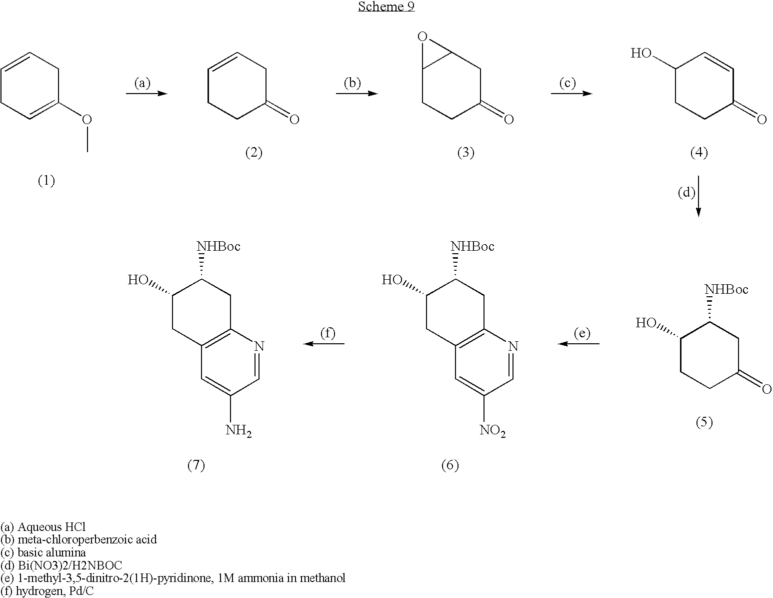

Hydrolysis of enol ether (1) affords unsaturated ketone (2) which can be epoxidised to give (3). Rearrangement using basic alumina gives hydroxyl enone (4) then Michael addition of 1,1-dimethylethyl carbamate affords (5). Cycloaddition using 1-methyl-3,5-dinitro-2(1H)-pyridinone produces nitropyridine (6) then hydrogenation gives aminopyridine (7).

Compounds of formula (IIIA) or (IIIB) where Y₃, Y₄ or Y₆ are X or CO as defined in formula (I) may be prepared by conventional routes, such as described in Schemes 10-18 below:

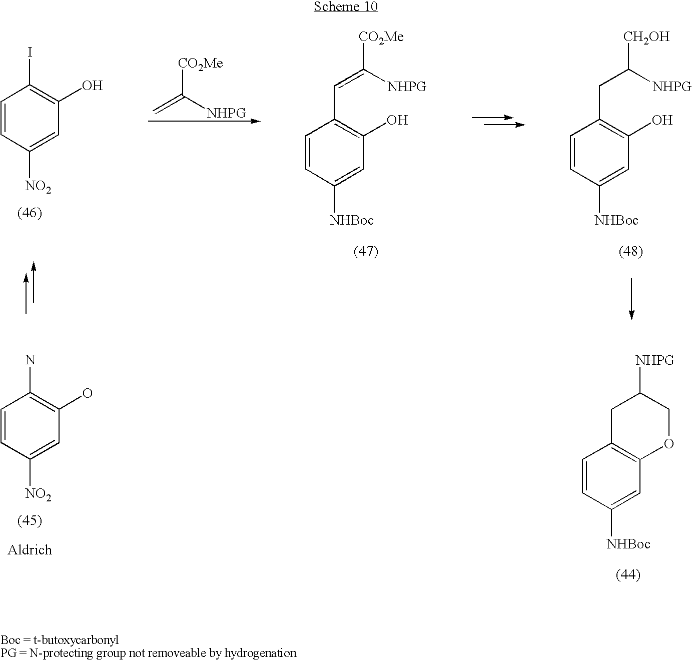

Commercially available amine (45) can be reacted with NaNO₂ and H₂SO₄ followed by KI to give (46) [see Tetrahedron 2002, 43(51), 9377-9380]. This iodide then may undergo a Heck reaction with a protected aminoacrylate to give compound (47) [see Org. lett. 2001, 3(13), 2053-2056]. The double bond of this acrylate may be reduced using palladium and hydrogen to give the saturated analogue [see Org. lett. 2001, 3(13), 2053-2056] followed by reduction of ester to give (48) by the use of either lithium triethylborohydride [see Org. lett. 2001, 3(13), 2053-2056] or NaBH₄+LiI [see Tetrahedron 2005, 61(45), 10748-10756]. Finally cyclisation, under Mitsunubo conditions may lead to the intermediate (44) [see Tetrahedron Asymmetry 2001, 12(12), 1689-1694].

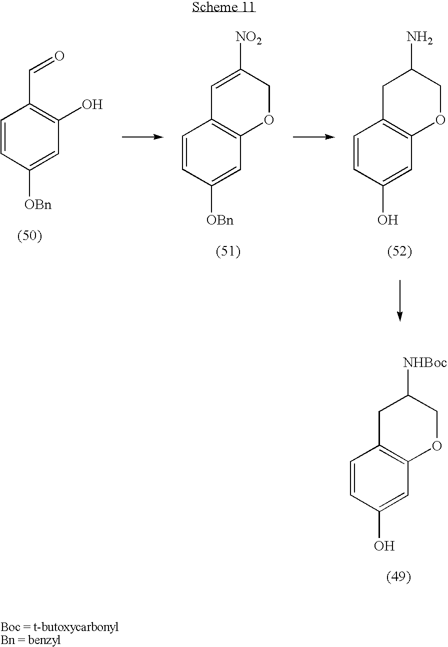

Phenol (49) may be synthesised by a number of routes. For instance, commercially available aldehyde (50) may condense with nitroethanol to give pyran (51) [J. Med. Chem. 2006, 49(23), 6848-6857]. Reduction of (51) using LiAlH₄ followed by reduction with palladium and hydrogen may give compound (52) [see Bioorg. & Med. Chem. Lett. 2004, 14(1), 47-50]. Protection of the primary amine could be possible using Boc₂O to give the desired phenol (49) [J. Med. Chem. 2006, 49(15), 4497-4511].

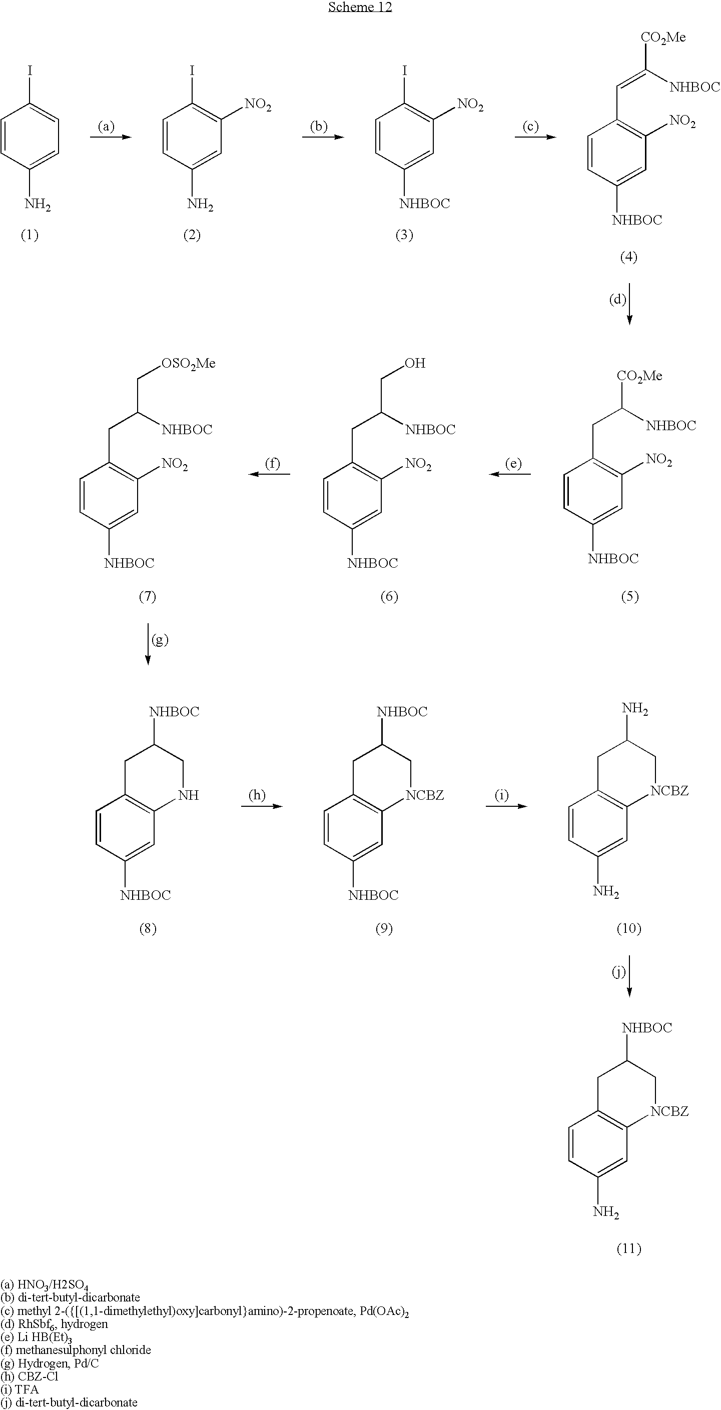

This route is based on the synthesis of related structures as described by RajanBabu, T. et al, Organic Letters, 2001, 3, 2053. Nitration of commercially-available aniline (1) gives meta-nitro derivative (2) which can be protected as (3). A Heck reaction with methyl 2-({[(1,1-dimethylethyl)oxy]carbonyl}amino)-2-propenoate catalysed by palladium (II) acetate gives (4) followed by selective reduction of the double bond using hydrogen and a Rhodium catalyst gives (5). The ester can then be reduced to primary alcohol (6) which can then be activated as mesylate (7). Hydrogenation of the nitro group with concomitant cyclisation gives (8). It is now necessary to protect the free amine with a CBZ group (9) before removing both the BOC protecting groups to give diamine (10). Selective protection of the aliphatic amino group then provides (11).

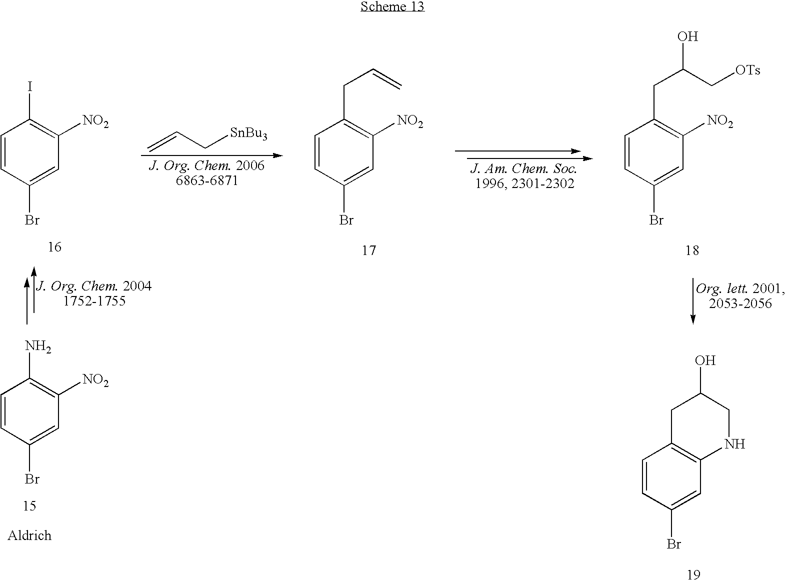

Commercially available amine (15) can be converted to the iodide (16) via diazotization followed by iodination [J. Org. Chem. 2004, 69(5), 1752-1755]. This resulting iodide may undergo a Stille coupling with allyl tin to give compound (17) [J. Org. Chem. 2006, 71(18), 6863-6871]. Dihydroxylation followed by selective protection with TsCl will give tosylate (14) [J. Am. Chem. Soc. 1996, 118(9), 2301-2302]. This nitro compound (18) can be reduced to the corresponding aniline with spontaneous cyclisation to give tetrahydroquinoline (19) [Org. lett. 2001, 3(13), 2053-2056]. Intermediate (19) may be protected on the nitrogen using benzyl chloroformate to give a CBZ intermediate. Activation of the alcohol as a mesylate and displacement with sodium azide affords the azide. Reduction of the azide and protection of the corresponding amine with di-tert-butyl dicarbonate affords the BOC protected primary amine of formula (IIIB).

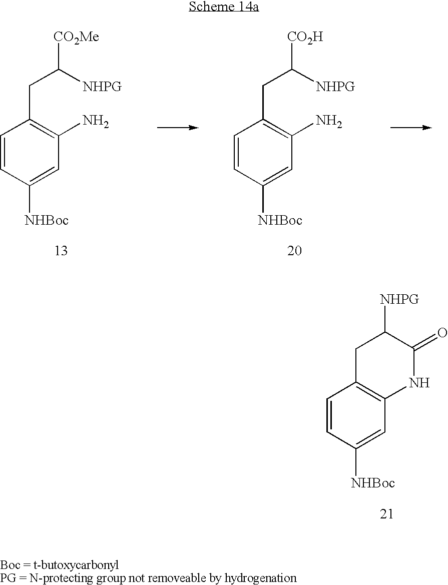

Compound (13) (Scheme 10) may be hydrolysed to the acid (20) [J. Org. Chem. 2006, 71(15), 5625-5630] and then cyclised via an amide coupling reaction [J. Heterocyclic Chem. 1982, 19(2), 401-406 or Chemical & Pharmaceutical Bulletin 2005, 53(11), 1387-1391] to give compound (21)

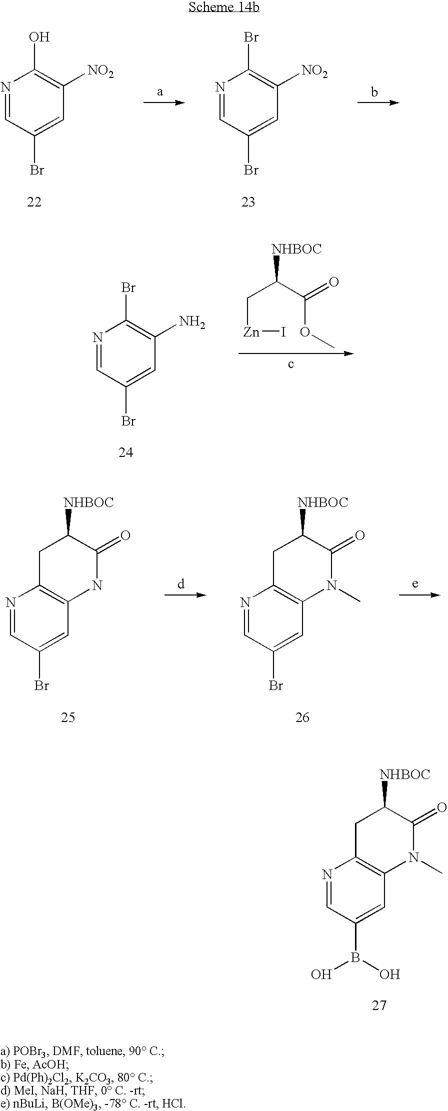

Compounds 26 and 27 of formula (IIIA) may be prepared by Scheme 14a above and then coupled with a compound of formula (II) or (IX) as described above. In particular, the coupling of compound 26 with a compound of formula (IX) may be carried out with Cu(I) iodide, cyclohexyldiamine and K₂CO₃, in 1,4-dioxane at elevated temperature (eg 125° C.). The coupling of compound 27 with a compound of formula (IX) may be effected with Cu(II) acetate and triethylamine in DCM at room temperature.

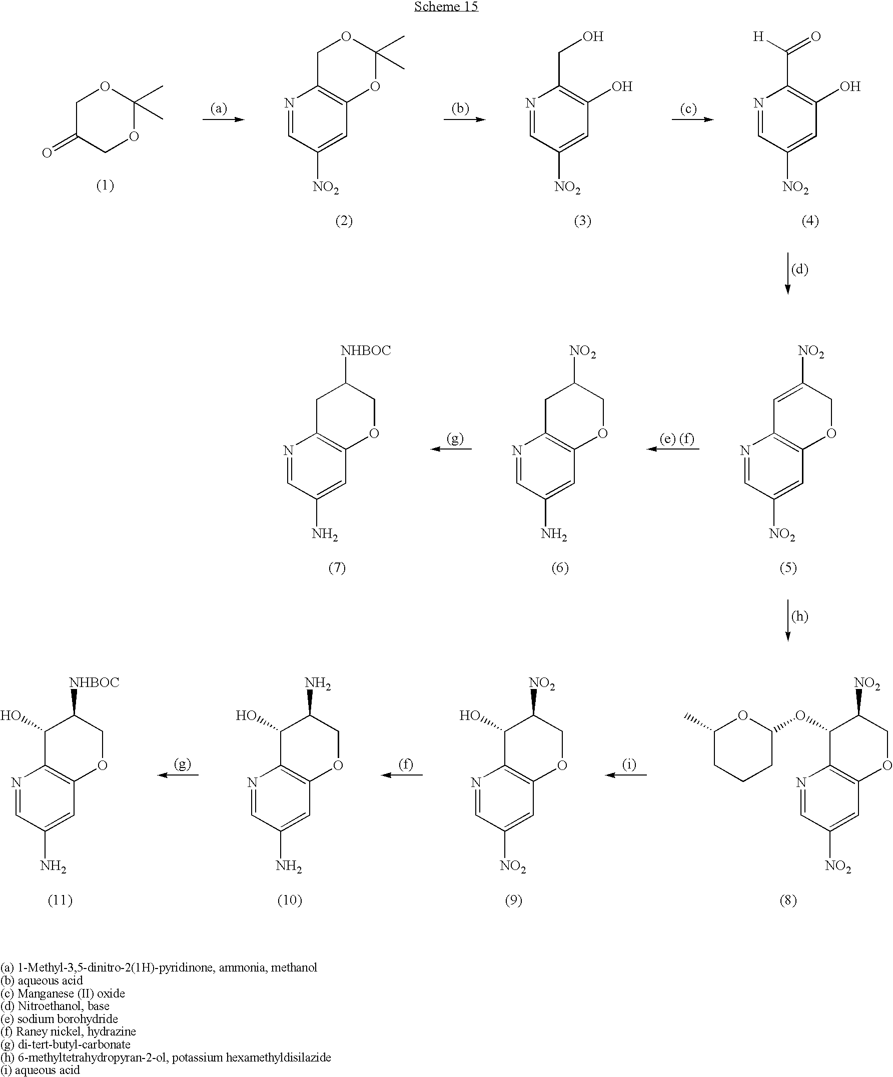

Reaction of 2,2-dimethyl-1,3-dioxan-5-one (1) with 1-methyl-3,5-dinitro-2(1H)-pyridinone (for precedent for formation of such nitropyridines from ketones see Harling J. et al, Syn. Comm., 2001, 31(5), 787) affords nitropyridine (2). Deprotection of the acetal affords diol (3) then selective oxidation of the benzylic alcohol gives aldehyde (4) (see Wang, P. et al, J. Med. Chem., 1990, 33, 608, for an example of this reaction on the des-nitro analogue). Condensation of (4) with nitroethanol then gives bicyclic ether (5) (see Cueva, J., Journal of Medicinal Chemistry (2006), 49(23), 6848-6857.) The double bond can be reduced with sodium borohydride and then both aromatic and aliphatic nitro functionalities can be reduced to amino using hydrazine and Raney nickel to give (6) (see Hatzenbuhler, N. et al., Journal of Medicinal Chemistry (2006), 49(15), 4785-4789). Selective protection of the aliphatic nitrogen is achievable with di-tert-butyl-carbonate to give (7). Alternatively (5) can be reacted with the anion of 6-methyltetrahydropyran-2-ol to produce acetal (8) (see Buchanon, D. J. et al, SynLett, 2005, (12), 1948 and Adderly, N. J. et al, Angew. Chem. Int. Ed. Eng., 2003, 42, 4241 for examples of this type of chemistry). The acetal may be removed with acid at this stage to give (9) or more preferably may be left until later on in the synthesis. Reduction of (9) with Raney nickel and hydrogen can give (10) which may be selectively protected to afford (11).

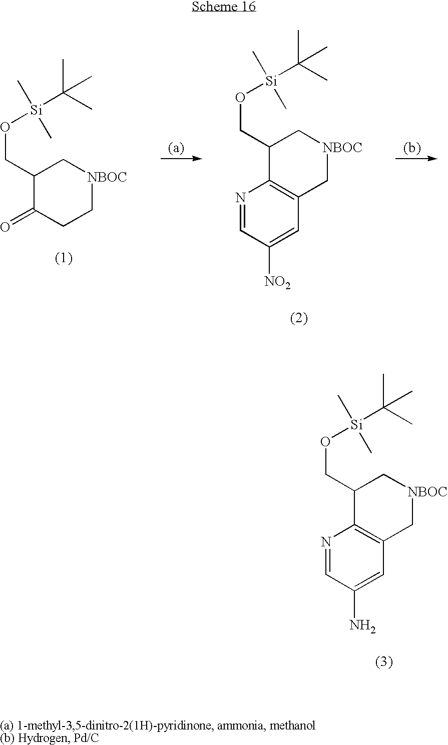

Treatment of (1) (see Ting, P. et al US 2005182095) with 1-methyl-3,5-dinitro-2(1H)-pyridinone in methanolic ammonia gives nitropyridine (2) which can be hydrogenated to aminopyridine (3).

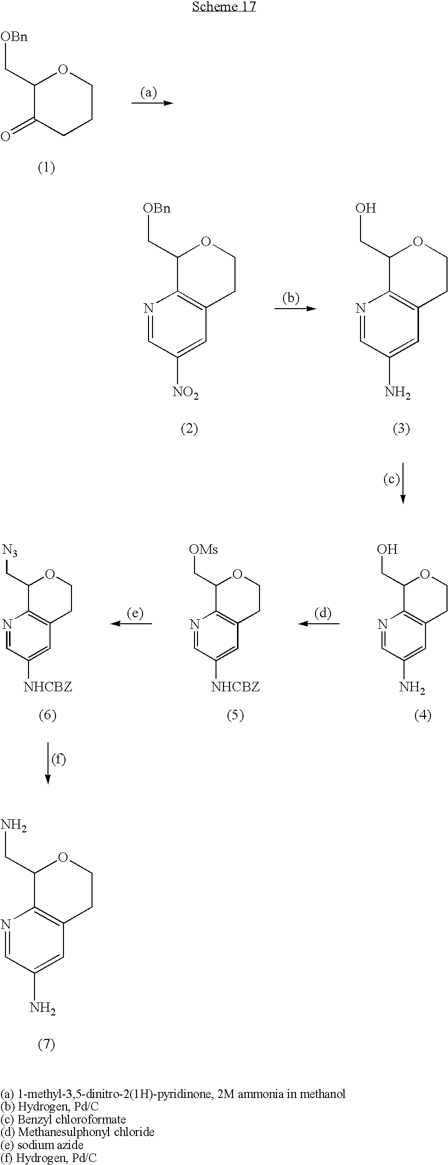

Reaction of ketone (1) (see Oishi, T. et al Synlett (1997), (8), 980-982 for the preparation of this intermediate) with 1-methyl-3,5-dinitro-2(1H)-pyridinone in 2M ammonia in methanol affords nitropyridine (2) which on hydrogenation affords alcohol (3). Protection of the amine affords (4) then mesylation gives mesylate (5). Displacement with sodium azide affords (6) then a second hydrogenation yields diamine (7).

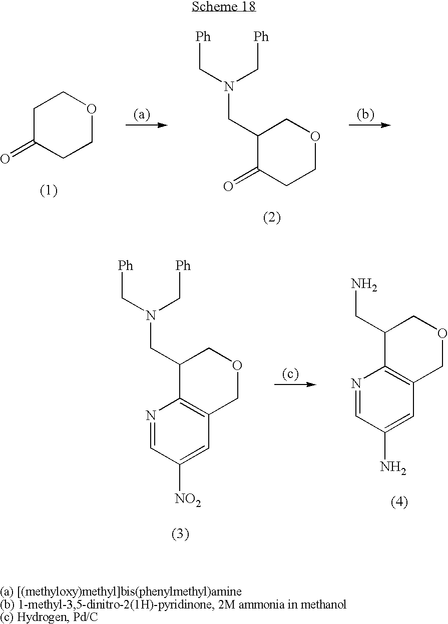

Reaction of ketone (1) with [(methyloxy)methyl]bis(phenylmethyl)amine affords tertiary amine (2) (see Ibrahem I. et al, Synthesis, 2006, 4060 for examples of this reaction). Reaction of (2) with 1-methyl-3,5-dinitro-2(1H)-pyridinone in methanolic ammonia gives nitropyridine (3) then hydrogenation gives diamine (4).

R³ groups may be interconverted by conventional methods such as those described above for R^{1a} and R^{1b}.

R⁴ groups may be interconverted by conventional methods, for example H may be converted to methyl by alkylation with methyl iodide in the presence of base.

Compounds of formulae (IIIA) and (IIIB) where R²⁰ is UR⁵ may be prepared from the corresponding compound of formula (IIIA) or (IIIB) where R²⁰ is hydrogen by the conventional means described above, or from a corresponding derivative where Q¹ and Q² are oxo which may be converted to (IIIA) or (IIIB) by reductive alkylation with an amine R⁵—CH₂—NH₂.

Compounds of formula (IIIA) where L is Br may be converted to the corresponding amine compound of formula (IIIB) by a palladium catalysed amination via benzophenone imine followed by hydrolysis to the primary amine (see Zhang, D. et al, Bioorganic & Medicinal Chemistry Letters (2004), 14(24), 6011-6016 for an example).

Further details for the preparation of compounds of formula (I) are found in the examples.

The antibacterial compounds according to the invention may be formulated for administration in any convenient way for use in human or veterinary medicine, by analogy with other antibacterials/antitubercular compounds.

The pharmaceutical compositions of the invention may be formulated for administration by any route and include those in a form adapted for oral, topical or parenteral use and may be used for the treatment of bacterial infection including tuberculosis in mammals including humans.

The composition may be in the form of tablets, capsules, powders, granules, lozenges, suppositories, creams or liquid preparations, such as oral or sterile parenteral solutions or suspensions.

The topical formulations of the present invention may be presented as, for instance, ointments, creams or lotions, eye ointments and eye or ear drops, impregnated dressings and aerosols, and may contain appropriate conventional additives such as preservatives, solvents to assist drug penetration and emollients in ointments and creams.

The formulations may also contain compatible conventional carriers, such as cream or ointment bases and ethanol or oleyl alcohol for lotions. Such carriers may be present as from about 1% up to about 98% of the formulation. More usually they will form up to about 80% of the formulation.

Tablets and capsules for oral administration may be in unit dose presentation form, and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavouring or colouring agents.

Suppositories will contain conventional suppository bases, e.g. cocoa-butter or other glyceride.

For parenteral administration, fluid unit dosage forms are prepared utilizing the compound and a sterile vehicle, water being preferred. The compound, depending on the vehicle and concentration used, can be either suspended or dissolved in the vehicle. In preparing solutions the compound can be dissolved in water for injection and filter sterilised before filling into a suitable vial or ampoule and sealing.

Advantageously, agents such as a local anaesthetic, preservative and buffering agents can be dissolved in the vehicle. To enhance the stability, the composition can be frozen after filling into the vial and the water removed under vacuum. The dry lyophilized powder is then sealed in the vial and an accompanying vial of water for injection may be supplied to reconstitute the liquid prior to use. Parenteral suspensions are prepared in substantially the same manner except that the compound is suspended in the vehicle instead of being dissolved and sterilization cannot be accomplished by filtration. The compound can be sterilised by exposure to ethylene oxide before suspending in the sterile vehicle. Advantageously, a surfactant or wetting agent is included in the composition to facilitate uniform distribution of the compound.

The compositions may contain from 0.1% by weight, preferably from 10-60% by weight, of the active material, depending on the method of administration. Where the compositions comprise dosage units, each unit will preferably contain from 50-1000 mg of the active ingredient. The dosage as employed for adult human treatment will preferably range from 100 to 3000 mg per day, for instance 1500 mg per day depending on the route and frequency of administration. Such a dosage corresponds to about 1.5 to about 50 mg/kg per day. Suitably the dosage is from 5 to 30 mg/kg per day.

The compound of formula (I) may be the sole therapeutic agent in the compositions of the invention or a combination with other antibacterials including antitubercular compounds. If the other antibacterial is a β-lactam then a β-lactamase inhibitor may also be employed.

Compounds of formula (I) may be used in the treatment of bacterial infections caused by a wide range of organisms including both Gram-negative and Gram-positive organisms, such as upper and/or lower respiratory tract infections, skin and soft tissue infections and/or urinary tract infections. Compounds of formula (I) may be also used in the treatment of tuberculosis caused by Mycobacterium tuberculosis. Some compounds of formula (I) may be active against more than one organism. This may be determined by the methods described herein.

The following examples illustrate the preparation of certain compounds of formula (I) and the activity of certain compounds of formula (I) against various bacterial organisms including Mycobacterium tuberculosis.

### EXAMPLES AND EXPERIMENTAL

### General

### Abbreviations in the Examples:

ES=Electrospray mass spec.
HPLC=High Performance Liquid Chromatography (Rt refers to retention time)
LCMS=Liquid chromatography mass spectroscopy
rt=room temperature
Rf=retention factor

Certain reagents are also abbreviated herein. DMF refers to dimethylformamide, DCM refers to dichloromethane, CHCl₃ refers to chloroform, DMSO refers to dimethylsulfoxide, EtOAc refers to ethyl acetate, MeOH refers to methanol, TFA refers to trifluoroacetic acid, THF refers to tetrahydrofuran, Et₂O refers to diethyl ether, Pd₂(dba)₃ refers to tris(dibenzylideneacetone)dipalladium(0), Pd/C refers to palladium on carbon catalyst and (±)-BINAP refers to 2,2′-bis(diphenylphosphino)-1,1′-binaphthyl. Proton nuclear magnetic resonance (¹H NMR) spectra were recorded at 250 or 400 MHz, and chemical shifts are reported in parts per million (6) downfield from the internal standard tetramethylsilane (TMS). Abbreviations for NMR data are as follows: s=singlet, d=doublet, t=triplet, q=quartet, m=multiplet, dd=doublet of doublets, dt=doublet of triplets, app=apparent, br=broad. J indicates the NMR coupling constant measured in Hertz. CDCl₃ is deuteriochloroform, DMSO-d₆ is hexadeuteriodimethylsulfoxide, and MeOD is tetradeuteriomethanol. Mass spectra were obtained using electrospray (ES) ionization techniques. All temperatures are reported in degrees Celsius.

MP-Carbonate resin is a commercially available macroporous polystyrene anion-exchange resin that is a resin-bound equivalent of tetraalkylammonium carbonate. MP-Carbonate may be used as a general base to neutralize amine hydrochlorides. Celite® is a filter aid composed of acid-washed diatomaceous silica, and is a trademark of Manville Corp., Denver, Colo.

The SCX (Strong Cation eXchange) column has benzene sulphonic acid covalently attached to a silica support and as such strongly retains high pKa (ie basic) organic molecules such as amines, which can be subsequently liberated with excess ammonia in an appropriate solvent.

Chiralpak AS-His a polysaccharide based chiral HPLC column (Chiral Technologies Inc.) comprising amylose tris [(S)— alpha-methylbenzylcarbamate) coated onto 5 um silica. Chiralpak AD-H columns comprise silica for preparative columns (5 um particle size AD-H, 21×250 mm) coated with Amylose tris (3,5-dimethylphenylcarbamate) (Chiral Technologies USA). Chiralpak IA column comprise silica for preparative column (5 um particle size, 21 mm ID×250 mm L) immobilized with Amylose tris (3,5-dimethylphenylcarbamate). Measured retention times are dependent on the precise conditions of the chromatographic procedures. Where quoted below in the Examples they are indicative of the order of elution.

Reactions involving metal hydrides including lithium hydride, lithium aluminium hydride, di-isobutylaluminium hydride, sodium hydride, sodium borohydride, sodium triacetoxyborohydride, (polystyrylmethyl)trimethylammonium cyanoborohydride are carried out under argon or other inert gas.

As will be understood by the skilled chemist, references to preparations carried out in a similar manner to, or by the general method of, other preparations, may encompass variations in routine parameters such as time, temperature, workup conditions, minor changes in reagent amounts etc.

### Example 1

### 1-{(6S)-6-[(2,3-Dihydro[1,4]dioxino[2,3-c]pyridin-7-ylmethyl)amino]-5,6,7,8-tetrahydro-2-naphthalenyl}-7-fluoro-2(1H)-quinolinone dihydrochloride

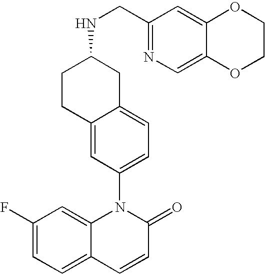

### (a) (2S)-6-Bromo-N-(2,3-dihydro[1,4]dioxino[2,3-c]pyridin-7-ylmethyl)-1,2,3,4-tetrahydro-2-naphthalenamine

(2S)-6-Bromo-1,2,3,4-tetrahydro-2-naphthalenamine (0.51 g, 1.94 mmol) was treated with CHCl₃ (20 mL) and MeOH (2 ml) under argon at room temperature and then triethylamine (0.541 ml, 3.88 mmol) was added. The reaction was stirred for 10 mins at room temperature when 2,3-dihydro[1,4]dioxino[2,3-c]pyridine-7-carbaldehyde (0.320 g, 1.94 mmol) (for a synthesis see WO2004058144, Example 2(c) or WO2003087098 Example 19(d)) was added followed by sodium triacetoxyborohydride (1.24 g, 5.83 mmol). The reaction was allowed to stir at room temperature for 16 h, after which the reaction was quenched by addition of aq. sat. NaHCO₃ solution (20 ml). The aqueous phase was then separated and then washed a further 3 times with 10% MeOH in DCM (10 ml). The organic layers were then combined, dried (Na₂SO₄), filtered and the solvent was removed to give a yellow oil. This residue was then purified using flash column chromatography eluting with 0-100% EtOAc in hexane then 0-20% MeOH in EtOAc gradient to give a white solid (0.707 g, 97%).

MS (ES+) m/z 376 (MH⁺).

### (b) 1,1-Dimethylethyl [(2S)-6-bromo-1,2,3,4-tetrahydro-2-naphthalenyl](2,3-dihydro[1,4]dioxin[2,3-c]pyridin-7-ylmethyl)carbamate

(2S)-6-Bromo-N-(2,3-dihydro[1,4]dioxin[2,3-c]pyridin-7-ylmethyl)-1,2,3,4-tetrahydro-2-naphthalenamine (0.707 g, 1.88 mmol) was dissolved in MeOH (10 ml) and NaHCO₃ (0.474 g, 5.65 mmol) was added at room temperature under argon. The reaction was stirred for 10 mins before it was cooled to 0° C., after which di-tert-butyl dicarbonate (0.452 g, 2.07 mmol) was added portionwise. The reaction was then allowed to warm to room temperature overnight. The reaction was then filtered, evaporated and the residue purified using flash column chromatography eluting with 0-100% EtOAc in hexane then 0-20% MeOH in EtOAc gradient to a clear oil (0.855 g, 96%).

MS (ES+) m/z 476 (MH⁺).

### (c) (2E)-N-(3-fluorophenyl)-3-phenyl-2-propenamide

Cinnamoyl chloride (3.6 g, 18 mmol) in ethyl acetate (14 mL) was added to a stirred mixture containing 3-fluoroaniline, ethyl acetate (28 mL) and saturated NaHCO₃ solution (28 mL) and ice (15 g) and stirred for 2 h. The organic layer was then separated and washed with 1N HCl then saturated brine and dried. Chromatography on silica gel eluting with 40% ethyl acetate/40-60 petroleum ether gave a white solid (4.14 g, 95%).

MS (ES+) m/z 242 (MH⁺).

### (d) Mixture of 7-fluoro-2(1H)-quinolinone and 5-fluoro-2(1H)-quinolinone

(2E)-N-(3-Fluorophenyl)-3-phenyl-2-propenamide (3.82 g, 15.8 mmol) in chlorobenzene (25 mL) was treated with aluminium trichloride (10.6 g, 79 mmol) portionwise over a 10 min period. The mixture was then heated to 125° C. for 3 h. The mixture was allowed to cool slightly then poured onto ice/water (ca 200 mL) then extracted with 10% methanol/ethyl acetate (2×250 mL) and dried, filtered and evaporated to a small volume giving a pink solid which was filtered off and then recrystallised from ethyl acetate/methanol to give the title 7-fluoro isomer (1.15 g, 44%).

¹H NMR δ(DMSO-d₆) 6.45 (1H, d), 6.97-7.07 (1H, m), 7.70-7.77 (1H, m), 7.91 (1H, d), 11.75-11.91 (1H, br s).

The mother liquors were evaporated to dryness to give a 1:1 mixture of the title 7-fluoro and 5-fluoro isomers (1 g, 39%).

### (e) 1,1-Dimethylethyl (2,3-dihydro[1,4]dioxino[2,3-c]pyridin-7-ylmethyl)[(2S)-6-(7-fluoro-2-oxo-1(2H)-quinolinyl)-1,2,3,4-tetrahydro-2-naphthalenyl]carbamate

1,1-Dimethylethyl [(2S)-6-bromo-1,2,3,4-tetrahydro-2-naphthalenyl](2,3-dihydro[1,4]dioxino[2,3-c]pyridin-7-ylmethyl)carbamate (0.855 g, 1.79 mmol), 7-fluoro-2(1H)-quinolinone (0.245 g, 1.49 mmol) and copper (I) iodide (0.057 g, 0.3 mmol) were suspended in 1,4-dioxane (8 ml) at room temperature under argon. This was degassed several times whereupon N,N′-dimethylethylenediamine (0.064 ml, 0.6 mmol) was added followed by K₃PO₄ (0.637 g, 3.0 mmol). The reaction was then heated to 110° C. overnight. Further copper (I) iodide (0.057 g, 0.3 mmol) and N,N′-dimethylethylenediamine (0.064 ml, 0.6 mmol) were added and the reaction was again left at 110° C. overnight, after which the reaction mixture was cooled, diluted with H₂O (5 ml) and EtOAc (5 ml). The aqueous phase was separated and washed a further 3 times with EtOAc (5 ml). The organic layers were combined, dried (Na₂SO₄) and solvent removed. This residue was then purified using flash column chromatography eluting with 0-100% EtOAc in hexane then 0-20% MeOH in EtOAc gradient to give a mixture of desired product and 7-fluoro-2(1H)-quinolinone. This mixture was re-dissolved in DCM, washed with 2M NaOH, organic layer separated and solvent removed to give a yellow solid (0.1085 g, 13%).

MS (ES+) m/z 558 (MH⁺).

### (f) Title Compound

1,1-Dimethylethyl (2,3-dihydro[1,4]dioxino[2,3-c]pyridin-7-ylmethyl)[(2S)-6-(7-fluoro-2-oxo-1(2H)-quinolinyl)-1,2,3,4-tetrahydro-2-naphthalenyl]carbamate (0.108 g, 0.19 mmol) was dissolved in DCM (2 ml) at room temperature under argon then TFA (1 ml) was added dropwise. After 1 h the solvent was removed, the residue was re-dissolved in MeOH (2 ml) and DCM (2 ml) and MP-carbonate resin was added until pH 8 was attained. After 15 mins the reaction was then filtered and the resin washed further with MeOH and DCM. The combined filtrates were evaporated to give a yellow oil. This residue was then purified using flash column chromatography eluting with 0-30% MeOH in DCM affording the free base of the title compound as clear oil.

MS (ES+) m/z 458 (MH⁺).

¹H NMR (250 MHz) δ(MeOD) 1.71-2.15 (1H, m), 2.69 (1H, br s), 2.71-3.09 (4H, m), 3.10-3.24 (1H, m), 3.90 (1H, s), 4.28-4.38 (4H, m), 6.32-6.43 (1H, m), 6.67 (1H, d), 6.98-7.09 (4H, m), 7.26-7.38 (1H, m), 7.74-7.80 (1H, m) and 7.99-8.03 (2H, m).

The free base of the title compound was converted to the title dihydrochloride salt by dissolving the free base in MeOH (2 ml), adding 1M HCl in MeOH (0.3 ml) and then removing the solvent to give the title compound (0.045 g).

### Example 21

### 1-{(6R)-6-[(2,3-Dihydro[1,4]dioxino[2,3-c]pyridin-7-ylmethyl)amino]-5,6,7,8-tetrahydro-2-naphthalenyl}-7-fluoro-2(1H)-quinolinone dihydrochloride

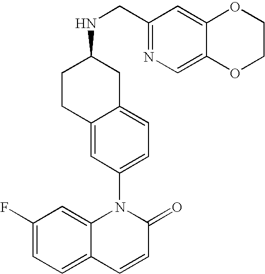

The title compound was prepared using (2R)-6-bromo-1,2,3,4-tetrahydro-2-naphthalenamine in a similar manner to Example 1 and the free base exhibited the same spectroscopic properties as for 1-{(65)-6-[(2,3-dihydro[1,4]dioxino[2,3-c]pyridin-7-ylmethyl)amino]-5,6,7,8-tetrahydro-2-naphthalenyl}-7-fluoro-2(1H)-quinolinone.

### Examples 3 and 4

### 4-{6-[(2,3-Dihydro[1,4]dioxino[2,3-c]pyridin-7-ylmethyl)amino]-5,6,7,8-tetrahydro-2-naphthalenyl}-6-(methyloxy)pyrido[2,3-b]pyrazin-3(4H)-one benzoate Enantiomers 1 and 2

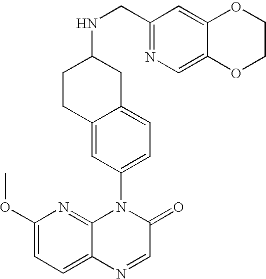

### (a) 7-(Azidomethyl)-2,3-dihydro[1,4]dioxino[2,3-c]pyridine

A solution of 2,3-dihydro[1,4]dioxino[2,3-c]pyridin-7-ylmethanol (for a synthesis see WO2004002490, Example 6(b)) (2.17 g, 12.99 mmol) and 1,8-diazabicyclo[5.4.0]undec-7-ene (2.13 ml, 14.29 mmol) in toluene (20 ml) was cooled to 0° C. and treated with diphenyl phosphoryl azide (3.35 ml, 15.59 mmol) and the reaction was allowed warm to room temperature and stirred at room temperature for 3 h before dichloromethane (500 ml) and aqueous sodium bicarbonate (500 ml) was added. The organic phase was separated and washed twice with water (2×500 ml). The organic phase was dried and evaporated. Chromatography of the residue on silica gel (methanol/dichloromethane gradient) gave the desired product (2.45 g, 98%).

### (b) 1-(2,3-dihydro[1,4]dioxino[2,3-c]pyridin-7-yl)methanamine

A mixture of 7-(azidomethyl)-2,3-dihydro[1,4]dioxino[2,3-c]pyridine (2.45 g, 12.76 mmol) and 10% palladium on carbon (250 mg) in methanol (100 ml) was stirred at room temperature over one atmosphere of hydrogen for 2 h. The mixture was filtered through Celite® and evaporated. Chromatography of the residue on silica gel (2M NH₃ in methanol/dichloromethane gradient) gave the desired product (1.11 g, 52%).

MS (ES+) m/z 167 (MH+).

### (c) (6-Bromo-1,2,3,4-tetrahydro-2-naphthalenyl)(2,3-dihydro[1,4]dioxino[2,3-c]pyridin-7-ylmethyl)amine

6-Bromo-2-tetralone (5.0 g, 22.1 mmol) (commercially available from Sigma-Aldrich) was dissolved in CHCl₃ (200 mL) and MeOH (20 ml) under argon at room temperature, then 1-(2,3-dihydro[1,4]dioxino[2,3-c]pyridin-7-yl)methanamine (3.7 g, 22.1 mmol) and sodium triacetoxyborohydride (0.117 g, 0.552 mmol) were added and the reaction was allowed to stir at room temperature for 16 h, after which the reaction was quenched by addition of aq. sat. NaHCO₃ solution (50 ml). The aqueous phase was separated and then washed a further 3 times with 10% MeOH in DCM. The organics were combined, dried (Na₂SO₄), filtered and the solvent was removed to give a black oil. This was then purified using flash column chromatography eluting with 0-100% EtOAc in hexane then 0-20% MeOH in EtOAc gradient to give a yellow oil (4.49 g, 54%).

MS (ES+) m/z 376 (MH⁺).

### (d) 1,1-Dimethylethyl (6-bromo-1,2,3,4-tetrahydro-2-naphthalenyl)(2,3-dihydro[1,4]dioxino[2,3-c]pyridin-7-ylmethyl)carbamate

(6-Bromo-1,2,3,4-tetrahydro-2-naphthalenyl)(2,3-dihydro[1,4]dioxino[2,3-c]pyridin-7-ylmethyl)amine (4.49 g, 11.9 mmol) was dissolved in MeOH (50 ml) and NaHCO₃ (3.02 g, 35.9 mmol) was added at room temperature under argon. The reaction was stirred for 10 mins before it was cooled to 0° C., after which di-tert-butyl dicarbonate (2.88 g, 13.2 mmol) was added portionwise. The reaction was then allowed to warm to room temperature overnight. The reaction was filtered, solvent removed and the residue purified using flash column chromatography eluting with 0-100% EtOAc in hexane then 0-20% MeOH in EtOAc gradient to give the title compound as a grey foam (4.91 g, 86%).

MS (ES+) m/z 476 (MH⁺).

### (e) 1,1-Dimethylethyl (2,3-dihydro[1,4]dioxino[2,3-c]pyridin-7-ylmethyl){6-[(diphenylmethylidene)amino]-1,2,3,4-tetrahydro-2-naphthalenyl}carbamate

1,1-Dimethylethyl (6-bromo-1,2,3,4-tetrahydro-2-naphthalenyl)(2,3-dihydro[1,4]dioxino[2,3-c]pyridin-7-ylmethyl)carbamate (4.69 g, 9.86 mmol), benzophenone imine (1.98 ml, 11.8 mmol), Pd₂(dba)₃ (0.023 g, 0.25 mol %), (±)-BINAP (0.046 g, 0.75 mol %) and sodium tert-butoxide (1.33 g, 13.8 mmol) were placed in reaction vessel under argon at room temperature. This flask was then evacuated and flushed with argon 3 times before dry toluene (100 ml) was added. The reaction was then heated to 80° C. overnight. More Pd₂(dba)₃ (0.023 g, 0.25 mol %), (±)-BINAP (0.046 g, 0.75 mol %) and sodium tert-butoxide (0.66 g, 6.9 mmol) were added and the mixture heated to 100° C. overnight. The reaction was then cooled, filtered and diluted with EtOAc (100 ml) and H₂O (100 ml). The aqueous layer was separated and washed a further 3 times with EtOAc (100 ml). The organics were combined, dried (Na₂SO₄), filtered and solvent removed. The residue was then purified using flash column chromatography eluting with 0-100% EtOAc in hexane then 0-20% MeOH in EtOAc gradient to give the title compound as a yellow solid (5.61 g, 99%).

MS (ES+) m/z 576 (MH⁺).

### (f) 1,1-Dimethylethyl (6-amino-1,2,3,4-tetrahydro-2-naphthalenyl)(2,3-dihydro[1,4]dioxino[2,3-c]pyridin-7-ylmethyl)carbamate

1,1-Dimethylethyl (2,3-dihydro[1,4]dioxino[2,3-c]pyridin-7-ylmethyl){6-[(diphenylmethylidene)amino]-1,2,3,4-tetrahydro-2-naphthalenyl}carbamate (5.61 g, 9.74 mmol) was dissolved in THF (100 ml) at room temperature under argon where 2M HCl (5 ml) was added. After 1 h reaction was quenched with aq. sat. NaHCO₃ solution (100 ml i.e. until pH9 was attained). The aqueous layer was separated and washed a further 3 times with 10% MeOH in DCM (50 ml). The organics were combined, dried (Na₂SO₄), filtered and solvent removed. The residue was then purified using flash column chromatography eluting with 0-100% EtOAc in hexane then 0-20% MeOH in EtOAc gradient to give a yellow solid (2.76 g, 69%).

MS (ES+) m/z 412 (MH⁺).

### (g) 1,1-Dimethylethyl (2,3-dihydro[1,4]dioxino[2,3-c]pyridin-7-ylmethyl)(6-{[6-(methyloxy)-3-nitro-2-pyridinyl]amino}-1,2,3,4-tetrahydro-2-naphthalenyl)carbamate

1,1-Dimethylethyl (6-amino-1,2,3,4-tetrahydro-2-naphthalenyl)(2,3-dihydro[1,4]dioxino[2,3-c]pyridin-7-ylmethyl)carbamate (2.76 g, 6.72 mmol) was dissolved in DMF (20 ml) at room temperature under argon where 2-chloro-6-methoxy-3-nitropyridine (1.27 g, 6.72 mmol) and NaHCO₃ (1.13 g, 13.4 mmol) were added. The reaction was then heated to 50° C., after 5 h the reaction was cooled to room temperature. The reaction was diluted with H₂O (40 ml) and DCM (40 ml). The aqueous layer was separated and washed a further 3 times with DCM (40 ml). The organics were combined, dried (Na₂SO₄), filtered and solvent removed. The residue was then purified using flash column chromatography eluting with 0-100% EtOAc in hexane then 0-20% MeOH in EtOAc gradient to give a yellow/orange solid (3.15 g, 83%).

MS (ES+) m/z 564 (MH⁺).

### (h) 1,1-Dimethylethyl (6-{[3-amino-6-(methyloxy)-2-pyridinyl]amino}-1,2,3,4-tetrahydro-2-naphthalenyl)(2,3-dihydro[1,4]dioxino[2,3-c]pyridin-7-ylmethyl)carbamate

1,1-Dimethylethyl (2,3-dihydro[1,4]dioxino[2,3-c]pyridin-7-ylmethyl)(6-{[6-(methyloxy)-3-nitro-2-pyridinyl]amino}-1,2,3,4-tetrahydro-2-naphthalenyl)carbamate (3.15 g, 5.58 mmol) was dissolved in EtOH (300 ml) at room temperature under argon where 10% Pd/C (1 g) was added. The reaction was then placed under an atmosphere of H₂ at room temperature overnight. The reaction was then filtered and residues wash further with EtOH. The organic layer was collected and the solvent removed to give a dark oil (3.35 g).

MS (ES+) m/z 534 (MH⁺).

### (i) Ethyl N-[2-{[6-((2,3-dihydro[1,4]dioxino[2,3-c]pyridin-7-ylmethyl){[(1,1-dimethylethyl)oxy]carbonyl}amino)-5,6,7,8-tetrahydro-2-naphthalenyl]amino}-6-(methyloxy)-3-pyridinyl]glycinate

1,1-Dimethylethyl (6-{[3-amino-6-(methyloxy)-2-pyridinyl]amino}-1,2,3,4-tetrahydro-2-naphthalenyl)(2,3-dihydro[1,4]dioxino[2,3-c]pyridin-7-ylmethyl)carbamate (2.98 g) was dissolved in DMF (50 mL) and acetonitrile (50 ml) under argon at room temperature, then K₂CO₃ (1.54 g, 11.1 mmol) and ethyl bromoacetate (0.619 ml, 5.58 mmol) was then added and the reaction was stirred at room temperature for 2 days. The reaction was diluted with H₂O (50 ml) and EtOAc (50 ml). The aqueous layer was separated and washed a further 3 times with EtOAc (50 ml). The organic extracts were combined, dried (Na₂SO₄), filtered and solvent removed. The residue was then purified using flash column chromatography eluting with 0-100% EtOAc in hexane then 0-20% MeOH in EtOAc gradient to give a yellow solid (2.68 g, 78%).

MS (ES+) m/z 620 (MH⁺).

### (j) 1,1-Dimethylethyl (2,3-dihydro[1,4]dioxino[2,3-c]pyridin-7-ylmethyl) {6-[6-(methyloxy)-3-oxo-2,3-dihydropyrido[2,3-b]pyrazin-4(1H)-yl]-1,2,3,4-tetrahydro-2-naphthalenyl}carbamate

Ethyl N-[2-{[6-((2,3-dihydro[1,4]dioxin[2,3-c]pyridin-7-ylmethyl) {[(1,1-dimethylethyl)oxy]carbonyl}amino)-5,6,7,8-tetrahydro-2-naphthalenyl]amino}-6-(methyloxy)-3-pyridinyl]glycinate (1 g, 1.61 mmol) was dissolved in THF (60 mL) at room temperature under argon. The reaction was then cooled to 0° C. and 1M potassium tert-butoxide in THF (0.807 ml, 0.807 mmol) was added dropwise. The reaction was then quenched with aq. sat. NH₄Cl solution (20 ml). The aqueous layer was separated and washed a further 4 times with EtOAc (50 ml). The organics were combined, dried (Na₂SO₄), filtered and solvent removed. The residue was then purified using flash column chromatography eluting with 0-100% EtOAc in hexane then 0-20% MeOH in EtOAc gradient to give a brown oil (0.599 g, 65%).

MS (ES+) m/z 574 (MH⁺).

### (k) 1,1-Dimethylethyl (2,3-dihydro[1,4]dioxino[2,3-c]pyridin-7-ylmethyl){6-[6-(methyloxy)-3-oxopyrido[2,3-b]pyrazin-4(3H)-yl]-1,2,3,4-tetrahydro-2-naphthalenyl}carbamate

1,1-Dimethylethyl (2,3-dihydro[1,4]dioxino[2,3-c]pyridin-7-ylmethyl){6-[6-(methyloxy)-3-oxo-2,3-dihydropyrido[2,3-b]pyrazin-4(1H)-yl]-1,2,3,4-tetrahydro-2-naphthalenyl}carbamate (0.599 g, 1.04 mmol) was dissolved in DCM (50 ml) at room temperature under argon where manganese (II) oxide (1.36 g, 15.7 mmol) was added. After 1 h the reaction was filtered and the residue washed further with DCM. The filtrate was collected and the solvent removed to give a black oil. This residue was then purified using flash column chromatography eluting with 0-100% EtOAc in hexane then 0-20% MeOH in EtOAc gradient to give the title compound as a brown oil (0.463 g, 78%).

MS (ES+) m/z 572 (MH⁺).

### (l) 4-{6-[(2,3-Dihydro[1,4]dioxino[2,3-c]pyridin-7-ylmethyl)amino]-5,6,7,8-tetrahydro-2-naphthalenyl}-6-(methyloxy)pyrido[2,3-b]pyrazin-3(4H)-one

1,1-Dimethylethyl (2,3-dihydro[1,4]dioxino[2,3-c]pyridin-7-ylmethyl){6-[6-(methyloxy)-3-oxopyrido[2,3-b]pyrazin-4(3H)-yl]-1,2,3,4-tetrahydro-2-naphthalenyl}carbamate (0.463 g, 0.809 mmol) was dissolved in DCM (3 ml) at room temperature under argon where TFA (2 ml) was added dropwise. After 30 mins the solvent was removed, this residue was re-dissolved in 1:1 mix MeOH and DCM (30 ml) and MP-carbonate resin was added until pH8 was attained. The mixture was then filtered and the resin washed further with MeOH. The combined filtrates were evaporated to give the free base of the racemate of the title compound as a white solid (0.320 g, 84%).

MS (ES+) m/z 472 (MH⁺).

¹H NMR (250 MHz) δ(MeOD) 1.95-1.92 (1H, m), 2.15-2.20 (1H, m), 2.66-3.06 (4H, m), 3.07-3.29 (1H, m), 3.61 (3H, s), 3.92 (2H, s), 4.27-4.39 (4H, m), 6.78 (1H, d), 7.02-7.07 (3H, m), 7.24-7.30 (1H, m) and 7.95-8.17 (3H, m).

A portion of the free base of the racemate of the title compound (0.247 g) was chromatographed on a Chiralpak AD-H column eluting with 90% acetonitrile: 10% methanol: 0.1% isopropylamine affording firstly the E1 enantiomer (0.125 g, Rt 7.1 mins) then the E2 enantiomer (0.115 g, Rt 11.4 mins). These materials were individually converted to the title benzoate salts by treatment of a solution with 1 equivalent of benzoic acid.

### Example 5

### 4-{6-[(6,7-Dihydro[1,4]dioxino[2,3-c]pyridazin-3-ylmethyl)amino]-4,5,6,7-tetrahydro-1,3-benzothiazol-2-yl}-6-(methyloxy)pyrido[2,3-b]pyrazin-3(4H)-one dihydrochloride

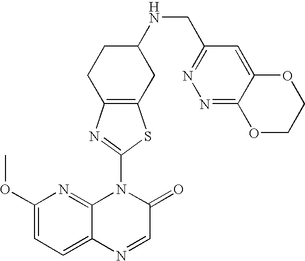

### (a) 1,1-Dimethylethyl (3-bromo-4-oxocyclohexyl)carbamate

A solution of 1,1-dimethylethyl (4-oxocyclohexyl)carbamate (2 g, 9.4 mmol) in ethyl acetate (100 mL) was treated with aluminium trichloride (38 mg, 0.28 mmol) to give a cloudy mixture. Stirring was stopped and bromine (1 drop) was added at 0° C. After 5 minutes bromine (0.47 mL, 9.3 mmol) was added. Decolourisation occurred within 10 minutes then the mixture was added to ethyl acetate (50 mL) and 2% aqueous sodium sulphate. The aqueous phase was further extracted with ethyl acetate and the combined organic extracts were washed with dilute aqueous sodium bicarbonate solution, dried and evaporated affording the product (2.3 g, 85%).

MS (ES+) m/z 293, 295 (MH⁺).

### (b) 1,1-Dimethylethyl (2-amino-4,5,6,7-tetrahydro-1,3-benzothiazol-6-yl)carbamate

A solution of 1,1-dimethylethyl (3-bromo-4-oxocyclohexyl)carbamate (1 g, 3.4 mmol) in acetonitrile (34 mL) was treated with diisopropylethylamine (1.2 mL, 6.9 mmol) then thiourea (0.26 g, 3.4 mmol). The mixture was heated to reflux for 15 minutes then evaporated to dryness. The residue was chromatographed eluting with 5-25% methanol in DCM affording the product (0.82 g, 89%).

MS (ES+) m/z 270 (MH⁺).

### (c) 1,1-Dimethylethyl (2-{[6-(methyloxy)-3-nitro-2-pyridinyl]amino}-4,5,6,7-tetrahydro-1,3-benzothiazol-6-yl)carbamate

A solution of 1,1-dimethylethyl (2-amino-4,5,6,7-tetrahydro-1,3-benzothiazol-6-yl)carbamate (1 g, 3.7 mmol) in THF (20 mL) was added to sodium hydride (60% dispersion with mineral oil, 4.1 mmol). After 20 minutes a solution of 2-chloro-6-methoxy-3-nitropyridine (0.7 g, 3.7 mmol) in THF (10 mL) was added. After 2 hours the mixture was treated with saturated aqueous ammonium chloride (20 mL) and extracted with ethyl acetate (3×30 mL). The combined organic extracts were dried and evaporated (1.7 g). The residue was chromatographed eluting with 0-100% ethyl acetate in hexane affording a yellow solid (0.55 g, 35%).

MS (ES+) m/z 422 (MH⁺).

### (d) 1,1-Dimethylethyl (2-{[3-amino-6-(methyloxy)-2-pyridinyl]amino}-4,5,6,7-tetrahydro-1,3-benzothiazol-6-yl)carbamate

A solution of 1,1-dimethylethyl (2-{[6-(methyloxy)-3-nitro-2-pyridinyl]amino}-4,5,6,7-tetrahydro-1,3-benzothiazol-6-yl)carbamate (0.55 g, 1.3 mmol) in ethanol (100 mL) was treated at 60° C. with ammonium formate (0.52 g, 8.3 mmol) and palladium on charcoal (0.2 g). After 1 hour the mixture was cooled, filtered and evaporated. The process was repeated with ammonium formate (0.9 g), palladium on charcoal (0.3 g) and at 70° C. After 3 hours the mixture was cooled, filtered, washing with ethanol and evaporated affording a purple solid (0.47 g, 92%).

MS (ES+) m/z 392 (MH⁺).

### (e) Ethyl (2E)-{[2-{[6-({[(1,1-dimethylethyl)oxy]carbonyl}amino)-4,5,6,7-tetrahydro-1,3-benzothiazol-2-yl]amino}-6-(methyloxy)-3-pyridinyl]imino}ethanoate

A solution of 1,1-dimethylethyl (2-{[3-amino-6-(methyloxy)-2-pyridinyl]amino}-4,5,6,7-tetrahydro-1,3-benzothiazol-6-yl)carbamate (50 mg, 0.13 mmol) in toluene (2.3 mL) was treated with a 50% solution of ethyl oxoacetate in toluene (0.025 mL, 1.5 mmol). After 3 hours the mixture was concentrated. Chromatography eluting with 20-100% ethyl acetate in hexane afforded a yellow solid (0.02 g, 33%).

MS (ES+) m/z 476 (MH⁺).

### (f) 1,1-Dimethylethyl {2-[6-(methyloxy)-3-oxopyrido[2,3-b]pyrazin-4(3H)-yl]-4,5,6,7-tetrahydro-1,3-benzothiazol-6-yl}carbamate

A solution of ethyl (2E)-{[2-{[6-({[(1,1-dimethylethyl)oxy]carbonyl}amino)-4,5,6,7-tetrahydro-1,3-benzothiazol-2-yl]amino}-6-(methyloxy)-3-pyridinyl]imino}ethanoate (20 mg, 0.04 mmol) in THF (2 mL), was treated with a 1M solution of potassium t-butoxide in THF (1 drop) and stirred overnight under argon. The mixture was treated with saturated aqueous ammonium chloride (5 mL) and extracted with ethyl acetate (5 mL). The combined organic extracts were dried and evaporated. The residue was chromatographed eluting with 50% ethyl acetate in hexane affording a yellow oil (17 mg, 100%).

MS (ES+) m/z 430 (MH⁺).

### (g) 4-(6-Amino-4,5,6,7-tetrahydro-1,3-benzothiazol-2-yl)-6-(methyloxy)pyrido[2,3-b]pyrazin-3(4H)-one

A solution of 1,1-dimethylethyl {2-[6-(methyloxy)-3-oxopyrido[2,3-b]pyrazin-4(3H)-yl]-4,5,6,7-tetrahydro-1,3-benzothiazol-6-yl}carbamate (150 mg, 0.35 mmol) in DCM (6 mL) was treated with 4M HCl in 1,4-dioxane (0.09 mL, ca 1 equivalent). After 3.5 hours more 4M HCl in 1,4-dioxane (0.9 mL, ca 10 equivalents) was added. After 3 hours the mixture was evaporated, azeotroping with DCM/methanol (1/1, 2×20 mL). This material was purified by chromatography on SCX, eluting with DCM/methanol (1/1), then an ammonia in methanol gradient affording a brown oil (111 mg, 95%).

MS (ES+) m/z 330 (MH⁺).

### (h) 3,4,6-Trichloropyridazine

This was prepared by a slight variation on the method of Kasnar et al, Nucleosides & Nucleotides (1994), 13(1-3), 459-79.

Hydrazine sulphate salt (51 g) was suspended in water (250 ml), heated to reflux and bromomaleic anhydride (90.38 g) was added dropwise. The mixture was heated at reflux for 4 hours then cooled to room temperature. The reaction was repeated with 29 g hydrazine sulphate, 53 g bromomaleic anhydride and 130 ml water. The precipitates were collected by filtration, washed with water and acetone and dried as a combined batch in vacuo to afford 4-bromo-1,2-dihydro-3,6-pyridazinedione as a white solid (113 g).

The solid in two batches was treated with phosphorus oxychloride (2×200 ml) and heated to reflux for 3.5 hours. The mixture was cooled, evaporated and azeotroped with toluene. The residue was partitioned between dichloromethane and saturated aqueous sodium bicarbonate solution and extracted with DCM twice more. The organic extracts were dried and evaporated. This residue was re-dissolved in dichloromethane, and chromatographed on silica gel (300 g) (DCM as eluent) to give a white solid (101.5 g, 87%).

(LC/MS analysis showed ca 20-30% impurity, isomers of bromo-dichloropyridazine).

MS (+ve ion electrospray) m/z 184/185/186 (MH⁺), trichloropyridazine.

MS (+ve ion electrospray) m/z 228/229/231 (MH⁺), bromo-dichloropyridazine.

### (i) 2-[(3,6-Dichloro-4-pyridazinyl)oxy]ethanol

A solution of ethylene glycol (55 ml) in tetrahydrofuran (200 ml) was treated at around 0° C. (ice bath cooling) with sodium hydride (60% dispersion in oil, 5.9 g) over 40 minutes. After the addition was complete, 3,4,6-trichloropyridazine containing isomers of bromo-dichloropyridazine as impurity (27 g) was added portionwise and washed in with more dry THF (50 ml) and the mixture was stirred at 0° C. for 1 hour then at room temperature overnight. The mixture was concentrated (to ⅓ volume) then diluted with aqueous sodium bicarbonate solution and extracted with chloroform (5×) and ethyl acetate (3×). The combined organic extracts were washed with water, dried over sodium sulphate and evaporated and the solid filtered off and washed with CHCl₃ (×3) and dried in a vacuum over overnight at 40° C. affording a white solid (25.5 g, 83%), containing some bromo-derivative (10-15%).

MS (+ve ion electrospray) m/z 209/211 (MH⁺).

MS (+ve ion electrospray) m/z 255/7 (MH⁺), bromo-derivative.

### (j) 3-Chloro-6,7-dihydro[1,4]dioxino[2,3-c]pyridazine

A solution of 2-[(3,6-dichloro-4-pyridazinyl)oxy]ethanol containing some bromo-derivative (15.46 g; 0.0703 mol) in dry 1,4-dioxane (1.2 L) was treated with lithium hydride (2.3 g; 0.28 mol) in portions and stirred at room temperature for 1 hour under argon, then heated at 110° C. overnight. The reaction mixture was quenched with wet 1,4-dioxane, then iced-water. The solution was evaporated to half volume, taken to pH 8 with 5M hydrochloric acid and evaporated to dryness. Water was added and the residue was extracted 5× with chloroform, dried (sodium sulphate) and evaporated to afford a white solid (12.4 g, ca.77%) (containing ca. 15% of a bromo species).

MS (+ve ion electrospray) m/z 173/5 (Cl MH+); 217/9 (Br MH+).

### (k) 3-Ethenyl-6,7-dihydro[1,4]dioxino[2,3-c]pyridazine

A solution of 3-chloro-6,7-dihydro[1,4]dioxino[2,3-c]pyridazine containing ca. 15% of a bromo species (13.6 g, 0.079 mol) in dimethoxyethane (400 ml) was degassed under argon for 10 min then tetrakis(triphenylphosphine)palladium (0) (2 g), potassium carbonate (10.33 g), 2,4,6-trivinylcyclotriboroxane pyridine complex (11.32 g) and water (55 ml) were added. The mixture was heated at 95° C. for 48 hours and cooled and evaporated to dryness. The mixture was treated with aqueous sodium bicarbonate solution and extracted (5×) with DCM. Extracts were dried (sodium sulphate), evaporated and the residue chromatographed on silica gel (500 g), eluting with 0-100% ethyl acetate—hexane, affording the product (6.43 g, 50%); [also some impure fractions (1.8 g)].

MS (+ve ion electrospray) m/z 165 (MH+).

### (1) 6,7-Dihydro[1,4]dioxino[2,3-c]pyridazine-3-carbaldehyde

A solution of 3-ethenyl-6,7-dihydro[1,4]dioxino[2,3-c]pyridazine (11.58 g) in 1,4-dioxane/water (600 ml/180 ml), cooled in ice, was treated with an aqueous solution of osmium tetroxide (4% w/v, 25 ml) and sodium periodate (43 g). This mixture was allowed to warm to room temperature and after 7 hours under stirring the mixture was evaporated to dryness and azeotroped with 1,4-dioxane. Silica gel, 1,4-dioxane and chloroform were added and the mixture was evaporated to dryness overnight, then added to a silica column (400 g) and chromatographed, eluting with chloroform then 0-100% ethyl acetate in hexane, to afford a white solid (7.55 g, 64%).

MS (+ve ion electrospray) m/z 167 (MH+).

### (m) Title Compound

A solution of 4-(6-amino-4,5,6,7-tetrahydro-1,3-benzothiazol-2-yl)-6-(methyloxy)pyrido[2,3-b]pyrazin-3(4H)-one (55 mg, 0.17 mmol) and 6,7-dihydro[1,4]dioxino[2,3-c]pyridazine-3-carbaldehyde (28 mg, 0.17 mmol) in DCM/methanol (20 mL/2.5 mL) was treated with sodium triacetoxyborohydride (ca 100 mg, ca 0.5 mmol). After stirring for 20 hours the dark mixture was treated with more sodium triacetoxyborohydride (ca 100 mg). After 2 hours acetic acid (3 drops) was added. More sodium triacetoxyborohydride (ca 100 mg) was added and the mixture stirred overnight. The mixture was concentrated (to ca 3 mL) then more 6,7-dihydro[1,4]dioxino[2,3-c]pyridazine-3-carbaldehyde (20 mg) and more sodium triacetoxyborohydride (ca 100 mg) were added. After 3 hours more 6,7-dihydro[1,4]dioxino[2,3-c]pyridazine-3-carbaldehyde (20 mg) and more sodium triacetoxyborohydride (ca 100 mg) were added. After 2 hours the mixture was partitioned between DCM and saturated aqueous sodium bicarbonate solution. The organic extract was dried and evaporated and the black residue chromatographed three times eluting with 0-50% methanol in DCM affording the free base of the title compound as a yellow oil (20 mg, 25%).

δH (CDCl₃, 250 MHz) 1.80-1.90 (1H, m), 2.15-2.25 (1H, m), 2.70-2.80 (1H, m), 2.85-3.05 (2H, m), 3.15-3.25 (2H, m), 3.78 (3H, s), 4.10 (2H, q), 4.35-4.40 (2H, m), 4.50-4.55 (2H, m), 6.75 (1H, d), 7.08 (1H, s), 8.08 (1H, d), 8.22 (1H, s).

MS (ES+) m/z 480 (MH⁺).

The free base of the title compound was dissolved in DCM and treated with excess HCl in ether to afford the title compound as a solid (25 mg).

### Example 6

### 6-[({3-[6-(Methyloxy)-3-oxopyrido[2,3-b]pyrazin-4(3H)-yl]-5,6,7,8-tetrahydro-7-quinolinyl}amino)methyl]-2H-pyrido[3,2-b][1,4]oxazin-3(4H)-one dihydrochloride

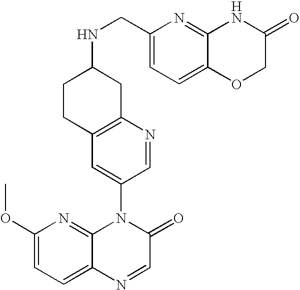

### (a) 1,1-Dimethylethyl (3-amino-5,6,7,8-tetrahydro-7-quinolinyl)carbamate

A solution of phenylmethyl (3-nitro-5,6,7,8-tetrahydro-7-quinolinyl)carbamate (28.6 g, 87.3 mmol) (for a synthesis see WO 2006/040178 Example 11.2) in ethanol (250 mL) at room temperature, was purged with argon then 10% palladium on charcoal paste (3 g) was added in one portion. The resulting suspension was evacuated and purged with argon three times, then subjected to an atmosphere of hydrogen and stirred vigorously at room temperature for 6 h. The reaction mixture was evacuated and purged with argon several times then stirred at room temperature for 15 h. The suspension was filtered through a pad of Celite® and the precipitate was washed extensively with ethanol The filtrate was concentrated to deliver an orange oil (16.9 g, >100%) which was used in the next step without further purification.

To a solution of this material (16.9 g) in methanol (350 mL) at 0° C., was added sodium hydrogen carbonate (22 g, 261.9 mmol) then di-tent-butyl dicarbonate (20.9 g, 96.0 mmol) was added. The reaction mixture was warmed to room temperature and after 2 h at room temperature. The reaction mixture was filtered through a pad of Celite then concentrated to deliver a sticky orange gum. The gum was suspended in 9:1 DCM:MeOH (100 mL) and filtered through a pad of silica. The filtrate was partially concentrated then placed directly onto a 400 g silica cartridge, eluting with 0-10% MeOH:DCM to deliver an orange amorphous solid (4.89 g, 21% over 2 steps).

MS (ES+) m/z 264 (MH⁺).

The original silica pad was suspended in 9:1 DCM:MeOH (100 mL) and stirred at room temperature for 18 h then filtered. The filtrate was concentrated to afford a dark orange amorphous solid (4.50 g, 20% over 2 steps).

MS (ES+) m/z 264 (MH⁺).

### (b) 1,1-Dimethylethyl (3-{[6-(methyloxy)-3-nitro-2-pyridinyl]amino}-5,6,7,8-tetrahydro-7-quinolinyl)carbamate

To a solution of 1,1-dimethylethyl (3-amino-5,6,7,8-tetrahydro-7-quinolinyl)carbamate (4.89 g, 18.6 mmol) in DMF (60 mL) at room temperature under argon was added 2-chloro-6-(methyloxy)-3-nitropyridine (3.50 g, 18.6 mmol) then sodium hydrogen carbonate (3.12 g, 37.2 mmol). The resulting reaction mixture was heated at 60° C. overnight then cooled to room temperature and concentrated. The residue was partitioned between DCM and H₂O then separated. The aqueous layer was extracted several times with DCM and the combined organic extracts were washed with brine, dried over MgSO₄, filtered and concentrated to deliver an orange oil. This residue was divided into two equal portions, and each portion was purified by flash column chromatography eluting with 0-100% EtOAc:Hexane to afford a yellow solid [5.51 g (combined mass), 71%].

MS (ES+) m/z 416 (MH⁺).

### (c) Ethyl N-[2-{[7-({[(1,1-dimethylethyl)oxy]carbonyl}amino)-5,6,7,8-tetrahydro-3-quinolinyl]amino}-6-(methyloxy)-3-pyridinyl]glycinate

To a solution of 1,1-dimethylethyl (3-{[6-(methyloxy)-3-nitro-2-pyridinyl]amino}-5,6,7,8-tetrahydro-7-quinolinyl)carbamate (2.08 g, 5.01 mmol) in ethanol (100 mL) at room temperature, was purged with argon then 10% palladium on charcoal paste (1 g) was added in one portion. The resulting suspension was evacuated and purged with argon three times, then subjected to an atmosphere of hydrogen and stirred vigorously at room temperature for 2 h. The reaction mixture was evacuated and purged with argon several times. The suspension was filtered through a pad of Celite® and the precipitate was washed extensively with ethanol The filtrate was concentrated to deliver a clear purple oil which was used in the next step without further purification.

To a solution of this material in toluene (50 mL) at room temperature was added ethyl glyoxylate (1.19 mL, 6.01 mmol, 50% solution in toluene) and the resulting dark green solution was stirred at room temperature overnight. Two 5 mL aliquots of the reaction mixture were removed for test reactions and the remaining reaction mixture (40 mL) was stirred at room temperature overnight. A further 5 mL of the reaction mixture was removed for a test reaction and the remaining reaction mixture was stirred at room temperature for a further four days. Methanol (35 mL) was added to the reaction mixture and the resulting solution was cooled to 0° C. Sodium borohydride powder (146 mg, 3.85 mmol) was added to the reaction mixture in one portion and vigorous gas evolution was observed. The mixture was then warmed to room temperature and stirred for 10 min. The reaction was quenched with H₂O and the layers were separated. The aqueous layer was extracted several times with ethyl acetate and the combined organic extracts were dried over MgSO₄, filtered and concentrated to deliver an orange oil. The residue was purified by chromatography eluting with 0-10% MeOH:DCM to afford a brown foam (1.48 g, 90% over 3 steps).

MS (ES+) m/z 472 (MH⁺).

### (d) 1,1-Dimethylethyl {3-[6-(methyloxy)-3-oxo-2,3-dihydropyrido[2,3-b]pyrazin-4(1H)-yl]-5,6,7,8-tetrahydro-7-quinolinyl}carbamate

To a solution of ethyl N-[2-{[7-({[(1,1-dimethylethyl)oxy]carbonyl}amino)-5,6,7,8-tetrahydro-3-quinolinyl]amino}-6-(methyloxy)-3-pyridinyl]glycinate in THF (60 mL) at 0° C. was added 1M potassium tert-butoxide in THF (3.46 mL, 3.46 mmol) dropwise. The resulting dark brown solution was stirred at 0° C. then warmed to room temperature. After a further 10 minutes at room temperature the reaction was quenched with aq. sat. NH₄Cl solution and the reaction mixture was concentrated. The residue was partitioned between aq. sat. NH₄Cl solution and ethyl acetate and the layers were separated. The aqueous layer was extracted a further 3 times with EtOAc. The combined organics were dried over MgSO₄, filtered and concentrated to deliver a dark brown oil. The residue was then purified using flash column chromatography on a 70 g silica cartridge eluting with 0-10% MeOH:DCM to afford a dark orange oil (1.22 g, 91%).

MS (ES+) m/z 426 (MH⁺).

### (e) 1,1-Dimethylethyl {3-[6-(methyloxy)-3-oxopyrido[2,3-b]pyrazin-4(3H)-yl]-5,6,7,8-tetrahydro-7-quinolinyl}carbamate

To a solution of 1,1-dimethylethyl {3-[6-(methyloxy)-3-oxo-2,3-dihydropyrido[2,3-b]pyrazin-4(1H)-yl]-5,6,7,8-tetrahydro-7-quinolinyl}carbamate (58 mg, 0.14 mmol) in DCM (10 mL) at room temperature was added MnO₂ (183 mg, 2.10 mmol) in one portion. The resulting suspension was stirred at room temperature for 1 h, filtered and the filtrate concentrated to deliver a brown oil. The residue was purified by flash column chromatography on a 5 g Flashmaster silica cartridge, eluting with 0-10% MeOH:DCM to afford a pale yellow clear oil (48 mg, 81%).

MS (ES+) m/z 424 (MH⁺).

### (f) 4-(7-Amino-5,6,7,8-tetrahydro-3-quinolinyl)-6-(methyloxy)pyrido[2,3-b]pyrazin-3(4H)-one

To a solution of 1,1-dimethylethyl {3-[6-(methyloxy)-3-oxopyrido[2,3-b]pyrazin-4(3H)-yl]-5,6,7,8-tetrahydro-7-quinolinyl}carbamate (752 mg, 1.80 mmol) in DCM (5 mL) at 0° C. was added TFA (5 mL) dropwise. The resulting dark brown solution was warmed to room temperature and stirred for 45 min before the reaction mixture was concentrated. The residue was azeotroped sequentially with chloroform then toluene to deliver a dark orange oil. This oil was dissolved in 9:1 DCM:MeOH (100 mL) and MP-carbonate resin was added until pH 8 was attained. After stirring at room temperature for 20 min the reaction was then filtered and concentrated to afford a light orange powder (435 mg, 75%).

MS (ES+) m/z 324 (MH⁺).

### (g) Title Compound

To a solution of 4-(7-amino-5,6,7,8-tetrahydro-3-quinolinyl)-6-(methyloxy)pyrido[2,3-b]pyrazin-3(4H)-one (145 mg, 0.45 mmol) in DCM (5 mL) and MeOH (0.5 mL) at room temperature was added 3-oxo-3,4-dihydro-2H-pyrido[3,2-b][1,4]oxazine-6-carbaldehyde (104 mg, 0.59 mmol) (for a synthesis see WO03087098 Example 31(e)) then sodium triacetoxyborohydride (286 mg, 1.35 mmol). The resulting suspension was stirred at room temperature for 2 h then the reaction mixture was diluted with DCM (10 mL) and washed with sat. aq. NaHCO₃ (10 mL). The aqueous was then separated and washed a further three times with DCM (10 mL). The combined organic layers were then dried over Na₂SO₄, filtered and concentrated to deliver an orange powder. This residue was then purified by flash column chromatography eluting with 0-10% MeOH:DCM to afford a clear orange oil (59 mg, 27%).

MS (ES+) m/z 486 (MH⁺).

¹H NMR (250 MHz) δ(CDCl₃) 1.77-1.86 (1H, m), 2.14-2.19 (1H, m), 2.45-3.04 (3H, m), 3.15-3.22 (1H, m), 3.36 (1H, dd,), 3.67 (3H, s), 3.99 (2H, s), 4.64 (2H, s), 6.74 (1H, d), 6.98 (1H, d), 7.22 (1H, d), 7.42 (1H, d), 8.08 (1H, d), 8.27 (1H, s), 8.39 (1H, d).

The free base of the title compound was converted to the HCl salt by dissolving the free base in MeOH, adding 1 M HCl in MeOH (0.18 ml) and then removing the solvent to give the dihydrochloride salt of the title compound (54 mg).

### Example 7

### 4-{7-[(6,7-Dihydro[1,4]dioxino[2,3-c]pyridazin-3-ylmethyl)amino]-5,6,7,8-tetrahydro-3-quinolinyl}-6-(methyloxy)pyrido[2,3-b]pyrazin-3(4H)-one hydrochloride

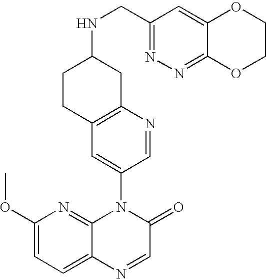

To a solution of 4-(7-amino-5,6,7,8-tetrahydro-3-quinolinyl)-6-(methyloxy)pyrido[2,3-b]pyrazin-3(4H)-one (for a preparation see Example 6(f)) (145 mg, 0.45 mmol) in DCM (5 mL) and MeOH (0.5 mL) at room temperature was added 6,7-dihydro[1,4]dioxino[2,3-c]pyridazine-3-carbaldehyde (for a preparation see Example 5(1)) (97 mg, 0.59 mmol) then sodium triacetoxyborohydride (286 mg, 1.35 mmol). The resulting suspension was stirred at room temperature for 48 h then the reaction mixture was diluted with DCM (10 mL) and washed with sat. aq. NaHCO₃ (10 mL). The aqueous was then separated and washed a further three times with DCM (10 mL). The combined organic layers were then dried over Na₂SO₄, filtered and concentrated to deliver an orange oil. This residue was then purified by flash column chromatography, eluting with 0-10% MeOH:DCM to afford the free base of the title compound as a clear orange oil (27 mg, 13%).

MS (ES+) m/z 474 (MH⁺).

¹H NMR (250 MHz) δ(CDCl₃) 1.68-1.84 (1H, m), 2.13-2.19 (1H, m), 2.82-3.04 (3H, m), 3.15-3.18 (1H, m), 3.23 (1H, dd), 3.67 (3H, s), 4.13 (2H, s), 4.36-4.40 (2H, m), 4.51-4.54 (2H, m), 6.75 (1H, d), 7.03 (1H, d), 7.42 (1H, d), 8.07 (1H, d), 8.27 (1H, s), 8.37 (1H, d).

The free base of the title compound was converted to the HCl salt by dissolving the free base in MeOH, adding 1 M HCl in MeOH (0.09 ml) and then removing the solvent to give the hydrochloride salt of the title compound (26 mg).

### Example 8

### 4-{7-[(2,3-Dihydro[1,4]dioxino[2,3-c]pyridin-7-ylmethyl)amino]-5,6,7,8-tetrahydro-3-quinolinyl}-6-(methyloxy)pyrido[2,3-b]pyrazin-3(4H)-one hydrochloride

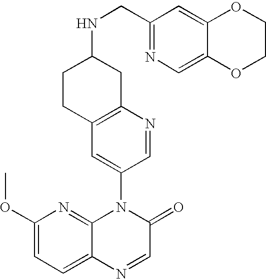

To a solution of 4-(7-amino-5,6,7,8-tetrahydro-3-quinolinyl)-6-(methyloxy)pyrido[2,3-b]pyrazin-3(4H)-one (for a preparation see Example 6(f)) (145 mg, 0.45 mmol) in DCM (5 mL) and MeOH (0.5 mL) at room temperature was added 2,3-dihydro[1,4]dioxino[2,3-c]pyridine-7-carbaldehyde (97 mg, 0.59 mmol) (for a synthesis see WO2004058144, Example 2(c) or WO2003087098 Example 19(d)) then sodium triacetoxyborohydride (286 mg, 1.35 mmol). The resulting suspension was stirred at room temperature for 1 h then the reaction mixture was diluted with DCM (10 mL) and washed with sat. aq. NaHCO₃ (10 mL). The aqueous was then separated and washed a further three times with DCM (10 mL). The combined organic layers were then dried over Na₂SO₄, filtered and concentrated to deliver an orange oil. This residue was then purified by flash column chromatography (three sequential columns required), eluting with 0-10% MeOH:DCM to afford the free base of the title compound as a clear orange oil (25 mg, 11%).

MS (ES+) m/z 473 (MH⁺)

¹H NMR (400 MHz) δ(CDCl₃) 1.72-1.81 (1H, m), 2.12-2.17 (1H, m), 2.83-3.01 (3H, m), 3.12-3.19 (1H, m), 3.32 (1H, dd,), 3.67 (3H, s), 3.92 (2H, s), 4.27-4.29 (2H, m), 4.32-4.35 (2H, m), 6.74 (1H, d), 6.85 (1H, s), 7.41 (1H, d), 8.07 (1H, d), 8.13 (1H, s), 8.27 (1H, s), 8.37 (1H, d).

The free base of the title compound was converted to the HCl salt by dissolving the free base in MeOH, adding 1 M HCl in MeOH (0.08 ml) and then removing the solvent to give the hydrochloride salt of the title compound (25 mg).

### Example 9

### Racemic 6-{[({6-[6-(Methyloxy)-3-oxopyrido[2,3-b]pyrazin-4(3H)-yl]-1,2,3,4-tetrahydro-1-naphthalenyl}methyl)amino]methyl}-2H-pyrido[3,2-b][1,4]oxazin-3(4H)-one hydrochloride

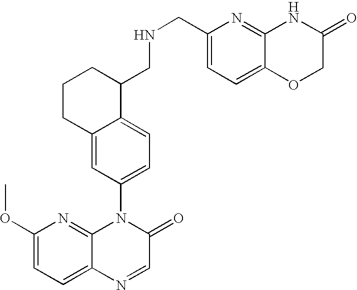

### (a) 6-Amino-1-[(trimethylsilyl)oxy]-1,2,3,4-tetrahydro-1-naphthalenecarbonitrile

Lithium methoxide (0.187 g, 5.0 mmol) was dissolved in THF (100 ml) at room temperature under argon where trimethylsilyl cyanide (15.9 ml, 119 mmol) was added dropwise. The reaction was left stirring at room temperature for 10 mins when 6-amino-3,4-dihydro-1(2H)-naphthalenone (16.0 g, 99.4 mmol) was added portion wise. This mixture was left to stir overnight. Further lithium methoxide (0.187 g, 5.0 mmol) was added and the reaction was left for another couple of hours. The reaction was then diluted with 10% NaHCO₃ (sat. aq.) in H₂O (100 ml) and DCM (200 ml). The aqueous was separated and then extracted a further 3 times with DCM (100 ml). The organics were then combined, dried (Na₂SO₄), filtered and the solvent was removed to give a (8:2) mixture of the title compound and starting material as a black oil (25.6 g, 79% [based on 8:2 mix]).

MS (ES+) m/z 261 (MH⁺).

### (b) 6-Amino-3,4-dihydro-1-naphthalenecarbonitrile

A (8:2) mixture of 6-amino-1-[(trimethylsilyl)oxy]-1,2,3,4-tetrahydro-1-naphthalenecarbonitrile and 6-amino-3,4-dihydro-1(2H)-naphthalenone (15.23 g, 46.8 mmol [based on 8:2 mix]) was dissolved in toluene (300 ml) at room temperature under argon then p-toluenesulphonic acid (0.5 g) was added. The reaction was heated to reflux for 2 h then cooled to room temperature. Solvent was then removed and the residue purified using chromatography eluting with 0-100% EtOAc in hexane then 0-20% MeOH in EtOAc gradient to give a (8:2) mixture of the title compound and 6-amino-3,4-dihydro-1(2H)-naphthalenone as a brown oil (10.3 g).

MS (ES+) m/z 171 (MH⁺).

### (c) 6-Amino-1,2,3,4-tetrahydro-1-naphthalenecarbonitrile

A (8:2) mixture of 6-amino-3,4-dihydro-1-naphthalenecarbonitrile and 6-amino-3,4-dihydro-1(2H)-naphthalenone (8.81 g, 41.4 mmol [based on 8:2 mix]) was dissolved in EtOH (250 ml) at room temperature under argon then sodium borohydride (3.92 g, 104 mmol) was added. The reaction was stirred at room temperature for 10 mins then heated to 40° C. overnight. Reaction was then quenched with H₂O (50 ml) and the diluted with DCM (50 ml). The aqueous was then separated and then washed a further 3 times with DCM. The organics were then combined, dried (Na₂SO₄), filtered and the solvent was removed to give a yellow oil. This residue was then purified using flash column chromatography eluting with 0-100% EtOAc in hexane then 0-20% MeOH in EtOAc gradient to give the title compound as a yellow oil (7.11 g, 100%).

MS (ES+) m/z 173 (MH⁺).

### (d) 1,1-Dimethylethyl[(6-amino-1,2,3,4-tetrahydro-1-naphthalenyl)methyl]-carbamate

6-Amino-1,2,3,4-tetrahydro-1-naphthalenecarbonitrile (7.0 g, 40.7 mmol) was dissolved in ammonium hydroxide (50 ml) and EtOH (230 ml) at room temperature under argon, where Raney nickel (3 spatula's worth) was added. The reaction was then placed under 3.5 bar H₂ and left overnight. The reaction was then filtered (N.B. nickel residues were disposed of separately by carefully quenching with H₂O and 5M HCl) and the solvent removed to give a material whose spectroscopic properties were consistent with 5-(aminomethyl)-5,6,7,8-tetrahydro-2-naphthalenamine. This residue was then dissolved in MeOH (140 ml) at room temperature under argon, cooled to 0° C., then treated with NaHCO₃ (9.70 g, 115 mmol) and di-tent-butyl dicarbonate (9.24 g, 42.3 mmol) were added. After 2 h at 0° C. the reaction was filtered and evaporated to dryness and the residue was then purified using flash column chromatography eluting with 0-100% EtOAc in hexane then 0-20% MeOH in EtOAc to give the title compound as a yellow oil (5.92 g, 56%).

MS (ES+) m/z 299 (MNa⁺).

### (e) 1,1-Dimethylethyl [(6-{[6-(methyloxy)-3-nitro-2-pyridinyl]amino}-1,2,3,4-tetrahydro-1-naphthalenyl)methyl]carbamate

1,1-Dimethylethyl[(6-amino-1,2,3,4-tetrahydro-1-naphthalenyl)methyl]-carbamate (5.92 g, 21.4 mmol) was dissolved in DMF (120 ml) at room temperature under argon then 2-chloro-6-methoxy-3-nitropyridine (4.04 g, 21.4 mmol) and NaHCO₃ (3.60 g, 42.9 mmol) were added. The mixture was then heated to 50° C. and left overnight. The reaction was then cooled to room temperature and diluted with H₂O (150 ml) and DCM (150 ml). The aqueous was then separated and then washed a further 3 times with DCM. The organics were then combined, dried (Na₂SO₄), filtered and the solvent was removed to give a yellow oil. This residue was then purified using flash column chromatography eluting with 0-100% EtOAc in hexane then 0-20% MeOH in EtOAc to give the title compound as a yellow oil (10.8 g).

MS (ES+) m/z 451 (MH⁺).

### (f) 1,1-Dimethylethyl [(6-{[3-amino-6-(methyloxy)-2-pyridinyl]amino}-1,2,3,4-tetrahydro-1-naphthalenyl)methyl]carbamate

1,1-Dimethylethyl [(6-{[6-(methyloxy)-3-nitro-2-pyridinyl]amino}-1,2,3,4-tetrahydro-1-naphthalenyl)methyl]carbamate (10.8 g, 21.4 mmol assuming 100% conversion from previous step) was dissolved in EtOH (400 ml) at room temperature under argon then 10% Pd/C (7 g) was added. Reaction was then placed under an atmosphere of hydrogen at room temperature for 3 h. This mixture was then filtered, the organic layer was collected and the solvent removed to give the title compound as a dark yellow oil (8.54 g, 100%).

MS (ES+) m/z 399 (MH⁺).

### (g) Ethyl N-[2-({5-[({[(1,1-dimethylethyl)oxy]carbonyl}amino)methyl]-5,6,7,8-tetrahydro-2-naphthalenyl}amino)-6-(methyloxy)-3-pyridinyl]glycinate

1,1-Dimethylethyl [(6-{[3-amino-6-(methyloxy)-2-pyridinyl]amino}-1,2,3,4-tetrahydro-1-naphthalenyl)methyl]carbamate (8.54 g, 21.4 mmol, assuming 100% conversion from previous step) was dissolved in DMF (200 mL) and acetonitrile (200 ml) under argon at room temperature, then potassium carbonate (5.92 g, 42.9 mmol) and ethyl bromoacetate (2.38 ml, 21.4 mmol) were then added and the reaction was allowed to stir at room temperature overnight. A further portion of ethyl bromoacetate (0.4 eq.) was added at room temperature and after 1 h the reaction was then heated to 40° C. for a further 5 h. The reaction was cooled and diluted with H₂O (200 ml) and EtOAc (200 ml). The aqueous was then separated and then washed a further 3 times with EtOAc (200 ml). The organics were then combined, dried (Na₂SO₄), filtered and the solvent was removed to give a black oil. This residue was then purified using flash column chromatography eluting with 0-100% EtOAc in hexane then 0-30% MeOH in EtOAc to give the title compound as a black oil (10.3 g, 99%).

MS (ES+) m/z 485 (MH⁺).

(h) 1,1-Dimethylethyl ({6-[6-(methyloxy)-3-oxo-2,3-dihydropyrido[2,3-b]pyrazin-4(1H)-yl]-1,2,3,4-tetrahydro-1-naphthalenyl}methyl)carbamate Ethyl N-[2-({5-[({[(1,1-dimethylethyl)oxy]carbonyl}amino)methyl]-5,6,7,8-tetrahydro-2-naphthalenyl}amino)-6-(methyloxy)-3-pyridinyl]glycinate (10.3 g, 21.2 mmol) was dissolved in THF (400 mL) at room temperature under argon, then it was cooled to 0° C. Potassium tert-butoxide in THF (1M; 5.30 ml, 5.30 mmol) was added dropwise. After 10 mins the reaction was quenched with aqueous ammonium chloride (200 ml) at 0° C. followed by water (200 ml) and EtOAc (200 ml). The aqueous was then separated and washed with EtOAc (3×100 ml). The combined organics were dried (Na₂SO₄), filtered and the solvent was removed to give the title compound as a dark oil (9.00 g, 96%).

MS (ES+) m/z 461 (MNa⁺).

### (i) 1,1-Dimethylethyl ({6-[6-(methyloxy)-3-oxopyrido[2,3-b]pyrazin-4(3H)-yl]-1,2,3,4-tetrahydro-1-naphthalenyl}methyl)carbamate

1,1-Dimethylethyl ({6-[6-(methyloxy)-3-oxo-2,3-dihydropyrido[2,3-b]pyrazin-4(1H)-yl]-1,2,3,4-tetrahydro-1-naphthalenyl}methyl)carbamate (9.00 g, 20.5 mmol) was dissolved in DCM (500 ml) at room temperature under argon then manganese (II) oxide (26.8 g, 308 mmols) was added portionwise. After 1 h, the reaction was filtered, washing further with DCM. The solvent was removed to give a black oil. This residue was then purified using flash column chromatography eluting 0-100% EtOAc in hexane then 0-30% MeOH in EtOAc to give the title compound as a brown oil (5.33 g, 60%).

MS (ES+) m/z 459 (MNa⁺).

### (j) 4-[5-(Aminomethyl)-5,6,7,8-tetrahydro-2-naphthalenyl]-6-(methyloxy)pyrido[2,3-b]pyrazin-3(4H)-one hydrochloride

1,1-Dimethylethyl ({6-[6-(methyloxy)-3-oxopyrido[2,3-b]pyrazin-4(3H)-yl]-1,2,3,4-tetrahydro-1-naphthalenyl}methyl)carbamate (5.33 g, 12.2 mmol) was dissolved in MeOH (15 ml) and CHCl₃ (15 ml) at room temperature under argon where 4M HCl in 1,4-dioxane (15 ml) was added dropwise. After 2 h, the solvent was removed to give title compound as a brown solid (5.41 g, 119%).

MS (ES+) m/z 359 (MH⁺ for the free amine).

### (k) 4-[5-(Aminomethyl)-5,6,7,8-tetrahydro-2-naphthalenyl]-6-(methyloxy)pyrido-[2,3-b]pyrazin-3(4H)-one

4-[5-(Aminomethyl)-5,6,7,8-tetrahydro-2-naphthalenyl]-6-(methyloxy)pyrido[2,3-b]pyrazin-3(4H)-one hydrochloride (4.81 g, 12.9 mmol) was dissolved in 10% MeOH in DCM (100 ml). This organic layer was washed with NaHCO₃ (20 ml), the aqueous was separated, pH was checked to be ˜8, and washed a further 3 times with 10% MeOH in DCM (50 ml). The organics were then combined, dried (Na₂SO₄), filtered and the solvent was removed to give the title compound (3.44 g, 79%).

MS (ES+) m/z 359 (MH⁺).

### (l) Title Compound

4-[5-(Aminomethyl)-5,6,7,8-tetrahydro-2-naphthalenyl]-6-(methyloxy)-pyrido[2,3-b]pyrazin-3(4H)-one hydrochloride (0.2 g, 0.536 mmol) was dissolved in CHCl₃ (5 ml) and MeOH (0.5 ml) at room temperature under argon was treated with triethylamine (0.150 ml, 1.07 mmol), The reaction was stirred for 15 mins when pH was checked (7-8) when 3-oxo-3,4-dihydro-2H-pyrido[3,2-b][1,4]oxazine-6-carbaldehyde (for a synthesis, see WO03087098 Example 31(e)) (0.096 g, 0.536 mmol) was added. Again the mixture was stirred for a further 15 mins before it was treated with sodium triacetoxyborohydride (0.342 g, 1.61 mmol) and then allowed stir at room temperature overnight. The reaction was quenched with saturated aqueous NaHCO₃ solution (10 ml). The aqueous layer was separated and washed a further 3 times with 10% MeOH in DCM (10 ml). The organic layers were then combined, dried (Na₂SO₄), filtered and the solvent removed to give a dark brown oil. This residue was then purified twice using flash column chromatography eluting 0-100% EtOAc in hexane then 0-30% MeOH in EtOAc gradient to give the free base of the title compound as a clear oil (0.157 g, 59%).

MS (ES+) m/z 499 (MH⁺).

¹H NMR (250 MHz) δ(MeOD) 1.71-2.02 (4H, m), 2.79-3.00 (4H, m), 3.10-3.18 (1H, m), 3.58 (3H, s), 3.85 (1H, d), 3.90 (1H, d), 4.61 (2H, s), 6.73 (1H, d), 6.96-7.08 (3H, m), 7.24 (1H, d), 7.38 (1H, d), 8.06 (1H, d) and 8.14 (1H, s).

The free base of the title compound was converted to the HCl salt by dissolving the free base in MeOH (2 ml), adding 1M HCl in MeOH and removing the solvent to give the title compound as a solid (0.151 g).

### Example 10

### 4-(5-{[(2,3-Dihydro[1,4]dioxino[2,3-c]pyridin-7-ylmethyl)amino]-methyl}-5,6,7,8-tetrahydro-2-naphthalenyl)-6-(methyloxy)pyrido[2,3-b]pyrazin-3(4H)-one hydrochloride

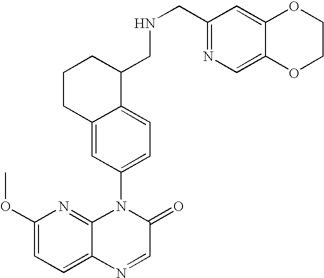

4-[5-(Aminomethyl)-5,6,7,8-tetrahydro-2-naphthalenyl]-6-(methyloxy)pyrido[2,3-b]pyrazin-3(4H)-one hydrochloride (for a preparation see Example 9(k)) (0.2 g, 0.536 mmol) was dissolved in CHCl₃ (5 ml) and MeOH (0.5 ml) at room temperature under argon was treated with triethylamine (0.150 ml, 1.07 mmol), The reaction was stirred for 15 mins then 2,3-dihydro[1,4]dioxino[2,3-c]pyridine-7-carbaldehyde (for synthesis see WO2004058144 Example 2(c) or WO2003087098 Example 19(d)) (0.089 g, 0.536 mmol) was added. The mixture was stirred for a further 15 mins before it was treated with sodium triacetoxyborohydride (0.342 g, 1.61 mmol) and then allowed stir at room temperature overnight. The reaction was quenched with saturated aqueous NaHCO₃ solution (10 ml). The aqueous layer was separated and washed a further 3 times with 10% MeOH in DCM (10 ml). The organic layers were then combined, dried (Na₂SO₄), filtered and the solvent removed to give a dark brown oil. This residue was then purified twice using flash column chromatography eluting 0-100% EtOAc in hexane then 0-30% MeOH in EtOAc gradient to give the free base of the title compound as a clear oil (0.104 g, 40%).

MS (ES+) m/z 486 (MH⁺).

¹H NMR (250 MHz) δ(MeOD) 1.72-2.02 (4H, m), 2.78-2.95 (4H, m), 3.06-3.13 (1H, m), 3.58 (3H, s), 3.80 (1H, d), 3.85 (1H, d), 4.26-4.36 (4H, m), 6.74 (1H, d), 6.96-7.08 (3H, m), 7.37 (1H, d), 8.00 (1H, s), 8.75 (1H, d) and 8.13 (1H, s).

The free base of the title compound was converted to the HCl salt by dissolving the obtained free base in MeOH (2 ml), adding 1M HCl in MeOH and removing the solvent to give a solid (0.075 g)

### Example 11

### 4-(5-{[(6,7-Dihydro[1,4]dioxino[2,3-c]pyridazin-3-ylmethyl)amino]-methyl}-5,6,7,8-tetrahydro-2-naphthalenyl)-6-(methyloxy)pyrido[2,3-b]pyrazin-3(4H)-one hydrochloride

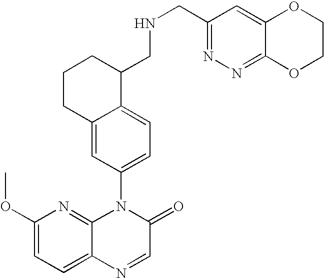

4-[5-(Aminomethyl)-5,6,7,8-tetrahydro-2-naphthalenyl]-6-(methyloxy)pyrido[2,3-b]pyrazin-3(4H)-one hydrochloride (for a preparation see Example 9(k)) (0.2 g, 0.536 mmol) was dissolved in CHCl₃ (5 ml) and MeOH (0.5 ml) at room temperature under argon was treated with triethylamine (0.150 ml, 1.07 mmol), The reaction was stirred for 15 mins when pH was checked (7-8) when 6,7-dihydro[1,4]dioxino[2,3-c]pyridazine-3-carbaldehyde (for a preparation see Example 5(1)) (0.089 g, 0.536 mmol) was added. Again the mixture was stirred for a further 15 mins before it was treated with sodium triacetoxyborohydride (0.342 g, 1.61 mmol) and then allowed stir at room temperature overnight. A further 3 equivalents of sodium triacetoxyborohydride (0.342 g, 1.61 mmol) was then added and after 2 h the reaction was quenched with saturated aqueous NaHCO₃ solution (10 ml). The aqueous layer was separated and washed a further 3 times with 10% MeOH in DCM (10 ml). The organic layers were then combined, dried (Na₂SO₄), filtered and the solvent removed to give a dark brown oil. This residue was then purified twice using flash column chromatography eluting 0-100% EtOAc in hexane then 0-30% MeOH in EtOAc gradient to give the free base of the title compound as a clear oil (0.153 g, 59%).

MS (ES+) m/z 487 (MH⁺).

¹H NMR (250 MHz) δ(MeOD) 1.72-2.02 (4H, m), 2.69-3.03 (4H, m), 3.10-3.17 (1H, m), 3.57 (3H, s), 4.02-4.13 (2H, m), 4.40-4.45 (2H, m), 4.51-4.56 (2H, m), 6.73 (1H, d), 7.04-7.08 (2H, m), 7.24 (1H, s), 7.40 (1H, d), 8.05 (1H, d) and 8.11 (1H, s).

The free base of the title compound material was converted to the HCl salt by dissolving the obtained free base in MeOH (2 ml), adding 1M HCl in MeOH and removing the solvent to give a solid (0.147 g).

### Example 12

### Cis-6-[({6-Hydroxy-3-[6-(methyloxy)-3-oxopyrido[2,3-b]pyrazin-4(3H)-yl]-5,6,7,8-tetrahydro-7-quinolinyl}amino)methyl]-2H-pyrido[3,2-b][1,4]oxazin-3(4H)-one dihydrochloride

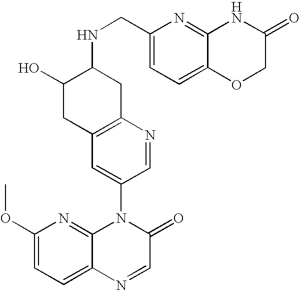

### (a) 4-Hydroxy-2-cyclohexen-1-one

This method was adapted from the procedure of Danishefsky, S. J.; Simoneau, B. J. Am. Chem. Soc. 1989, 111, 2599-2604 and references cited therein.

To a solution of 1-methoxy-cyclohexa-1,4-diene (10 mL, 131 mmol) in MeOH:H₂O (3:1, 80 mL), was added oxalic acid (385 mg) in one portion. The resulting solution was stirred for 30 min then extracted with DCM (×3). The combined organics were dried over Na₂SO₄, filtered and concentrated to deliver 3-cyclohexen-1-oneas an oil. To a solution of this material in DCM (150 mL) at room temperature was added a suspension of meta-chloroperbenzoic acid (33.1 g, 96.03 mmol, 50% purity) in DCM (150 mL) dropwise over 10 min. The resulting solution was stirred for 18 h, then filtered. The filtrate was washed with 10% aq. Na₂S₂O₃, sat. aq. NaHCO₃, H₂O, brine (150 mL) and the organic layer was dried over Na₂SO₄, filtered and concentrated to afford the crude epoxide. To a solution of the residue in DCM:diethyl ether (1:1, 200 mL) was added basic alumina (34.5 g, activity 1) in one portion. The resulting suspension was stirred vigorously for 1 h then filtered, washing the solid thoroughly with DCM (˜1 L). The filtrate was concentrated to deliver the crude product as a clear yellow oil. The residue was purified by flash column chromatography (eluting with petrol:EtOAc 2:1-1:2) to deliver a clear, colourless oil (4.54 g, 46% over 3 steps).

MS (ES+) m/z 113 (MH⁺).

### (b) Cis-1,1-Dimethylethyl (2-hydroxy-5-oxocyclohexyl)carbamate

To a solution of 4-hydroxy-2-cyclohexen-1-one (1.13 g, 10.1 mmol) and tert-butyl carbamate (1.18 g, 10.1 mmol) in DCM (3.5 mL) at room temperature, was added Bi(NO₃)₃ (489 mg, 1.01 mmol) in one portion. The resulting suspension was stirred for 18 h then diluted with DCM and washed with sat. aq. NaHCO₃. The organic layer was dried over MgSO₄, filtered and concentrated to afford a yellow gum. The residue was purified by flash column chromatography, eluting with petrol:EtOAc 3:2-1:1 to afford a white solid (842 mg, 36%).

MS (ES+) m/z 252 (MNa⁺).

### (c) Cis-1,1-Dimethylethyl (6-hydroxy-3-nitro-5,6,7,8-tetrahydro-7-quinolinyl)carbamate

A solution of 1-methyl-3,5-dinitro-2(1H)-pyridinone (for a synthesis see Demartino, J. et al, US 2006030582, Intermediate [0328]) (732 mg, 3.7 mmol) and cis-1,1-dimethylethyl (2-hydroxy-5-oxocyclohexyl)carbamate (839 mg, 3.7 mmol) in 1 M NH₃/MeOH (20 mL) was heated at 65° C. for 1.5 h then cooled to room temperature. The reaction mixture was then concentrated then partitioned between DCM and H₂O. The organic layer was separated, dried over MgSO₄, filtered and evaporated to deliver an orange solid. The residue was purified by flash column chromatography, eluting with petrol:EtOAc 1:1-1:2 to afford a white solid (377 mg, 33%).

¹H NMR (400 MHz) δ(CDCl₃) 1.47 (9H, s), 2.87 (1H, s), 3.07 (1H, dd), 3.13-3.23 (2H, m), (1H, dd), 3.31 (1H, dd), 4.07-4.15 (1H, m), 4.38-4.41 (1H, m), 4.91 (1H, m), 8.23 (1H, d), 9.22 (1H, d).

### (d) Cis-1,1-Dimethylethyl (3-amino-6-hydroxy-5,6,7,8-tetrahydro-7-quinolinyl)carbamate

A solution of cis-1,1-dimethylethyl (6-hydroxy-3-nitro-5,6,7,8-tetrahydro-7-quinolinyl)carbamate (4.25 g) in ethanol (200 mL) at room temperature, was purged with argon then 10% palladium on charcoal paste (0.5 g) was added in one portion. The resulting suspension was evacuated and purged with argon three times, then subjected to an atmosphere of hydrogen and stirred vigorously at room temperature for 16 h. The reaction mixture was evacuated and purged with argon several times then the suspension was filtered through a pad of Celite and the precipitate was washed extensively with ethanol. The filtrate was concentrated to deliver a brown oil. The residue was purified by flash column chromatography, eluting with 0-10% MeOH:DCM (with 1 drop concentrated ammonia solution/100 mL eluant) to afford a brown solid (2.16 g)

MS (ES+) m/z 280 (MH⁺).

### (e) Cis-1,1-Dimethylethyl (6-hydroxy-3-{[5-(methyloxy)-2-nitrophenyl]amino}-5,6,7,8-tetrahydro-7-quinolinyl)carbamate

To a solution of cis-1,1-dimethylethyl (3-amino-6-hydroxy-5,6,7,8-tetrahydro-7-quinolinyl)carbamate (200 mg, 0.72 mmol) in DMF (3 mL) at room temperature under argon was added 2-chloro-6-(methyloxy)-3-nitropyridine (135 mg, 0.72 mmol) then sodium hydrogen carbonate (121 mg, 1.44 mmol). The resulting reaction mixture was heated at 60° C. overnight then cooled to room temperature and concentrated. The residue was partitioned between DCM and H₂O and separated. The aqueous layer was extracted several times with DCM and the combined organic extracts were washed with brine, dried over MgSO₄, filtered and concentrated to deliver a yellow oil.

Separately, to a solution of cis-1,1-dimethylethyl (3-amino-6-hydroxy-5,6,7,8-tetrahydro-7-quinolinyl)carbamate (2.17 g, 7.77 mmol) in DMF (32 mL) at room temperature under argon was added 2-chloro-6-(methyloxy)-3-nitropyridine (1.47 g, 7.77 mmol) then sodium hydrogen carbonate (1.31 g, 15.54 mmol). The resulting reaction mixture was heated at 60° C. overnight then cooled to room temperature and concentrated. The residue was partitioned between DCM and H₂O and separated. The aqueous layer was extracted several times with DCM and the combined organic extracts were washed with brine, dried over MgSO₄, filtered and concentrated to deliver a brown oil.

These combined crude oils were combined and subjected to flash column chromatography, eluting with 0-2% MeOH:DCM to afford a yellow solid (2.46 g, 67%).

MS (ES+) m/z 432 (MH⁺).

### (f) Cis-Ethyl N-[2-{[7-({[(1,1-dimethylethyl)oxy]carbonyl}amino)-6-hydroxy-5,6,7,8-tetrahydro-3-quinolinyl]amino}-6-(methyloxy)-3-pyridinyl]glycinate

To a solution of cis-1,1-dimethylethyl (6-hydroxy-3-{[5-(methyloxy)-2-nitrophenyl]amino}-5,6,7,8-tetrahydro-7-quinolinyl)carbamate (2.46 g, 5.71 mmol) in ethanol (100 mL) at room temperature, was purged with argon then 10% palladium on charcoal paste (1 g) was added in one portion. The resulting suspension was evacuated and purged with argon three times, then subjected to an atmosphere of hydrogen and stirred vigorously at room temperature for 2 h. The reaction mixture was evacuated and purged with argon several times. The suspension was filtered through a pad of keiselguhr and the precipitate was washed extensively with ethanol. The filtrate was concentrated to deliver a dark green oil which was used in the next step without further purification.

To a solution of this material in toluene (60 mL) at room temperature was added ethyl glyoxylate (1.36 mL, 6.85 mmol, 50% solution in toluene) and the resulting dark green solution was stirred at room temperature for 1 h. Methanol (60 mL) was added to the reaction mixture and the resulting solution was cooled to 0° C. Sodium borohydride (238 mg, 6.28 mmol) was added to the reaction mixture in one portion and vigorous gas evolution was observed. The mixture was then warmed to room temperature and stirred for 10 min. The reaction was quenched with H₂O and the reaction mixture was concentrated to half the original volume. The layers were separated and the aqueous layer was extracted several times with ethyl acetate and the combined organic extracts were dried over MgSO₄, filtered and concentrated to deliver a green foam. The residue was purified by flash column chromatography, eluting with 0-5% MeOH:DCM to afford a yellow foam (2.67 g, 60%).

MS (ES+) m/z 488 (MH⁺).

### (g) Cis-Ethyl N-[2-{[6-{[(1,1-dimethylethyl)(dimethyl)silyl]oxy}-7-({[(1,1-dimethylethyl)oxy]carbonyl}amino)-5,6,7,8-tetrahydro-3-quinolinyl]amino}-6-(methyloxy)-3-pyridinyl]glycinate

To a solution of cis-ethyl N-[2-{[7-({[(1,1-dimethylethyl)oxy]carbonyl}amino)-6-hydroxy-5,6,7,8-tetrahydro-3-quinolinyl]amino}-6-(methyloxy)-3-pyridinyl]glycinate (1.35 g, 2.77 mmol) in DMF (4.5 mL) at room temperature, was added tert-butyldimethylchlorosilane (418 mg, 2.77 mmol) then imidazole (207 mg, 3.05 mmol). The resulting solution was stirred for 18 h then further tert-butyldimethylchlorosilane (84 mg, 0.55 mmol) and imidazole (38 mg, 0.55 mmol) were added. After a further 2 h the reaction mixture was diluted with H₂O (40 mL) and extracted with DCM (3×40 mL). The combined organic layers were washed with H₂O (40 mL), brine (80 mL) then dried over MgSO₄, filtered and concentrated to deliver a clear, orange oil. The residue was purified by flash column chromatography, eluting with petrol:EtOAc 2:1-1:1 to afford a clear, orange oil (977 mg, 59%).

MS (ES+) m/z 602 (MH⁺).

### (h) Cis-1,1-Dimethylethyl {6-hydroxy-3-[6-(methyloxy)-3-oxopyrido[2,3-b]pyrazin-4(3H)-yl]-5,6,7,8-tetrahydro-7-quinolinyl}carbamate

To a suspension of sodium hydride (86 mg, 2.15 mmol, 60% dispersion in mineral oil) in THF (50 mL) at 0° C., was added via cannula a solution of cis-ethyl N-[2-{[6-{[(1,1-dimethylethyl)(dimethyl)silyl]oxy}-7-({[(1,1-dimethylethyl)oxy]carbonyl}amino)-5,6,7,8-tetrahydro-3-quinolinyl]amino}-6-(methyloxy)-3-pyridinyl]glycinate (1.17 g, 1.95 mmol) in THF (50 mL) dropwise over 15 min. The resulting solution was stirred at 0° C. for 20 min then the reaction was quenched with the minimum volume of sat. aq. NH₄Cl (˜1 mL). The reaction was warmed to room temperature, diluted with EtOAc and an excess of Na₂SO₄ was added. The resulting suspension was stirred vigorously at room temperature for 5 min then filtered and concentrated to deliver an orange powder, which was used in the next step without further purification.

To a solution of this material in DCM (100 mL) at room temperature was added MnO₂ (2.54 g, 29.25 mmol) in one portion. The resulting suspension was stirred at room temperature for 1 h, filtered and the filtrate concentrated to deliver a brown foam, which was used in the next step without further purification.

To a solution of this material in THF (5 mL) at 0° C., was added dropwise tetrabutyalammoniumfluoride (2.15 mL, 2.15 mmol, 1 M solution in THF). The resulting solution was stirred at 0° C. for 20 min then warmed to room temperature. After a further 45 min at room temperature the reaction was quenched with water (50 mL) and stirred for 10 min. The reaction mixture was extracted with EtOAc (3×50 mL) and the combined organic layers were washed with brine, dried over MgSO₄, filtered and concentrated to deliver a brown oil. The residue was purified by flash column chromatography, eluting with 0-5% MeOH:DCM to afford a light brown powder (776 mg, 91% over 3 steps).

MS (ES+) m/z 440 (MH⁺).

### (i) Cis-4-(7-Amino-6-hydroxy-5,6,7,8-tetrahydro-3-quinolinyl)-6-(methyloxy)pyrido[2,3-b]pyrazin-3(4H)-one

To a solution of cis-1,1-dimethylethyl {6-hydroxy-3-[6-(methyloxy)-3-oxopyrido[2,3-b]pyrazin-4(3H)-yl]-5,6,7,8-tetrahydro-7-quinolinyl}carbamate (764 mg, 1.74 mmol) in DCM (10 mL) at room temperature, was added dropwise 4 M HCl in 1,4-dioxane (1 mL). After 1 h at room temperature another 1 mL of acid was added and stirring was continued for a further 2 h at room temperature. Another 2 mL of acid was added and stirring was continued for a further 30 min then the reaction mixture was concentrated to deliver an orange solid. The residue was purified twice using an SCX cartridge, eluting with 0-100% MeOH:DCM then 0-100% 2 M NH₃ in MeOH:MeOH to afford a pale orange solid (553 mg, 94%).

MS (ES+) m/z 340 (MH⁺).

### (j) Title Compound

To a solution of cis-4-(7-amino-6-hydroxy-5,6,7,8-tetrahydro-3-quinolinyl)-6-(methyloxy)pyrido[2,3-b]pyrazin-3(4H)-one (131 mg, 0.39 mmol) in DCM (5 mL) and MeOH (0.5 mL) at room temperature was added 3-oxo-3,4-dihydro-2H-pyrido[3,2-b][1,4]oxazine-6-carbaldehyde (for a synthesis see WO03087098 Example 31(e)) (104 mg, 0.43 mmol) then sodium triacetoxyborohydride (165 mg, 0.78 mmol). The resulting suspension was stirred at room temperature overnight then the reaction mixture was diluted with DCM (10 mL) and washed with sat. aq. NaHCO₃ (10 mL). The aqueous layer was then separated and washed a further three times with DCM (10 mL). The combined organic layers were then dried over Na₂SO₄, filtered and concentrated to deliver a yellow oil. This residue was then purified by flash column chromatography, eluting with 0-10% MeOH:DCM to afford the free base of the title compound as a yellow oil (94 mg, 48%).

MS (ES+) m/z 502 (MH⁺).

¹H NMR (400 MHz) δ(CDCl₃) 3.08-3.11 (2H, m), 3.14-3.22 (3H, m), 3.66 (3H, s), 3.99 (2H, s), 4.29-4.30 (2H, s), 4.64 (2H, s), 6.74 (1H, d), 6.92 (1H, d), 7.22 (1H, d), 7.46 (1H, d), 8.07 (1H, d), 8.26 (1H, s), 8.39 (1H, d).

The free base of the title compound was converted to the title compound by dissolving in MeOH, adding a large excess of 1 M HCl in MeOH (˜2 mL) and then removing the solvents to give a solid (84 mg).

### Example 13

### Cis-4-{7-[(6,7-Dihydro[1,4]dioxino[2,3-c]pyridazin-3-ylmethyl)amino]-6-hydroxy-5,6,7,8-tetrahydro-3-quinolinyl}-6-(methyloxy)pyrido[2,3-b]pyrazin-3(4H)-one dihydrochloride

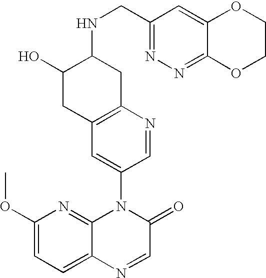

To a solution of cis-4-(7-amino-6-hydroxy-5,6,7,8-tetrahydro-3-quinolinyl)-6-(methyloxy)pyrido[2,3-b]pyrazin-3(4H)-one (for a preparation see Example 12(i)) (131 mg, 0.39 mmol) in DCM (5 mL) and MeOH (0.5 mL) at room temperature was added 6,7-dihydro[1,4]dioxino[2,3-c]pyridazine-3-carbaldehyde (for a preparation see Example 5(1)) (85 mg, 0.51 mmol) then sodium triacetoxyborohydride (248 mg, 1.17 mmol). The resulting suspension was stirred at room temperature overnight then the reaction mixture was diluted with DCM (10 mL) and washed with sat. aq. NaHCO₃ (10 mL). The aqueous was then separated and washed a further three times with DCM (10 mL). The combined organic layers were then dried over Na₂SO₄, filtered and concentrated to deliver a yellow foam. This residue was then purified thrice by flash column chromatography, eluting with 0-10% MeOH:DCM to afford the free base of the title compound a yellow oil (28 mg, 15%).

MS (ES+) m/z 490 (MH⁺).

¹H NMR (400 MHz) δ(CDCl₃) 3.07-3.14 (4H, m), 3.21-3.25 (1H, m), 3.67 (3H, s), 4.14 (2H, dd), 4.24-4.27 (1H, m), 4.38-4.40 (2H, m), 4.52-4.54 (2H, m), 6.74 (1H, d), 6.92 (1H, d), 7.00 (1H, s), 7.45 (1H, d), 8.07 (1H, d), 8.26 (1H, s), 8.39 (1H, d).

The free base of the title compound was converted to the HCl salt by dissolving in MeOH, adding a large excess of 1 M HCl in MeOH (˜2 mL) and then removing the solvent to give the dihydrochloride salt of the title compound (24 mg).

### Example 14

### Cis-4-{7-[(2,3-Dihydro[1,4]dioxino[2,3-c]pyridin-7-ylmethyl)amino]-6-hydroxy-5,6,7,8-tetrahydro-3-quinolinyl}-6-(methyloxy)pyrido[2,3-b]pyrazin-3(4H)-one

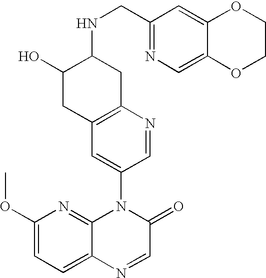

To a solution of cis-4-(7-amino-6-hydroxy-5,6,7,8-tetrahydro-3-quinolinyl)-6-(methyloxy)pyrido[2,3-b]pyrazin-3(4H)-one (for a preparation see Example 12(i)) (131 mg, 0.39 mmol) in DCM (5 mL) and MeOH (0.5 mL) at room temperature was added 2,3-dihydro[1,4]dioxino[2,3-c]pyridine-7-carbaldehyde (for a synthesis see WO2004058144, Example 2(c) or WO2003087098 Example 19(d)) (71 mg, 0.43 mmol) then sodium triacetoxyborohydride (165 mg, 0.78 mmol). The resulting suspension was stirred at room temperature overnight then the reaction mixture was diluted with DCM (10 mL) and washed with sat. aq. NaHCO₃ (10 mL). The aqueous was then separated and washed a further three times with DCM (10 mL). The combined organic layers were then dried over Na₂SO₄, filtered and concentrated to deliver a yellow foam. This residue was then purified twice by flash column chromatography, eluting with 0-10% MeOH:DCM to afford a yellow solid (112 mg, 59%).

MS (ES+) m/z 489 (MH⁺).

¹H NMR (400 MHz) δ(CDCl₃) 3.07-3.13 (4H, m), 3.16-3.19 (1H, m), 3.67 (3H, s), 3.96 (2H, dd), 4.20-4.22 (1H, m), 4.28-4.31 (2H, m), 4.34-4.36 (2H, m), 6.74 (1H, d), 6.79 (1H, s), 7.45 (1H, d), 8.07 (1H, d), 8.13 (1H, s), 8.26 (1H, s), 8.39 (1H, d).

The free base of the title compound was converted to the HCl salt by dissolving in MeOH, adding a large excess of 1 M HCl in MeOH (˜2 mL) and then removing the solvent to give the hydrochloride salt of the title compound (106 mg).

### Example 15

### 4-{7-[(2,3-dihydro[1,4]dioxino[2,3-c]pyridin-7-ylmethyl)amino]-5-methyl-6-oxo-5,6,7,8-tetrahydro-1,5-naphthyridin-3-yl}-6-(methyloxy)pyrido[2,3-b]pyrazin-3(4H)-one

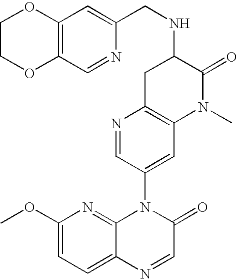

### (a) 2,5-Dibromo-3-nitropyridine

5-Bromo-3-nitro-2(1H)-pyridinone (5 g, 22.83 mmol) was suspended in toluene (50 ml). DMF (0.177 ml, 2.283 mmol) was added and the mixture was protected from light. A solution of phosphorous oxybromide (7.85 g, 27.4 mmol) in toluene (50 ml) was added to the pyridine mixture over 1.5 h at 90° C. The reaction mixture was heated at 90° C. for 14 h, cooled down and extracted with water. The organic layer was washed with 1N NaOH and brine and dried over MgSO₄. The solvent was removed to give title compound (6.45 g, 22.88 mmol, 100% yield) as yellow solid.

MS (ES+) m/z 282 (MH⁺).

### (b) 2,5-Dibromo-3-aminopyridine

2,5-Dibromo-3-nitropyridine (2.5 g, 8.87 mmol) was stirred in acetic acid (10 mL). Powdered iron (2.476 g, 44.3 mmol) was added and the solution heated at 80° C. for 15 minutes with vigorous stirring. The reaction mixture was filtered through Celite washed with ether and EtOAc. The resulting organic layer was washed with water and brine, dried over magnesium sulfate and filtered. The solvent was removed to give the crude product. Crude product was purified on silica eluting with 0-30% EtOAc:hexane to give the title compound (1.7 g, 6.75 mmol, 76% yield) as white solid.

MS (ES+) m/z 252 (MH⁺).

### (c) 1,1-Dimethylethyl (7-bromo-2-oxo-1,2,3,4-tetrahydro-1,5-naphthyridin-3-yl)carbamate

Zinc powder (0.934 g, 14.28 mmol) and iodine (0.054 g, 0.214 mmol) were heated in an evacuated flask which was then flushed with nitrogen 3 times. Methyl N-{[(1,1-dimethylethyl)oxy]carbonyl}-3-iodo-D-alaninate (2.35 g, 7.14 mmol, Aldrich Chemicals) was dissolved in dry DMF (11.74 mL) and transferred via syringe to the reaction mixture which was previously cooled to 0° C. (reaction complete after 1.5 h). The ice bath was removed and 2,5-dibromo-3-pyridinamine (2.392 g, 9.50 mmol) was added followed by bis(triphenylphosphine)palladium(II) chloride (0.251 g, 0.357 mmol) and the mixture heated at 40° C. for 14 h. The mixture was cooled down and filtered through Celite, washing with EtOAc. Solvent was removed in vacuum. The mixture was redissolved in DMF (10 mL) and potassium carbonate (1.283 g, 9.28 mmol) was added. The resulting mixture was stirred at 80° C. for 6 h. The mixture was concentrated and diluted with EtOAc, washed with water and brine and dried over Mg₂SO₄. Solvent was removed and the mixture chromatographed on silica eluting with 0-100% EtOAc:hexane to give the title compound (1.8 g, 5.26 mmol, 73.7% yield) as light yellow solid.

MS (ES+) m/z 343 (MH⁺).

### (d) 1,1-Dimethylethyl (7-bromo-1-methyl-2-oxo-1,2,3,4-tetrahydro-1,5-naphthyridin-3-yl)carbamate

Sodium hydride (0.250 g, 6.24 mmol) and 1,1-dimethylethyl (7-bromo-2-oxo-1,2,3,4-tetrahydro-1,5-naphthyridin-3-yl)carbamate (1.78 g, 5.20 mmol) in THF (35 mL) were mixed at 0° C. Iodomethane (0.342 ml, 5.46 mmol) was added and mixture stirred at 0° C. to room temperature for 18 h. The mixture was quenched with ammonium chloride, diluted with EtOAc, washed with brine and dried over magnesium sulfate. The product was chromatographed on silica eluting with 0-80% EtOAc:hexane to give the title compound (750 mg, 2.105 mmol, 40.5% yield) as white foam.

MS (ES+) m/z 357 (MH⁺).

### (e) [7-({[(1,1-Dimethylethyl)oxy]carbonyl}amino)-5-methyl-6-oxo-5,6,7,8-tetrahydro-1,5-naphthyridin-3-yl]boronic acid

To a −78° C. cooled solution of N-butyllithium (0.225 ml, 0.561 mmol, 2.5M solution in THF) in 1 mL THF was added 1,1-dimethylethyl (7-bromo-1-methyl-2-oxo-1,2,3,4-tetrahydro-1,5-naphthyridin-3-yl)-carbamate (100 mg, 0.281 mmol) in THF (5 ml). The mixture was stirred at −78° C. for 1 h. Trimethyl borate (0.038 ml, 0.337 mmol) was added and the mixture stirred at −78° C. to room temperature for 3 h. The mixture was quenched with water. Solvent was removed in vacuum. The mixture was extracted with EtOAc, washed with 1N HCl, water and brine and dried over magnesium sulfate. Solvent was removed in vacuum to give the title compound (90 mg, 0.281 mmol, 100% yield).

MS (ES+) m/z 322 (MH⁺).

### (f) Title Compound

The title compound is prepared from 1,1-dimethylethyl (7-bromo-1-methyl-2-oxo-1,2,3,4-tetrahydro-1,5-naphthyridin-3-yl)carbamate or [7-({[(1,1-dimethylethyl)oxy]carbonyl}amino)-5-methyl-6-oxo-5,6,7,8-tetrahydro-1,5-naphthyridin-3-yl]boronic acid by procedures generally described herein.

### Example 16

### 4-(8-{[(2,3-Dihydro[1,4]dioxino[2,3-c]pyridin-7-ylmethyl)amino]methyl}-7,8-dihydro-5H-pyrano[4,3-b]pyridin-3-yl)-6-(methyloxy)pyrido[2,3-b]pyrazin-3(4H)-one dihydrochloride

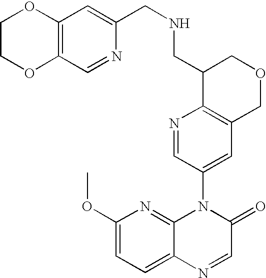

4-[8-(Aminomethyl)-7,8-dihydro-5H-pyrano[4,3-b]pyridin-3-yl]-6-(methyloxy)pyrido[2,3-b]pyrazin-3(4H)-one (for a preparation see Example 30(h)) (0.05 g, 0.147 mmol) and 2,3-dihydro[1,4]dioxino[2,3-c]pyridine-7-carbaldehyde (for synthesis see WO2004058144 Example 2(c) or WO2003087098 Example 19(d)) (0.024 g, 0.147 mmol) was dissolved in chloroform (2 ml) and methanol (0.2 ml) at room temperature under argon, whereupon sodium triacetoxyborohydride (0.094 g, 0.442 mmol) was added portionwise. After 2 hours, the reaction was quenched with saturated NaHCO₃ (5 ml) and diluted with 10% MeOH in DCM (5 ml). The aqueous phase was separated and washed a further 3 times with 10% MeOH in DCM (5 ml). The organics were combined, dried (Na₂SO₄), filtered and the solvent removed to give a yellow oil (0.053 g). This residue was then chromatographed eluting with 0-30% MeOH in DCM, to give the free base of the title compound as a clear oil (0.035 g, 49%).

MS (ES+) m/z 489 (MH⁺).

¹H NMR (400 MHz) δ(MeOD) 2.96-3.08 (2H, m), 3.11-3.17 (1H, m), 3.62 (3H, s), 3.63 (3H, s), 3.85 (2H, d), 4.07 (1H, dd), 4.18 (1H, dd), 4.27-4.30 (2H, m), 4.33-4.37 (2H, m), 4.83 (2H, d), 6.81 (1H, d), 6.89 (1H, s), 7.62 (1H, d), 7.99 (1H, s), 8.13 (1H, d), 8.19 (1H, s), and 8.46 (1H, d).

This compound was converted to the title compound by dissolving the obtained free base in MeOH (2 ml) whereupon (0.145 ml, 2 eq.) of 1M HCl in MeOH was added. This solution was then evaporated to dryness to give the title compound (0.0342 g).

### Example 17

### 4-{3-[(2,3-Dihydro[1,4]dioxino[2,3-c]pyridin-7-ylmethyl)amino]-3,4-dihydro-2H-pyrano[3,2-b]pyridin-7-yl}-6-(methyloxy)pyrido[2,3-b]pyrazin-3(4H)-one fumarate

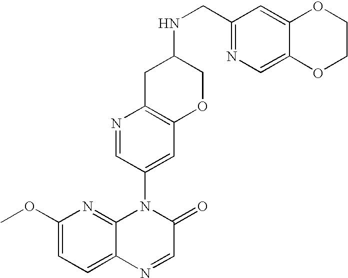

### (a) 5-Bromo-2-iodo-3-pyridinol

5-Bromo-3-pyridinol (25.1 g, 144 mmol) was suspended in water (500 ml), sodium carbonate (45.9 g, 433 mmol) was added and the mixture was stirred until a clear solution formed. Iodine (36.6 g, 144 mmol) was added in portions over 2.75 h while stirring at room temp. Iodine dissolved more quickly as the reaction progressed, giving a dark brown solution which gradually turned to pale yellow. The mixture was stirred for another 30 min, and then added gradually to 150 ml 2M hydrochloric acid. A white precipitate formed. More acid was added as required to bring the final pH to approximately 7. The solid was filtered off, washed with water and dried (50° C., vacuum) to give the product (43.16 g, 100%).

MS (+ve ion electrospray) m/z 300/302 (MH+).

### (b) 5-Bromo-2-[(1Z)-1-propen-1-yl]-3-pyridinol

A mixture of 5-bromo-2-iodo-3-pyridinol (11.16 g, 37.2 mmol), cis-propenyl boronic acid (3.84 g, 44.7 mmol) and potassium carbonate (3.84 g, 149 mmol) in 1,4-dioxane/water (3:1, 320 ml) was degassed by bubbling nitrogen through before addition of tetrakis(triphenylphoshine)palladium(0) (0.860 g, 0.74 mmol). The mixture was heated at 100° C. overnight, and then evaporated. The aqueous residue was filtered under suction, washed through with DMF, and the filtrate was evaporated to approx. half volume. The residue was then used crude in the next step (assumed 100% yield).

MS (+ve ion electrospray) m/z 214/216 (MH+).

### (c) 5-Bromo-2-[(1Z)-1-propen-1-yl]-3-(2-propen-1-yloxy)pyridine

Crude 5-bromo-2-[(1Z)-1-propen-1-yl]-3-pyridinol (assumed 37.2 mmol) in DMF (340 ml) was treated with potassium carbonate (10.28 g, 74.4 mmol) and allyl iodide (3.41 ml, 37.2 ml). The mixture was stirred at room temperature overnight, and then evaporated. The residue was slurried with toluene, filtered under suction and the filtrate was evaporated to give the product (8.9 g, 94% crude), which was used crude in the next step.

MS (+ve ion electrospray) m/z 254/256 (MH+).

### (d) 7-Bromo-2H-pyrano[3,2-b]pyridine

Crude 5-bromo-2-[(1Z)-1-propen-1-yl]-3-(2-propen-1-yloxy)pyridine (8.9 g, 35 mmol) in toluene (350 ml) was degassed by bubbling nitrogen through the solution before addition of benzylidene[1,3-bis(2,4,6-trimethylphenyl)-2-imidazolidinylidene]dichloro(tricyclohexylphospine)ruthenium (Grubbs catalyst, 2^{nd} generation, 0.65 g, 0.766 mmol). The mixture was heated at 90° C. overnight (24 h). Another portion of Grubbs catalyst (0.65 g) was added and heating continued for 24 h. The mixture was evaporated and the crude product was chromatographed on 330 g silica eluted with 0-20% ethyl acetate/cyclohexane to give the product (3.25 g, 44%:41% over 3 steps from 5-bromo-2-iodo-3-pyridinol).

MS (+ve ion electrospray) m/z 212/214 (MH+).

### (e) 2H-Pyrano[3,2-b]pyridin-7-ylboronic acid

A 500 L round bottomed flask was purged with nitrogen, and to it was added dry diethyl ether (50 ml) and n-butyllithium (2.5M in hexanes, 7.66 ml). The solution was cooled to −78° C. 7-Bromo-2H-pyrano[3,2-b]pyridine (3.25 g, 15.33 mmol) was dissolved in diethyl ether (100 ml) in a 250 mL round-bottomed flask to give a brown solution with some undissolved material. This solution was added slowly via cannula to the solution of n-butyllithium in diethyl ether. The mixture was stirred for 5 mins. Triisopropyl borate (4.45 ml, 19.16 mmol) was dissolved in ether (50 ml) in a 100 mL round-bottomed flask to give a colourless solution. The solution was added dropwise to the reaction mixture, which was then removed from the cooling bath and allowed to warm to room temperature. The reaction was quenched with saturated ammonium chloride solution (125 ml), then ethyl acetate (125 ml) and water (50 ml) were added and the phases were separated. The aqueous phase was extracted thoroughly with ethyl acetate/methanol (10-20% methanol), with addition of 5M hydrochloric acid to bring the pH of the aqueous to approximately 5. The organic fractions were combined, dried and evaporated to give the product (2.73 g, 100%) as a yellow solid. This was used in the next step without purification.

MS (+ve ion electrospray) m/z 178 (MH+).

### (f) 6-(methyloxy)pyrido[2,3-b]pyrazin-3(4H)-one

A solution of 6-(methyloxy)-1,4-dihydropyrido[2,3-b]pyrazin-3(2H)-one (for a preparation see Example 26(d)) (10.9 g, 61 mmol) in DMF (200 ml) was treated with MnO₂ (26.4 g). After 2 hours at room temperature no reaction had occurred so more MnO₂ (18 g) was added and the mixture heated at 50° C. for 3 hours. The reaction mixture was filtered through Celite, washing with warm DMF (2×100 ml) and concentrated to ca. 100 ml then diluted slowly with water (200 ml) and filtered, drying in vacuo to afford the product as a light brown solid (7.3 g, 68%).

### (g) 6-(Methyloxy)-4-(2H-pyrano[3,2-b]pyridin-7-yl)pyrido[2,3-b]pyrazin-3(4H)-one

2H-Pyrano[3,2-b]pyridin-7-ylboronic acid (1.36 g, 7.69 mmol), 6-(methyloxy)pyrido[2,3-b]pyrazin-3(4H)-one (1.36 g, 7.69 mmol), copper(II) acetate (2.79 g, 15.37 mmol) and triethylamine (2.131 ml, 15.37 mmol) were mixed in dichloromethane (150 ml) and three spatula-fulls of powdered 3 A molecular sieves was added. The mixture was stirred at room temperature under an atmosphere of oxygen overnight. Another 0.68 g of boronic acid and 1.4 g of copper (II) acetate were added and stirring continued. After 6 h, another 0.68 g of boronic acid, 2 ml of triethylamine and 1.4 g of copper (II) acetate were added and stirring continued overnight. The mixture was filtered through Kieselguhr, washed through with dichloromethane, then with 10% methanol/dichloromethane and the filtrate was evaporated. The residue was chromatographed on a 100 g silica column, eluting with 50-100% ethyl acetate/isohexane to give the product (1.56 g, 66%).

MS (+ve ion electrospray) m/z 309 (MH+).

### (h) Title Compound

6-(Methyloxy)-4-(2H-pyrano[3,2-b]pyridin-7-yl)pyrido[2,3-b]pyrazin-3(4H)-one (200 mg, 0.649 mmol) and 1-(2,3-dihydro[1,4]dioxino[2,3-c]pyridin-7-yl)methanamine (for a preparation see Example 3(b) or WO2008009700A1, Example 265) (216 mg, 1.297 mmol) were mixed in isopropanol (8 ml), a solution of hydrogen chloride in 1,4-dioxane (4M, 0.32 ml) was added and the mixture was heated under reflux overnight. Methanol (8 ml) was added to improve solubility and more (2,3-dihydro[1,4]dioxino[2,3-c]pyridin-7-ylmethyl)amine (216 mg, 1.297 mmol) (216 mg, 1.297 mmol) and HCl/1,4-dioxane (0.324 ml, 1.297 mmol) were added. Heating was continued overnight. The solvent was evaporated under vacuum, the residue was dissolved in dichloromethane and aqueous sodium bicarbonate, and the phases were separated. The aqueous phase was extracted with dichloromethane several times, the organic fractions were dried over sodium sulphate and evaporated under vacuum. The crude product was chromatographed on 20 g silica eluted with 0-20% dichloromethane/methanol to give the free base of the title compound (25 mg, 8%).

¹H NMR (CDCl₃) δ 8.27 (1H, s), 8.16 (1H, s), 8.05 (2H, d), 7.15 (1H, s), 6.74 (1H, d), 6.69 (1H, s), 4.67 (1H, d), 4.32 5H, m), 4.15 (1H, t), 3.67 (4H, m), 2.70 (2H, m), 2.54 (1H, m).

MS (+ve ion electrospray) m/z 475 (MH+).

The free base was dissolved in 1-2 ml of chloroform and fumaric acid (1 eq., 6.2 mg, 0.053 mmol) in methanol (1 ml) was added. The solvent was evaporated and the residue was dried under high vacuum to give the fumarate salt (31 mg).

### Example 18

### 4-{(6R/S,7R/S)-7-[(2,3-Dihydro[1,4]dioxino[2,3-c]pyridin-7-ylmethyl)amino]-6-hydroxy-5,6,7,8-tetrahydro-3-quinolinyl}-6-(methyloxy)pyrido[2,3-b]pyrazin-3(4H)-one dihydrochloride

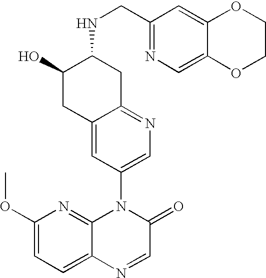

### (a) 1,1-Dimethylethyl [(7R/S)-3-nitro-6-oxo-5,6,7,8-tetrahydro-7-quinolinyl]carbamate

To a solution of Dess-Martin Periodinane (1,1,1-tris(acetyloxy)-1,1-dihydro-1,2-benzodioxol-3-(1H)-one) in dichloromethane (42.2 ml, 12.67 mmol) in dichloromethane (300 ml) at rt was added cis-1,1-dimethylethyl [(6-hydroxy-3-nitro-5,6,7,8-tetrahydro-7-quinolinyl]carbamate (for a preparation see Example 12(c)) (1.96 g, 6.34 mmol) in dichloromethane (150 ml) dropwise over 20 min via a pressure-equalising dropping funnel. The reaction mixture was stirred at rt for 2 h. Additional Dess-Martin Periodinane (4.22 ml, 1.267 mmol) added in one portion and stirring at rt continued for another 2 h. Reaction mixture cautiously poured into 300 mL sat. aq. Na₂S₂O₃/300 mL sat aq. NaHCO₃ and extracted with DCM (200 mL). Organic layer was then washed with 300 mL saturated aq. NaHCO₃, water (250 mL) and brine (250 mL). The organic layer was dried over MgSO₄, filtered and evaporated to give an orange oil. The crude residue was purified by flash column chromatography, eluting with 0-100% EtOAc/Hexane. The relevant fractions were combined and evaporated to deliver an orange oil (932 mg, 3.03 mmol, 48%).

MS (ES+) m/z 252 (MH+-t-Bu⁺).

### (b) trans-1,1-Dimethylethyl[(6R/S,7R/S)-6-hydroxy-3-nitro-5,6,7,8-tetrahydro-7-quinolinyl]carbamate

To a suspension of sodium borohydride (126 mg, 3.33 mmol) in ethanol (25.2 ml) at −78° C. was added a suspension of 1,1-dimethylethyl [(7R/S)-3-nitro-6-oxo-5,6,7,8-tetrahydro-7-quinolinyl]carbamate (0.93 g, 3.03 mmol) in ethanol (50.4 ml). The reaction mixture was stirred at −78° C. for 20 min, warmed to −60° C. then the reaction was quenched at −60° C. with 1 mL saturated aq. NH₄Cl₄ and then warmed to rt. 20 mL MeOH was added and the cloudy orange suspension was stirred at rt for 5 min then concentrated to deliver an orange/white solid. The solid was partially dissolved in DCM (100 mL) and filtered through a silica plug, washing the precipitate extensively with EtOAc (˜300 mL). The filtrate was concentrated to deliver the crude residue. The residue was purified by column chromatography, eluting with 2:1 to 1:1 petrol:ethyl acetate. The relevant fractions were combined and evaporated to deliver a clear, orange oil (379 mg, 1.225 mmol, 41%).

MS (ES+) m/z 254 (MH+-t-Bu⁺).

### (c) trans-1,1-Dimethylethyl [(6R/S,7R/S)-3-amino-6-hydroxy-5,6,7,8-tetrahydro-7-quinolinyl]carbamate

A solution of trans-1,1-dimethylethyl [(6R/S,7R/S)-6-hydroxy-3-nitro-5,6,7,8-tetrahydro-7-quinolinyl]carbamate (1.06 g, 3.43 mmol) in ethanol (50 ml) at rt was added 10% palladium on carbon (0.365 g, 3.43 mmol). The reaction mixture was stirred under an atmosphere of hydrogen at rt overnight. The reaction mixture was purged with argon then filtered through Celite, washing with ethanol (50 mL). The filtrate was evaporated and used directly in the next step without further purification.

MS (ES+) m/z 280 (MH⁺).

### (d) trans-1,1-Dimethylethyl ((6R/S,7R/S)-6-hydroxy-3-{[6-(methyloxy)-3-nitro-2-pyridinyl]amino}-5,6,7,8-tetrahydro-7-quinolinyl)carbamate

To a solution of trans-1,1-dimethylethyl [(6R/S,7R/S)-3-amino-6-hydroxy-5,6,7,8-tetrahydro-7-quinolinyl]carbamate (958 mg, 3.43 mmol) and 2-chloro-6-(methyloxy)-3-nitropyridine (711 mg, 3.77 mmol) in DMF (8.575 ml) at rt was added sodium bicarbonate (576 mg, 6.86 mmol). The reaction mixture was then stirred at 60° C. for 6 h then cooled to rt and left overnight. The reaction was evaporated then partitioned between DCM (50 mL) and water (50 mL) and extracted with DCM (3×50 mL). The combined organics were dried over Na₂SO₄, filtered and evaporated to deliver an orange oil, which was purified by flash column chromatography, eluting with 0-10% MeOH/DCM. The relevant fractions were combined and evaporated to deliver a clear, orange oil (725 mg, 1.68 mmol, 49% over 2 steps).

MS (ES+) m/z 432 (MH⁺).

### (e) trans-1,1-Dimethylethyl ((6R/S,7R/S)-3-{[3-amino-6-(methyloxy)-2-pyridinyl]amino}-6-hydroxy-5,6,7,8-tetrahydro-7-quinolinyl)carbamate

To a solution of trans-1,1-dimethylethyl ((6R/S,7R/S)-6-hydroxy-3-{[6-(methyloxy)-3-nitro-2-pyridinyl]amino}-5,6,7,8-tetrahydro-7-quinolinyl)carbamate (725 mg, 1.680 mmol) in ethanol (25 ml) at rt was added palladium on carbon (179 mg). The reaction mixture was stirred at rt for 3 h. Additional palladium on carbon (100 mg) was added and the reaction was stirred under H₂ overnight. The reaction was purged with argon then filtered through Celite, washing with ethanol (50 mL). The filtrate was evaporated and used directly in the next step without further purification.

MS (ES+) m/z 402 (MH⁺).

### (f) trans-Ethyl N-[2-{[(6R/S,7R/S)-7-({[(1,1-dimethylethyl)oxy]carbonyl}amino)-6-hydroxy-5,6,7,8-tetrahydro-3-quinolinyl]amino}-6-(methyloxy)-3-pyridinyl]glycinate

To a solution of trans-1,1-dimethylethyl ((6R/S,7R/S)-3-{[3-amino-6-(methyloxy)-2-pyridinyl]amino}-6-hydroxy-5,6,7,8-tetrahydro-7-quinolinyl)carbamate (674 mg, 1.679 mmol) and in toluene (40 ml) at rt was added ethyl glyoxalate solution (50% wt. in toluene) (0.4 ml, 1.679 mmol). The reaction mixture was stirred at rt for 2 h. Molecular sieves (3 A) (0.2 g) were added and the reaction was stirred at rt for 72 h. The reaction mixture was diluted with methanol (40.0 ml) and the reaction cooled to 0° C. then sodium borohydride (69.9 mg, 1.847 mmol) was added in one portion After warming to rt, stirring continued for 20 min then the reaction was quenched with water (10 mL) and reaction was concentrated to dryness.

The crude residue was partitioned between ethyl acetate (100 mL) and water (100 mL) and extracted with ethyl acetate (3×100 mL). Combined organic layers washed with brine 9100 mL) dried over Na₂SO₄, filtered and evaporated to afford a dark green oil, which was purified by flash column chromatography, eluting with 0-10% MeOH/DCM. The relevant fractions were combined and evaporated to give an orange foam (417 mg, 0.855 mmol, 51%).

MS (ES+) m/z 488 (MH⁺).

### (g) trans-Ethyl N-[2-{[(6R/S,7R/S)-6-{[(1,1-dimethylethyl)(dimethyl)silyl]oxy}-7-({[(1,1-dimethylethyl)oxy]carbonyl}amino)-5,6,7,8-tetrahydro-3-quinolinyl]amino}-6-(methyloxy)-3-pyridinyl]glycinate

To a solution of trans-ethyl N-[2-{[(6R/S,7R/S)-7-({[(1,1-dimethylethyl)oxy]carbonyl}amino)-6-hydroxy-5,6,7,8-tetrahydro-3-quinolinyl]amino}-6-(methyloxy)-3-pyridinyl]glycinate (417 mg, 0.855 mmol) and imidazole (64.0 mg, 0.941 mmol) in DMF (1.425 mL) at rt was added chloro(1,1-dimethylethyl)dimethylsilane (129 mg, 0.855 mmol). The reaction mixture was stirred at rt overnight. Additional chloro(1,1-dimethylethyl)dimethylsilane (64.5 mg, 0.428 mmol) and additional imidazole (29.1 mg, 0.428 mmol) were added at rt and the reaction was stirred at rt. After 5 h at rt the reaction was quenched with water (20 mL) and extracted with DCM (3×20 mL) and the combined organic layers were washed with water (20 mL) and brine (20 mL), dried over Na₂SO₄, filtered and evaporated to deliver a dark orange oil. The crude residue was purified by flash column chromatography twice, eluting with 0-100% EtOAc/Hexane, then flushing with EtOAc. The relevant fractions were combined and evaporated to deliver an orange oil (205 mg, 0.341 mmol, 40%)

MS (ES+) m/z 602 (MH⁺).

### (h) trans-1,1-Dimethylethyl {(6R/S,7R/S)-6-{[(1,1-dimethylethyl)(dimethyl)silyl]oxy}-3-[6-(methyloxy)-3-oxo-2,3-dihydropyrido[2,3-b]pyrazin-4(1H)-yl]-5,6,7,8-tetrahydro-7-quinolinyl}carbamate

To a suspension of sodium hydride (14.99 mg, 0.375 mmol) in THF (8.516 ml) at 0° C. was added trans-ethyl N-[2-{[(6R/S,7R/S)-6-{[(1,1-dimethylethyl)(dimethyl)silyl]oxy}-7-({[(1,1-dimethylethyl)oxy]carbonyl}amino)-5,6,7,8-tetrahydro-3-quinolinyl]amino}-6-(methyloxy)-3-pyridinyl]glycinate (205 mg, 0.341 mmol) in THF (8.52 ml) dropwise. The reaction mixture was stirred at 0° C. for 15 min, then warmed to rt and stirred at rt for 20 min.

The reaction was quenched with 2 mL saturated NH₄Cl (aq) at 0° C., diluted with ethyl acetate (20 mL), and excess MgSO₄ added. The reaction was stirred vigorously for 10 min at rt then filtered and evaporated to deliver an orange oil which was used in the next step without further purification.

MS (ES+) m/z 556 (MH⁺).

### (i) trans-1,1-Dimethylethyl {(6R/S,7R/S)-6-{[(1,1-dimethylethyl)(dimethyl)silyl]oxy}-3-[6-(methyloxy)-3-oxopyrido[2,3-b]pyrazin-4(3H)-yl]-5,6,7,8-tetrahydro-7-quinolinyl}carbamate

To a solution of trans-1,1-dimethylethyl {(6R/S,7R/S)-6-{[(1,1-dimethylethyl)(dimethyl)silyl]oxy}-3-[6-(methyloxy)-3-oxo-2,3-dihydropyrido[2,3-b]pyrazin-4(1H)-yl]-5,6,7,8-tetrahydro-7-quinolinyl}carbamate (0.340 mmol, assume 100% yield from previous step) in DCM (17.100 ml) at rt was added manganese dioxide (446 mg, 5.13 mmol). The reaction mixture was stirred at rt overnight. The reaction mixture was filtered through a pad of Celite, washing with 500 mL 10% MeOH/DCM. The filtrate was evaporated to deliver a dark orange oil, which was used in the next step without further purification.

MS (ES+) m/z 554 (MH⁺).

### (j) trans-1,1-Dimethylethyl {(6R/S,7R/S)-6-hydroxy-3-[6-(methyloxy)-3-oxopyrido[2,3-b]pyrazin-4(3H)-yl]-5,6,7,8-tetrahydro-7-quinolinyl}carbamate

To a solution of trans-1,1-dimethylethyl {(6R/S,7R/S)-6-{[(1,1-dimethylethyl)(dimethyl)silyl]oxy}-3-[6-(methyloxy)-3-oxopyrido[2,3-b]pyrazin-4(3H)-yl]-5,6,7,8-tetrahydro-7-quinolinyl}carbamate (0.340 mmol, assume 100% yield from previous step) in THF (3.40 ml) at 0° C. was added tetra butylammonium fluoride (1 M in THF) (0.373 ml, 0.373 mmol) dropwise. The reaction mixture was stirred at 0° C. for 5 min then warmed to rt and stirred for 35 min. Reaction was quenched with water (10 mL) and stirred for 10 min at rt then diluted with ethyl acetate (20 mL) and brine (10 mL), the layers were separated and extracted with ethyl acetate (3×30 mL). Combined organics were dried over MgSO₄, filtered and evaporated to deliver a dark orange oil. The crude residue was purified by flash column chromatography, eluting with 4% MeOH/DCM. The relevant fractions were combined and evaporated to deliver an orange powder (53 mg, 0.121 mmol, 36% over 3 steps).

MS (ES+) m/z 440 (MH⁺).

### (k) trans-4-[(6R/S,7R/S)-7-amino-6-hydroxy-5,6,7,8-tetrahydro-3-quinolinyl]-6-(methyloxy)pyrido[2,3-b]pyrazin-3(4H)-one

To a solution of trans-1,1-dimethylethyl {(6R/S,7R/S)-6-hydroxy-3-[6-(methyloxy)-3-oxopyrido[2,3-b]pyrazin-4(3H)-yl]-5,6,7,8-tetrahydro-7-quinolinyl}carbamate (53 mg, 0.121 mmol) in DCM (5 ml) at rt was added hydrochloric acid in 1,4-dioxane (1.0 mL, 4.00 mmol). Reaction mixture changed from clear, orange to a cloudy green suspension. The reaction mixture was stirred at rt for 2 hr. The reaction mixture was evaporated to deliver a pale orange powder which was diluted in the minimum volume of methanol and purified on an SCX cartridge, eluting with 20 mL DCM, 20 mL DCM/MeOH (1:1), 20 mL MeOH, 20 mL (3:1) 2 M NH₃ in methanol/methanol and 20 mL (1:1) 2 M NH₃ in methanol/methanol. The relevant (basic) fractions were evaporated to afford a pale orange powder (40 mg, 98%).

MS (ES+) m/z 340 (MH⁺)

### (l) Title Compound

A solution of trans-4-[(6R/S,7R/S)-7-amino-6-hydroxy-5,6,7,8-tetrahydro-3-quinolinyl]-6-(methyloxy)pyrido[2,3-b]pyrazin-3(4H)-one (41.1 mg, 0.121 mmol) and 2,3-dihydro[1,4]dioxino[2,3-c]pyridine-7-carbaldehyde (for synthesis see WO2004058144 Example 2(c) or WO2003087098 Example 19(d)) (16 mg, 0.097 mmol) in dichloromethane (5 ml) and methanol (0.5 ml) and N,N-Dimethylformamide (0.1 ml) at rt was stirred at rt for 30 min then sodium triacetoxyborohydride (51.3 mg, 0.242 mmol) was added. The reaction mixture was stirred at rt overnight. The reaction mixture was diluted with DCM (10 mL), washed with saturated aq. NaHCO₃ (10 mL) and back-extracted with DCM (20 mL). Combined organics were washed with half-saturated brine/water (5 mL), dried over MgSO₄, filtered and evaporated to deliver an orange oil. The crude residue was purified on silica, eluting with 5-10% MeOH/DCM. Relevant fractions were combined and evaporated to deliver the free base of the title compound as a clear yellow/orange oil (33 mg, 0.07 mmol, 70%).

MS (ES+) m/z 489 (MH⁺).

¹H NMR (400 MHz) δ(CDCl₃) 2.83-3.13 (6H, m), 3.25 (1H, dd), 3.46 (1H, dd), 3.67 (3H, s), 3.85-3.95 (2H, m), 4.27-4.62 (4H, m), 6.72-6.85 (2H, m), 7.45 (1H, d), 8.02-8.13 (2H, m), 8.27 (1H, s), 8.38 (1H, d).

This compound was converted to the di-HCl salt by dissolving the free base in MeOH, adding a large excess of 1 M HCl in MeOH (˜2 mL) and then removing the solvent to give the title compound (30 mg).

MS (ES+) m/z 489 (MH⁺)

### Examples 19 and 20

### 6-[({3-[6-(Methyloxy)-3-oxopyrido[2,3-b]pyrazin-4(3H)-yl]-5,6,7,8-tetrahydro-7-quinolinyl}amino)methyl]-2H-pyrido[3,2-b][1,4]oxazin-3(4H)-one, Enantiomers 1 and 2

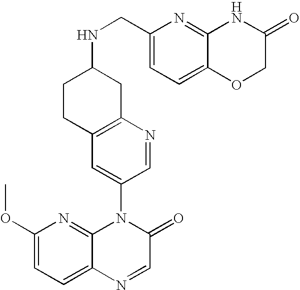

The free base of the racemate of the title compound (for a preparation see Example 6) (30 mg) was chromatographed on a Chiralpak AS-H column eluting with 85% acetonitrile: 15% methanol: 0.1% isopropylamine affording firstly Enantiomer 1 (8 mg, Rt 7.3 mins, 100% ee[alphaD]=−35.04 (c=0.7%, MeOH)) then Enantiomer 2 (6.5 mg, Rt 10.5 mins, 100% ee[alphaD]=+48.7 (c=0.8%, MeOH)).

### Examples 21 and 22

### Cis-6-[({6-Hydroxy-3-[6-(methyloxy)-3-oxopyrido[2,3-b]pyrazin-4(3H)-yl]-5,6,7,8-tetrahydro-7-quinolinyl}amino)methyl]-2H-pyrido[3,2-b][1,4]oxazin-3(4H)-one Enantiomers 1 and 2

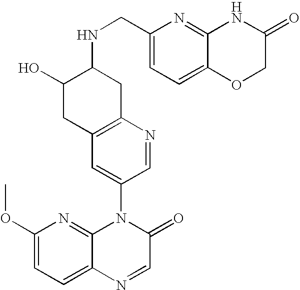

The free base of the racemate of the title compound (for a preparation see Example 12) (53 mg) was chromatographed on a Chiralpak IA column eluting with 40% acetonitrile: 40% methanol: 20% isopropanol: 0.1% isopropylamine affording firstly Enantiomer 1 (16 mg, Rt 7.3 mins, 100% ee) then Enantiomer 2 (16 mg, Rt 16.7 mins, 100% ee).

### Examples 23 and 24

### Cis-4-{7-[(2,3-Dihydro[1,4]dioxino[2,3-c]pyridin-7-ylmethyl)amino]-6-hydroxy-5,6,7,8-tetrahydro-3-quinolinyl}-6-(methyloxy)pyrido[2,3-b]pyrazin-3(4H)-one

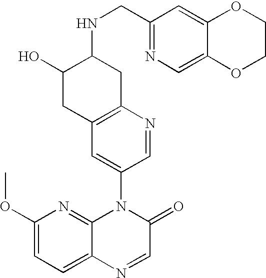

The free base of the racemate of the title compound (for a preparation see Example 14) (50 mg) was chromatographed on a Chiralpak IA column eluting with 40% acetonitrile: 40% methanol: 20% isopropanol: 0.1% isopropylamine affording firstly Enantiomer 1 (10.9 mg, Rt 5.2 mins, 100% ee) then Enantiomer 2 (11 mg, Rt 8.9 mins, 100% ee).

### Example 25

### 4-{7-[(3,4-dihydro-2H-pyrido[3,2-b][1,4]oxazin-6-ylmethyl)amino]-5,6,7,8-tetrahydro-3-quinolinyl}-6-(methyloxy)pyrido[2,3-b]pyrazin-3(4H)-one dihydrochloride

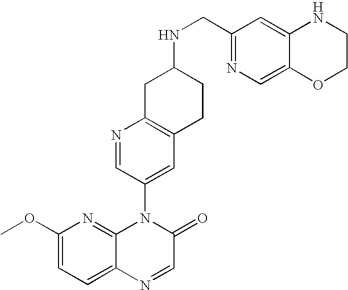

### (a) 1,1-Dimethylethyl 6-[({3-[6-(methyloxy)-3-oxopyrido[2,3-b]pyrazin-4(3H)-yl]-5,6,7,8-tetrahydro-7-quinolinyl}amino)methyl]-2,3-dihydro-4H-pyrido[3,2-b][1,4]oxazine-4-carboxylate

A solution of 1,1-dimethylethyl 7-formyl-2,3-dihydro-1H-pyrido[3,4-b][1,4]oxazine-1-carboxylate (90 mg, 0.34 mmol) (for a synthesis see WO2007/081597 Example (7(f)) and 4-(7-amino-5,6,7,8-tetrahydro-3-quinolinyl)-6-(methyloxy)pyrido[2,3-b]pyrazin-3(4H)-one (for a preparation see Example 6(f)) (110 mg, 0.34 mmol) in DCM/methanol was treated with sodium triacetoxyborohydride (210 mg, 0.99 mmol). After 2 hours the reaction was quenched with saturated aqueous NaHCO₃ solution. The aqueous layer was separated and washed with 5% MeOH in DCM. The organic layers were then combined, dried and the solvent removed. This residue was then chromatographed eluting 0-20% MeOH in DCM gradient to give an oil (0.1 g, 51%).

MS (ES+) m/z 572 (MH⁺).

### (b) Title Compound

A solution of 1,1-dimethylethyl 6-[({3-[6-(methyloxy)-3-oxopyrido[2,3-b]pyrazin-4(3H)-yl]-5,6,7,8-tetrahydro-7-quinolinyl}amino)methyl]-2,3-dihydro-4H-pyrido[3,2-b][1,4]oxazine-4-carboxylate (100 mg) was dissolved in DCM/methanol (10 ml/5 ml) and treated with 1M HCl in 1,4-dioxane. After stirring overnight the mixture was cooled in an ice bath for 1 hour then filtered. The resulting white solid was dried in vacuo (95 mg).

MS (ES+) m/z 472 (MH⁺)

¹H NMR (400 MHz) δ(d6-DMSO) 1.90-2.10 (1H, m), 2.40-2.60 (2H, m), 2.90-3.10 (2H, m), 3.20-3.30 (1H, m), 3.50-3.60 (2H, m), 3.60 (3H, s), 3.70-3.80 (1H, m), 4.20-4.30 (2H, m), 4.40-4.65 (2H, m), 6.90 (1H, d), 7.40 (1H, s), 7.85 (1H, s), 8.20-8.30 (3H, m), 8.55 (1H, s), 9.35 (1H, bs), 10.20-10.50 (2H, b)

### Example 26

### 6-[({2-[6-(methyloxy)-3-oxopyrido[2,3-b]pyrazin-4(3H)-yl]-5,6,7,8-tetrahydro-6-quinazolinyl}amino)methyl]-2H-pyrido[3,2-b][1,4]oxazin-3(4H)-one hydrochloride

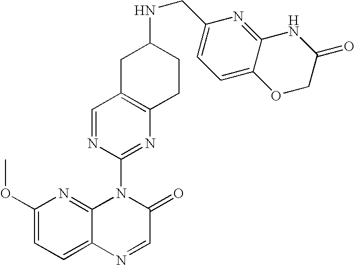

### (a) 6-(Methyloxy)-3-nitro-2-pyridinamine

A solution/suspension of 2-chloro-6-(methyloxy)-3-nitropyridine (65.7 g, 348 mmol) in 2M ammonia in methanol (500 ml, 1000 mmol) and aqueous ammonia (500 ml, 348 mmol) was stirred at 65° C. for 18 h. The reaction was cooled down and the solid filtered off and washed with water (2×100 ml). The solid was dried in the vacuum oven at 40° C. overnight to afford the product as a bright yellow solid (52.14 g, 84% purity by NMR, 74%).

MS (ES+) m/z 170 (MH⁺).

### (b) 6-(Methyloxy)-2,3-pyridinediamine

6-(Methyloxy)-3-nitro-2-pyridinamine (26 g, 129 mmol) was suspended in ethanol (500 ml) at room temperature under argon and then treated with palladium on carbon (15 g, 14.10 mmol) (10% paste). The reaction was stirred under 1 atm of hydrogen overnight. The reaction was filtered through a Celite pad and the pad washed with ethanol (500 ml). Ethanol was evaporated to afford the product as a purple oil (20.68 g, slightly impure).

MS (ES+) m/z 140 (MH⁺).

### (c) Ethyl N-[2-amino-6-(methyloxy)-3-pyridinyl]glycinate

6-(Methyloxy)-2,3-pyridinediamine (21.7 g, 87% purity, 136 mmol) was dissolved in acetonitrile (500 ml) at room temperature under argon and then treated with potassium carbonate (24.38 g, 176 mmol) and ethyl bromoacetate (18.13 ml, 163 mmol). The reaction was stirred at room temperature overnight. The acetonitrile was then removed in vacuo. The reaction was repeated using more 6-(methyloxy)-2,3-pyridinediamine (20.68 g, 87% purity, 129 mmol), in acetonitrile (500 ml), potassium carbonate (25.9 g) and ethyl bromoacetate (23.23 g) and the reaction was again stirred at room temperature overnight and the acetonitrile was then removed in vacuo. The residues were partitioned between water (1 L) and ethyl acetate (1 L) and the layers separated. The aqueous layer was extracted once more with ethyl acetate (1 L) and the combined organics were dried MgSO₄, filtered and evaporated to afford a purple oil (64 g). The oil was treated with DCM (300 ml) and the insoluble impurities filtered off. The DCM solution was loaded onto a 800 g silica column and eluted with 0-2% MeOH/DCM to afford 40.6 g of desired product as a solid (LCMS and NMR consistent with 75% desired product with 15% cyclized product 6-(methyloxy)-1,4-dihydropyrido[2,3-b]pyrazin-3(2H)-one).

MS (ES+) m/z 226 (MH⁺).

### (d) 6-(Methyloxy)-1,4-dihydropyrido[2,3-b]pyrazin-3(2H)-one

Ethyl N-[2-amino-6-(methyloxy)-3-pyridinyl]glycinate (40.6 g, 135 mmol) was dissolved in THF (1 L) at room temperature under argon and treated with potassium tert-butoxide (15.17 g, 135 mmol). After 2 h at room temperature saturated NH₄Cl (500 ml) was added and the THF evaporated. Water (500 ml) was added followed by 20% MeOH/DCM (1 L); the insolubles were filtered off, washed with diethyl ether and dried in the vacuum oven at 40° C. overnight to afford the desired product as a yellow solid (15.3 g). The two phases were transferred to a separating funnel and separated. The aqueous layer was extracted twice more with 20% MeOH/DCM (2×500 ml) and the combined organics were dried over MgSO₄, filtered and evaporated to afford a brown solid which was washed with plenty of diethyl ether to afford more of the desired product as a pale green solid (7.7 g): LCMS and NMR consistent with product (20% of oxidized material present by NMR).

MS (ES+) m/z 180 (MH⁺).

### Alternative Procedure:

Ethyl N-[2-amino-6-(methyloxy)-3-pyridinyl]glycinate (16.2 g, 72 mmol) was dissolved in tetrahydrofuran (500 ml) and cooled to 0° C. (ice bath cooling) under argon. This was then treated with potassium tert-butoxide (1M in THF, 80 ml, 80 mmol). After 1.5 h the reaction was treated with acetic acid (80 mmol) and evaporated to give a dark solid. This was triturated with water (200 ml), filtered and dried in vacuo (˜13 g, quant.), which was used without further purification

### (e) Phenylmethyl 6-(methyloxy)-3-oxo-3,4-dihydropyrido[2,3-b]pyrazine-1(2H)-carboxylate

To 6-(methyloxy)-1,4-dihydropyrido[2,3-b]pyrazin-3(2H)-one (6.35 g, 35.4 mmol) in ethyl acetate (600 ml)/sodium bicarbonate (saturated solution) (200 ml) stirred vigorously was added at room temperature benzyl chloroformate (5.31 ml, 37.2 mmol). After 45 minutes the reaction was complete. The layers were separated and the organic layer was dried on magnesium sulphate, filtered and evaporated to afford the desired product as an off-white solid (11 g, 99%).

MS (ES+) m/z 314 (MH⁺).

### (f) 1,1-Dimethylethyl (2-amino-5,6,7,8-tetrahydro-6-quinazolinyl)carbamate

Tris(dimethylamino)methane (12.19 ml, 70.3 mmol) and 1,1-dimethylethyl (4-oxocyclohexyl)carbamate (3.0 g, 14.07 mmol) in toluene (100 mL) was heated for 4 hours at 90° C. The toluene was removed under reduced pressure and the residue dissolved in EtOH (125 mL). Guanidine carbonate (6.34 g, 35.2 mmol) was added and the solution heated to reflux for 4 hours, then allowed to cool to room temperature and stirred overnight. The solvent was evaporated and the residue was diluted with DCM. The organic was washed with brine and the aqueous back-extracted with fresh DCM. The organics were combined, dried over Na₂SO₄, filtered, and concentrated under reduced pressure. The crude material was chromatographed using a gradient of 0-100% (DCM/MeOH/NH4OH) (90:10:1) in DCM. Further chromatography was performed using a gradient of 0-100% acetone/CHCl₃ to yield a bright yellow foamy solid (1.234 g, 33%).

MS (ES+) m/z 265 (MH⁺).

### (g) 1,1-Dimethylethyl (2-iodo-5,6,7,8-tetrahydro-6-quinazolinyl)carbamate

1,1-Dimethylethyl (2-amino-5,6,7,8-tetrahydro-6-quinazolinyl)carbamate (0.250 g, 0.946 mmol), copper(I) iodide (0.180 g, 0.946 mmol), diiodomethane (0.389 ml, 4.82 mmol) and isoamyl nitrate (0.382 ml, 2.84 mmol) in THF (10 ml) was heated to reflux under nitrogen for 3 hours. The reaction was cooled to ambient temperature and the mixture partitioned between EtOAc and 1N HCl solution. The aqueous layer was then extracted with EtOAc (twice) and the organic layers combined. The solution was dried over Na₂SO₄, filtered, and concentrated under reduced pressure. The crude material was chromatographed using a gradient of 0-100% EtOAc/Hexanes to yield a yellow oil (147 mg, 41%).

MS (ES+) m/z 376 (MH⁺).

### (h) Phenylmethyl 4-[6-({[(1,1-dimethylethyl)oxy]carbonyl}amino)-5,6,7,8-tetrahydro-2-quinazolinyl]-6-(methyloxy)-3-oxo-3,4-dihydropyrido[2,3-b]pyrazine-1(2H)-carboxylate

In a sealed tube was combined 1,1-dimethylethyl (2-iodo-5,6,7,8-tetrahydro-6-quinazolinyl)carbamate) (925 mg, 2.46 mmol), phenylmethyl 6-(methyloxy)-3-oxo-3,4-dihydropyrido[2,3-b]pyrazine-1(2H)-carboxylate (386 mg, 1.23 mmol), 1,2 diaminocyloxehylamine (28 mg, 0.246 mmol), copper idodide (I) (47 mg, 0.246 mmol) and potassium carbonate (358 mg, 2.59 mmol) in 1,4-dioxane (15 mL). The combined mixture was heated at 125° C. for 10 hours. The solvent removed in vacuum and the residue purified by chromatography eluting with 0-10% MeOH/DCM gave a solid (270 mg, 39% yield).

MS (ES+) m/z 561 (MH⁺).

### (i) 1,1-Dimethylethyl {2-[6-(methyloxy)-3-oxo-2,3-dihydropyrido[2,3-b]pyrazin-4(1H)-yl]-5,6,7,8-tetrahydro-6-quinazolinyl}carbamate

Phenylmethyl 4-[6-({[(1,1-dimethylethyl)oxy]carbonyl}amino)-5,6,7,8-tetrahydro-2-quinazolinyl]-6-(methyloxy)-3-oxo-3,4-dihydropyrido[2,3-b]pyrazine-1(2H)-carboxylate (270 mg, 0.482 mmol) in ethyl acetate (10 ml) was treated with Pd/C (20 mg, 0.188 mmol) and hydrogenated under 60 psi of hydrogen for 10 hours. The mixture was filtered and evaporated to give a solid (180 mg, 88% yield).

MS (ES+) m/z 427 (MH⁺).

### (j) 1,1-Dimethylethyl {2-[6-(methyloxy)-3-oxopyrido[2,3-b]pyrazin-4(3H)-yl]-5,6,7,8-tetrahydro-6-quinazolinyl}carbamate

A solution of 1,1-dimethylethyl {2-[6-(methyloxy)-3-oxo-2,3-dihydropyrido[2,3-b]pyrazin-4(1H)-yl]-5,6,7,8-tetrahydro-6-quinazolinyl}carbamate (180 mg, 0.422 mmol) in DCM (15 ml) was treated with manganese(II) oxide (183 mg, 2.1 mmol). The mixture stirred for 1 hour then filtered and evaporated to give a solid (120 mg, 67% yield).

MS (ES+) m/z 425 (MH⁺).

### (k) 4-(6-Amino-5,6,7,8-tetrahydro-2-quinazolinyl)-6-(methyloxy)pyrido[2,3-b]pyrazin-3(4H)-one hydrochloride

To 1,1-dimethylethyl {2-[6-(methyloxy)-3-oxopyrido[2,3-b]pyrazin-4(3H)-yl]-5,6,7,8-tetrahydro-6-quinazolinyl}carbamate (120 mg, 0.283 mmol) in DCM (10 ml) was added hydrochloric acid in 1,4-dioxane (4M; 0.25 mmol, 1 mmol). The mixture was stirred for 18 hours. The solvent was removed in vacuum to give a solid (92 mg, 0.284 mmol, 100% yield).

MS (ES+) m/z 325 (MH⁺).

### (l) Title Compound

A mixture of 4-(6-amino-5,6,7,8-tetrahydro-2-quinazolinyl)-6-(methyloxy)pyrido[2,3-b]pyrazin-3(4H)-one hydrochloride (46 mg, 0.142 mmol), 3-oxo-3,4-dihydro-2H-pyrido[3,2-b][1,4]oxazine-6-carbaldehyde (for a synthesis see WO03087098 Example 31(e)) (25 mg, 0.142 mmol), and sodium bicarbonate (119 mg, 1.42 mmol) in DCM (4 ml) and methanol (0.8 ml) was treated with sodium sulfate (200 mg, 1.408 mmol) and stirred overnight. Sodium triacetoxyborohydride (90 mg, 0.425 mmol) was added and the reaction was stirred under nitrogen for 4 hours. The mixture was then chromatographed using 0-20% MeOH/DCM (containing 1% NH₄OH) giving the free base of the title compound a solid (18 mg, 22%).

¹H NMR δH CDCl₃, (250 MHz) 1.8-2.25 (m, 4H), 2.8 (m, 1H), 3.0-3.3 (m, 4H), 3.6 (s, 3H), 3.95 (s, 2H), 4.65 (s, 2H), 6.7 (d, 1H), 7.0 (bs, 1H), 7.2 (d, 1H), 8.1 (d, 1H), 8.25 (s, 1H), 8.7 (s, 1H)

This material was converted into the title compound by addition of 1 equivalents of 1M HCl in ether to the solution of the compound in DCM followed by evaporation to dryness.

### Example 27

### 4-{6-[(2,3-Dihydro[1,4]dioxino[2,3-c]pyridin-7-ylmethyl)amino]-5,6,7,8-tetrahydro-2-quinazolinyl}-6-(methyloxy)pyrido[2,3-b]pyrazin-3(4H)-one hydrochloride

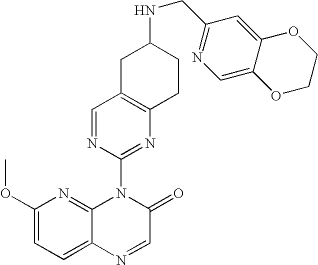

A mixture of 4-(6-amino-5,6,7,8-tetrahydro-2-quinazolinyl)-6-(methyloxy)pyrido[2,3-b]pyrazin-3(4H)-one hydrochloride (for a preparation see Example 26(k)) (46 mg, 0.142 mmol), 2,3-dihydro[1,4]dioxino[2,3-c]pyridine-7-carbaldehyde (for synthesis see WO2004058144 Example 2(c) or WO2003087098 Example 19(d)) (23.4 mg, 0.142 mmol), and sodium bicarbonate (119 mg, 1.42 mmol) in DCM (4 ml) and methanol (0.8 ml) was treated with sodium sulfate (200 mg, 1.408 mmol) and stirred overnight. Sodium triacetoxyborohydride (90 mg, 0.425 mmol) was added and the reaction was stirred under nitrogen for 4 hours. The mixture was then absorbed onto silica and chromatographed using 0-20% MeOH/DCM (containing 1% NH₄OH) giving the free base of the title compound a solid (18 mg, 25%).

¹H NMR δH CDCl₃, (250 MHz) 1.8-2.25 (m, 4H), 2.8 (m, 1H), 3.0-3.3 (m, 4H), 3.6 (s, 3H), 3.95 (s, 2H), 4.3 (dd, 2H), 4.35 (dd, 2H), 6.7 (d, 1H), 6.8 (bs, 1H), 8.1 (d, 1H), 8.15 (d, 1H), 8.25 (s, 1H), 8.7 (s, 1H).

MS (ES+) m/z 510.4 (MH⁺).

This material was converted into the title compound by addition of 1 equivalents of 1M HCl in ether to the solution of the compound in DCM followed by evaporation to dryness.

### Examples 28 and 29

### 6-{[({6-[6-(Methyloxy)-3-oxopyrido[2,3-b]pyrazin-4(3H)-yl]-1,2,3,4-tetrahydro-1-naphthalenyl}methyl)amino]methyl}-2H-pyrido[3,2-b][1,4]oxazin-3(4H)-one Enantiomers E1 and E2, hydrochloride

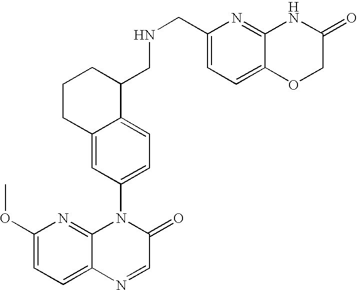

Racemic 4-[5-(aminomethyl)-5,6,7,8-tetrahydro-2-naphthalenyl]-6-(methyloxy)pyrido-[2,3-b]pyrazin-3(4H)-one (for a preparation see Example 9(k)) was resolved by high pressure chromatography using a Chiralpak AS-H column eluting with 0.1% isopropylamine in methanol, affording the E1 enantiomer (Rt 4.0 minutes) then the E2 enantiomer (Rt 6.2 minutes) providing approximately 400 mg of each from 1 g of racemate. The resolved enantiomers were transformed by the procedure generally described in 9(1) to give the title enantiomers E1 and E2, hydrochloride salts.

### Example 30

### 2-{[({3-[6-(Methyloxy)-3-oxopyrido[2,3-b]pyrazin-4(3H)-yl]-7,8-dihydro-5H-pyrano[4,3-b]pyridin-8-yl}methyl)amino]methyl}-5,8-dihydropyrido[2,3-d]pyrimidin-7(6H)-one dihydrochloride

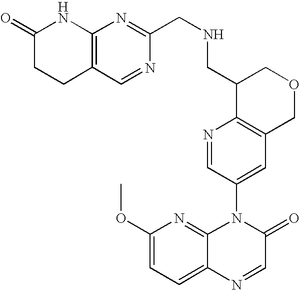

### (a) 3-{[bis(Phenylmethyl)amino]methyl}tetrahydro-4H-pyran-4-one

Dibenzylamine (18.27 ml, 95 mmol) was placed in a round bottom flask at room temperature under argon then 1M HCl in MeOH (95 ml, 95 mmol) was added. The solvent was removed under vacuum to give a white solid. This solid was then re-dissolved in ethanol (200 ml) at room temperature under argon, then paraformaldehyde (8.56 g, 285 mmol) and tetrahydro-4H-pyran-4-one (17.56 ml, 190 mmol) were added. The reaction was heated at 50° C. for 9 hours. The reaction was cooled to room temperature, filtered and the solvent was removed to give a yellow/white oily solid. This solid was triturated twice with Et₂O. The remaining pale yellow solid was dried to give the desired product (28.55 g, 87%) contaminated with some dibenzylamine (˜20%).

MS (ES+) m/z 310 (MH⁺).

### (b) 1-(3-Nitro-7,8-dihydro-5H-pyrano[4,3-b]pyridin-8-yl)-N,N-bis(phenylmethyl)methanamine

To a solution of 3-{[bis(phenylmethyl)amino]methyl}tetrahydro-4H-pyran-4-one (28.55 g, 83 mmol) in methanol (250 ml) was added triethylamine (11.51 ml, 83 mmol) at room temperature under argon. The reaction stirred for 10 mins before 2M ammonia in MeOH (250 ml, 500 mmol) was stirred at room temperature under argon where 1-methyl-3,5-dinitro-2(1H)-pyridinone (16.44 g, 83 mmol) was added in one portion. The reaction was then heated to 65° C. for 2.5 hours. The reaction was then cooled room temperature and concentrated to an orange solid, which was chromatographed eluting with 3×1 L hexane, 4×1 L 25% EtOAc in hexane followed by 4 xX 1 L 50% EtOAc in hexane to give a yellow solid (13.74 g, 34%) still containing some dibenzylamine (˜20%) from the previous step.

MS (ES+) m/z 390 (MH⁺).

### (c) 1,1-Dimethylethyl [(3-amino-7,8-dihydro-5H-pyrano[4,3-b]pyridin-8-yl)methyl]carbamate

A mixture of 1-(3-nitro-7,8-dihydro-5H-pyrano[4,3-b]pyridin-8-yl)-N,N-bis(phenylmethyl)methanamine (13.74 g, 35.3 mmol) in ethanol (300 ml) and acetic acid (100 ml) under argon at room temperature was treated with 10% Pd/C (10 g, 94 mmol) and the reaction was then left to stir over overnight at room temperature under 1 atmosphere of H₂. Filtration and evaporation gave 8-{[(phenylmethyl)amino]methyl}-7,8-dihydro-5H-pyrano[4,3-b]pyridin-3-amine (9.51 g). This material was dissolved in the more ethanol/acetic acid at room temperature. Palladium hydroxide on carbon (4 g, 28.5 mmol) was then added and the resulting mixture was hydrogenated for 2 days. The mixture was filtered, evaporated and the residue was partitioned between ethyl acetate/saturated aqueous sodium bicarbonate solution (˜500 ml of each). The desired product remained in the aqueous phase. The ethyl acetate fraction was discarded and the saturated aqueous sodium bicarbonate solution (ca 500 ml volume) containing ca. 35 mmol of 8-(aminomethyl)-7,8-dihydro-5H-pyrano[4,3-b]pyridin-3-amine was treated with a solution of di-tert-butyl dicarbonate (7.70 g, 35.3 mmol) in 1,4-dioxane (200 ml). After stirring overnight very little reaction had occurred, but on evaporating to dryness the reaction was complete. The solid residue was extracted with dichloromethane (100 ml) and the solution added to a silica column eluting 0-50% ethyl acetate in hexane affording the product as a yellow solid (3.91 g, 40% over three steps).

MS (ES+) m/z 280 (MH⁺).

### (d) 1,1-Dimethylethyl [(3-{[6-(methyloxy)-3-nitro-2-pyridinyl]amino}-7,8-dihydro-5H-pyrano[4,3-b]pyridin-8-yl)methyl]carbamate

To a solution of 1,1-dimethylethyl [(3-amino-7,8-dihydro-5H-pyrano[4,3-b]pyridin-8-yl)methyl]carbamate (3.91 g, 14.00 mmol) in DMF at room temperature under argon was added 2-chloro-6-(methyloxy)-3-nitropyridine (2.64 g, 14.00 mmol) then sodium bicarbonate (2.352 g, 28.0 mmol). The resulting solution was heated to 60° C. overnight, then at 90° C. for 6 hours. The reaction was concentrated and then diluted with DCM (50 ml) and H₂O (50 ml). The aqueous layer was separated and washed a further three times with DCM (50 ml). The organic layers were combined, dried (Na₂SO₄), filtered and the solvent removed to give a yellow oil (6.6 g). This residue was then split into two and purified by chromatography eluting with 0-100% EtOAc in hexane then 0%-20% MeOH in EtOAc affording a yellow oil (4.32 g, 72%).

MS (ES+) m/z 432 (MH⁺).

### (e) 1,1-Dimethylethyl [(3-{[6-(methyloxy)-3-nitro-2-pyridinyl]amino}-7,8-dihydro-5H-pyrano[4,3-b]pyridin-8-yl)methyl]carbamate

1,1-Dimethylethyl [(3-{[6-(methyloxy)-3-nitro-2-pyridinyl]amino}-7,8-dihydro-5H-pyrano[4,3-b]pyridin-8-yl)methyl]carbamate (4.32 g, 10.01 mmol) was dissolved in ethanol (150 ml) and dichloromethane (150 ml) under argon at room temperature. 10% Pd/C (2.5 g) was then added and the reaction was then left to stir for 2 hours at room temperature under 1 atmosphere of H₂. The reaction mixture was then filtered through Celite and the solvent removed to give the desired product (4.10 g, ca 100%).

MS (ES+) m/z 402 (MH⁺).

### (f) Ethyl N-[2-({8-[({[(1,1-dimethylethyl)oxy]carbonyl}amino)methyl]-7,8-dihydro-5H-pyrano[4,3-b]pyridin-3-yl}amino)-6-(methyloxy)-3-pyridinyl]glycinate

1,1-Dimethylethyl [(3-{[3-amino-6-(methyloxy)-2-pyridinyl]amino}-7,8-dihydro-5H-pyrano[4,3-b]pyridin-8-yl)methyl]carbamate (4.10 g, ca 10 mmol) was treated with toluene (100 ml) and dichloromethane (50 ml) at room temperature under argon. Ethyl glyoxalate (2.2 ml, 11.23 mmol) was then added dropwise and the reaction was then left overnight. A further 0.2 eq of ethyl glyoxalate (0.45 ml, 2.25 mmol) was added. After 1 hour methanol (100 ml) was then added and the mixture cooled to 0° C., whereupon sodium borohydride (0.425 g, 11.23 mmol) was added portionwise. After 20 mins at 0° C., water (20 ml) was then added and the MeOH was the removed under vacuum. The aqueous layer was then separated and washed a further three time with EtOAc (20 ml). The organic layers were combined, dried (Na₂SO₄), filtered and the solvent removed to give a black oil (6.7 g). This residue was then split into two and purified using column chromatography eluting with 0-100% EtOAc in hexane and then 0-30% MeOH in EtOAc. This give the desired product as a yellow oil (2.637 g, 53%) and second fraction as a black oil (1.964 g) that contained mainly desired product together with some (20%) of the corresponding methyl ester. Both fractions were combined for the next step.

MS (ES+) m/z 488 (MH⁺).

### (g) 1,1-Dimethylethyl ({3-[6-(methyloxy)-3-oxopyrido[2,3-b]pyrazin-4(3H)-yl]-7,8-dihydro-5H-pyrano[4,3-b]pyridin-8-yl}methyl)carbamate

Sodium hydride (0.361 g, 9.02 mmol) was added to THF (200 ml) at 0° C. under argon, followed by a solution of ethyl N-[2-({8-[({[(1,1-dimethylethyl)oxy]carbonyl}amino)methyl]-7,8-dihydro-5H-pyrano[4,3-b]pyridin-3-yl}amino)-6-(methyloxy)-3-pyridinyl]glycinate (4.40 g, 9.02 mmol) in tetrahydrofuran (200 ml) added dropwise. (During addition there was a colour change from clear to blue to dark blue). After 5 mins at 0° C. the reaction was quenched with the minimum volume of saturated. aqueous NH₄Cl (40 ml). The reaction was then warmed to room temperature where it was diluted with 200 ml EtOAc and an excess of Na₂SO₄ was added (to dry the reaction). The resulting suspension was then stirred vigorously for 5 mins, then filtered and concentrated to give a brown oil (11 g) with spectroscopic data consistent with 1,1-dimethylethyl ({3-[6-(methyloxy)-3-oxo-2,3-dihydropyrido[2,3-b]pyrazin-4(1H)-yl]-7,8-dihydro-5H-pyrano[4,3-b]pyridin-8-yl}methyl)carbamate. This residue was dissolved in DCM (100 ml) at room temperature under argon, then manganese dioxide (7.84 g, 90 mmol) was added portionwise. After 30 mins, the reaction was filtered through Celite and the residue was washed further with DCM (100 ml). The organic layers were combined and the solvent was removed to give a black solid (3.65 g). This residue was then chromatographed eluting with 0-100% EtOAc in hexane followed by 0-20% MeOH in EtOAc, to give the desired product as a yellow oil (1.95 g, 49%).

MS (ES+) m/z 440 (MH⁺).

### (h) 4-[8-(aminomethyl)-7,8-dihydro-5H-pyrano[4,3-b]pyridin-3-yl]-6-(methyloxy)pyrido[2,3-b]pyrazin-3(4H)-one

1,1-Dimethylethyl ({3-[6-(methyloxy)-3-oxopyrido[2,3-b]pyrazin-4(3H)-yl]-7,8-dihydro-5H-pyrano[4,3-b]pyridin-8-yl}methyl)carbamate (1.95 g, 4.44 mmol) was dissolved in DCM (10 ml) at room temperature under argon, where upon TFA (5 ml) was added dropwise. After 1 hour, the solvent was removed. The yellow residue was re-dissolved in 1:1 MeOH/DCM (20 ml) then MP-Carbonate resin (˜10 g) was added until pH8 was attained. The reaction was filtered and the solvent was removed to give the desired product as a pink oily solid (2.49 g, 165%).

MS (ES+) m/z 340 (MH⁺).

### (i) Title Compound

4-[8-(Aminomethyl)-7,8-dihydro-5H-pyrano[4,3-b]pyridin-3-yl]-6-(methyloxy)pyrido[2,3-b]pyrazin-3(4H)-one (0.05 g, 0.147 mmol) and 7-oxo-5,6,7,8-tetrahydropyrido[2,3-d]pyrimidine-2-carbaldehyde (for a synthesis see Preparation A) (0.026 g, 0.147 mmol) were dissolved in chloroform (2 ml) and methanol (0.2 ml) at room temperature under argon, whereupon sodium triacetoxyborohydride (0.094 g, 0.442 mmol) was added portionwise. After 2 hours, the reaction was quenched with saturated NaHCO₃ (5 ml) and diluted with 10% MeOH in DCM (5 ml). The aqueous phase was separated and washed a further 3 times with 10% MeOH in DCM (5 ml). The organic layers were combined, dried (Na₂SO₄), filtered and the solvent removed to give a yellow oil (0.053 g). This residue was then chromatographed eluting with 0-30% MeOH in DCM, to give the free base of the title compound as a clear oil (0.0259 g, 37%).

MS (ES+) m/z 472 (MH⁺).

¹H NMR (400 MHz) δ(MeOD) 2.67 (2H, t), 2.96 (2H, t), 3.01-3.19 (3H, m), 3.63 (3H, s), 3.8-4.00 (2H, m), 4.08 (1H, dd), 4.23 (1H, dd), 4.82-4.86 (1H, m), 6.81 (1H, d), 7.63-7.65 (1H, m), 8.13 (1H, d), 8.19 (1H, s), 8.36 (1H, s) and 8.46-8.48 (1H, m)

This compound was converted to the title compound by dissolving the obtained free base in MeOH (2 ml) whereupon (0.16 ml, 2 eq.) of 1M HCl in MeOH was added. This solution was then evaporated to dryness to give the product (0.030 g).

### Example 31

### 6-{[({3-[6-(methyloxy)-3-oxopyrido[2,3-b]pyrazin-4(3H)-yl]-7,8-dihydro-5H-pyrano[4,3-b]pyridin-8-yl}methyl)amino]methyl}-2H-pyrido[3,2-b][1,4]oxazin-3(4H)-one, trihydrochloride

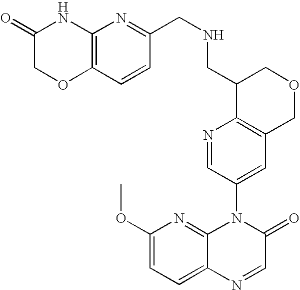

4-[8-(Aminomethyl)-7,8-dihydro-5H-pyrano[4,3-b]pyridin-3-yl]-6-(methyloxy)pyrido[2,3-b]pyrazin-3(4H)-one (for a preparation see Example 30(h))(0.05 g, 0.147 mmol) and 3-oxo-3,4-dihydro-2H-pyrido[3,2-b][1,4]oxazine-6-carbaldehyde (for a synthesis see WO03087098 Example 31(e)) (0.026 g, 0.147 mmol) were dissolved in chloroform (2 ml) and methanol (0.2 ml) at room temperature under argon, whereupon sodium triacetoxyborohydride (0.094 g, 0.442 mmol) was added portionwise. After 2 hours, the reaction was quenched with saturated NaHCO₃ (5 ml) and diluted with 10% MeOH in DCM (5 ml). The aqueous phase was separated and washed a further 3 times with 10% MeOH in DCM (5 ml). The organic layers were combined, dried (Na₂SO₄), filtered and the solvent removed to give a yellow oil (0.053 g). This residue was then chromatographed eluting with 0-30% MeOH in DCM, to give the free base of the title compound as a clear oil (0.044 g, 60%).

MS (ES+) m/z 502 (MH⁺).

¹H NMR (400 MHz) δ(MeOD) 2.9-3.08 (2H, m), 3.11-3.18 (1H, m), 3.62 (3H, s), 3.83 (2H), 4.07 (1H, dd), 4.18 (1H, dd), 4.61 (2H, s), 4.83 (2H, d), 6.80 (1H, d), 6.96 (1H, d), 7.24 (1H, d), 7.62 (1H, d), 8.13 (2H, d), 8.19 (1H, s) and 8.48 (1H, d).

This compound was converted to the title compound by dissolving the obtained free base in MeOH (2 ml) whereupon (0.26 ml, 3 eq.) of 1M HCl in MeOH was added. This solution was then evaporated to dryness to give the product (0.044 g).

### Example 32

### 1-{7-[(6,7-Dihydro[1,4]dioxino[2,3-c]pyridazin-3-ylmethyl)amino]-5,6,7,8-tetrahydro-3-quinolinyl}-7-(methyloxy)pyrido[2,3-b]pyrazin-2(1H)-one

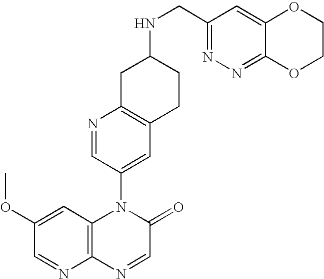

### (a) 3,5-Difluoro-2-nitropyridine

Concentrated sulphuric acid (37.5 ml) was cooled to 4° C. under argon and treated with hydrogen peroxide (20 ml) drop wise over 1.5 hours keeping the temperature below 15° C. The reaction went from yellow to colourless and became very thick and syrup-like. The reaction was cooled back to 4° C. and then treated with a solution of 3,5-difluoro-2-pyridinamine (3.1 g, 23.83 mmol) in sulphuric acid (37.5 ml) drop wise over 15 minutes keeping the temperature below 15° C. After stirring for a further 10 minutes the cooling bath was removed and the yellow solution was stirred under argon for 1 hour during which time it went green and the temperature rose to 30° C. The reaction was cooled to 20° C. and then the cooling bath was removed and reaction mixture was stirred at room temperature for 18 hours to give a thick yellow solution. The reaction mixture was poured into 400 ml ice and slowly basified with solid sodium bicarbonate. A sticky cream solid formed but also a thick inorganic precipitate formed so mixture was then basified with saturated aqueous sodium bicarbonate solution to pH8. The total volume was 1.5 litres and still contained some inorganic material. The mixture was extracted with ethyl acetate (2×250 ml). The organic extracts were combined and washed with saturated brine (100 ml), dried over anhydrous sodium sulphate, filtered and evaporated to dryness. The resulting dark oil (2.51 g) was purified by chromatography eluting with a 0 to 50% ethyl acetate in hexane gradient. The product fractions were combined and evaporated to give a yellow oil, 1.04 g, 27%.

NMR (CDCl₃).7.57 (1H, m), 8.32 (1H, d).

### (b) 1,1-Dimethylethyl {3-[(5-fluoro-2-nitro-3-pyridinyl)amino]-5,6,7,8-tetrahydro-7-quinolinyl}carbamate

In a 50 ml round-bottomed flask 3,5-difluoro-2-nitropyridine (577 mg, 3.61 mmol) was dissolved in anhydrous N,N-dimethylformamide (10 ml) to give a brown solution. 1,1-Dimethylethyl (3-amino-5,6,7,8-tetrahydro-7-quinolinyl)carbamate (for a preparation see Example 6(a)) (950 mg, 3.61 mmol) was added and the brown solution was heated at 70° C. under argon for 21 hours to give a very dark reaction mixture which was evaporated to dryness. The residue was portioned between dichloromethane (100 ml) and saturated aqueous sodium hydrogen carbonate solution (50 ml). The layers were separated and the aqueous layer was washed with dichloromethane (2×25 ml). The organic extract were combined and washed with saturated brine (25 ml), passed through a hydrophobic frit and evaporated to dryness and the residue was chromatographed eluting with a 20 to 100% ethyl acetate in iso-hexane gradient. The product fractions were combined to give a yellow solid, 472 mg, 32%.

MS (ES+) m/z 348 (M-^{t}Bu).

NMR (CDCl₃) 1.48 (9H, bs), 1.79, (1H, m), 2.20 (1H, m), 2.78-2.92 (3H, m), 3.33 (1H, m), 4.11 (1H, m), 4.61 (1H, m), 7.15 (1H, m), 7.35 (1H, d), 7.86 (1H, m), 8.39 (1H, m), 9.24 (1H, d).

### (c) 1,1-Dimethylethyl (3-{[5-(methyloxy)-2-nitro-3-pyridinyl]amino}-5,6,7,8-tetrahydro-7-quinolinyl)carbamate

A suspension of 1,1-dimethylethyl {3-[(5-fluoro-2-nitro-3-pyridinyl)amino]-5,6,7,8-tetrahydro-7-quinolinyl}carbamate (470 mg, 1.165 mmol) in anhydrous methanol (20 ml) was stirred at room temperature under argon. A 25 wt % solution of sodium methoxide (0.55 ml, 2.405 mmol) in methanol was added drop wise over 5 minutes. The solid gradually started to dissolve and a deep red/orange colour formed. The reaction was stirred at room temperature for 15 minutes until all the starting material dissolved. The reaction mixture was evaporated to near dryness (no heat used) and the red gum was partitioned between dichloromethane (50 ml) and water (25 ml) and the deep red colour disappeared leaving a yellow mixture. Saturated brine (25 ml) was added and the mixture was stirred vigorously for 10 minutes and then the layers were separated and the aqueous layer was washed with dichloromethane (25 ml). The organic extracts were combined and washed with saturated brine (25 ml), passed through a hydrophobic frit and evaporated to deep red oil which was chromatographed eluting with a 20 to 100% ethyl acetate in iso-hexane gradient. The product fractions were combined and evaporated to give a dark yellow foam (528 mg, 109%).

MS (ES+) m/z 416.

### (d) 1,1-Dimethylethyl (3-{[2-amino-5-(methyloxy)-3-pyridinyl]amino}-5,6,7,8-tetrahydro-7-quinolinyl)carbamate

A solution of 1,1-dimethylethyl (3-{[5-(methyloxy)-2-nitro-3-pyridinyl]amino}-5,6,7,8-tetrahydro-7-quinolinyl)carbamate (1.77 g, 4.26 mmol) in ethanol (150 ml) was hydrogenated at atmospheric pressure in the presence of 10% palladium on carbon (55% water) (500 mg) for 19 hours and the reaction mixture was filtered through Kieselguhr and washed through with ethanol (150 ml). The filtrate went from pale yellow to dark brown and was evaporated to dryness to give the title compound as a dark brown oil, 2.28 g. Contains ˜30% w/w ethanol

MS (ES+) m/z 386

This material was used directly in the next step.

### (e) Ethyl N-[3-{[7-({[(1,1-dimethylethyl)oxy]carbonyl}amino)-5,6,7,8-tetrahydro-3-quinolinyl]amino}-5-(methyloxy)-2-pyridinyl]glycinate

A dark brown solution of 1,1-dimethylethyl (3-{[2-amino-5-(methyloxy)-3-pyridinyl]amino}-5,6,7,8-tetrahydro-7-quinolinyl)carbamate (1.64 g, 4.25 mmol)(actual weight 2.28 g as contains ethanol, 30% w/w) in anhydrous toluene (100 ml) was stirred at room temperature under argon and treated with ethyl oxoacetate (1.0 ml, 5.04 mmol)(50% solution in toluene) dropwise over 5 minutes. After 30 minutes, dried 3 A molecular sieves were added and the reaction was stirred at room temperature under argon for 4 hours. Ethyl oxoacetate (0.4 ml)(50% in toluene) was added and the orange reaction mixture was stirred at room temperature, under argon for 18 hours. Ethyl oxoacetate (0.2 ml) was added and the orange/brown mixture was stirred for 4 hours. Methanol (100 ml) was added and the solution was cooled in an ice-bath. Sodium borohydride (0.177 g, 4.68 mmol) was added in one portion and the reaction was stirred for 30 minutes in the cooling bath. The cooling bath was removed and the reaction was stirred for 30 minutes, filtered to remove the sieves which were washed well with 1:1 dichloromethane:methanol (200 ml). The very dark brown filtrate was treated with water (25 ml) and evaporated to remove the methanol and dichloromethane. The residue was treated with ethyl acetate (100 ml) and saturated aqueous brine (75 ml). The layers were separated and the aqueous layer was washed with ethyl acetate (100 ml). The combined organic extracts were dried over anhydrous sodium sulphate, filtered and evaporated to a black oil which was chromatographed eluting with a 0 to 100% ethyl acetate in iso-hexane then 0 to 20% methanol in ethyl acetate. The product fractions were evaporated to dryness to give an orange gum, 0.96 g, 48%.

MS (ES+) m/z 472.

### (f) 1,1-Dimethylethyl {3-[7-(methyloxy)-2-oxo-3,4-dihydropyrido[2,3-b]pyrazin-1(2H)-yl]-5,6,7,8-tetrahydro-7-quinolinyl}carbamate

A suspension of 60% sodium hydride (82 mg, 2.061 mmol) in mineral oil in anhydrous tetrahydrofuran (40 ml) was cooled to 0° C., under argon, and a solution of ethyl N-[3-{[7-({[(1,1-dimethylethyl)oxy]carbonyl}amino)-5,6,7,8-tetrahydro-3-quinolinyl]amino}-5-(methyloxy)-2-pyridinyl]glycinate (810 mg, 1.718 mmol) in anhydrous tetrahydrofuran (40 ml) was added drop wise over 10 minutes. Initially the reaction mixture went a green-blue colour but eventually the reaction mixture went a dark brown colour. 60% Sodium hydride (30 mg, 0.750 mmol) in mineral oil was added and the cooling bath was removed and the dark reaction was stirred for 20 minutes and then cooled in the ice bath for 5 minutes and then treated with saturated aqueous ammonium chloride (2 ml). The cooling bath was removed and the reaction was stirred for 10 minutes, diluted with ethyl acetate (100 ml) and treated with anhydrous sodium sulphate and stirred for 10 minutes. The suspension was filtered and evaporated to give the title compound as a brown solid, (900 mg, 123%) which was immediately reacted without purification.

MS (ES+) m/z 426 (MH⁺)

### (g) 1,1-Dimethylethyl {3-[7-(methyloxy)-2-oxopyrido[2,3-b]pyrazin-1(2H)-yl]-5,6,7,8-tetrahydro-7-quinolinyl}carbamate

A brown solution of 1,1-dimethylethyl {3-[7-(methyloxy)-2-oxo-3,4-dihydropyrido[2,3-b]pyrazin-1(2H)-yl]-5,6,7,8-tetrahydro-7-quinolinyl}carbamate (900 mg) in anhydrous dichloromethane (10 ml) was treated with activated manganese dioxide (1.5 g, 17.25 mmol) and the resulting suspension was stirred at room temperature for 3 hours, filtered through Kieselguhr and washed through with dichloromethane (250 ml). The dark brown filtrate was evaporated to dryness and chromatographed eluting with a 0 to 100% ethyl acetate in iso-hexane followed by a 0 to 20% methanol in ethyl acetate gradient. The product fractions were evaporated to dryness to a sticky orange glass (370 mg, 52%).

MS (ES+) m/z 424 (MH⁺).

### (h) 1-(7-Amino-5,6,7,8-tetrahydro-3-quinolinyl)-7-(methyloxy)pyrido[2,3-b]pyrazin-2(1H)-one hydrochloride

A solution of 1,1-dimethylethyl {3-[7-(methyloxy)-2-oxopyrido[2,3-b]pyrazin-1(2H)-yl]-5,6,7,8-tetrahydro-7-quinolinyl}carbamate (350 mg, 0.827 mmol) in anhydrous methanol (20 ml) and anhydrous dichloromethane (20 ml) was stirred at room temperature under argon and treated with 4M HCl in 1,4-dioxane (1.0 ml) and the resulting brown solution was stirred under argon for 1 hour and allowed to stand at room temperature, under argon, for 17 hours. 4M HCl in 1,4-dioxane (2 ml) was added and the reaction was stirred at room temperature, under argon for 5 hours. 4M HCl in 1,4-dioxane (2 ml) was added and the reaction was stirred at room temperature, under argon for 3 hours and then the dark orange reaction mixture was evaporated to dryness. The red oily residue was mixed with anhydrous methanol and anhydrous ether and evaporated to give a brown solid, 350 mg, 118%.

MS (ES+) m/z 324 (MH⁺).

### (i) Title Compound

A suspension of 1-(7-amino-5,6,7,8-tetrahydro-3-quinolinyl)-7-(methyloxy)pyrido[2,3-b]pyrazin-2(1H)-one (100 mg, 0.278 mmol)(hydrochloride salt) in anhydrous dichloromethane (DCM) (4 ml) and anhydrous methanol (0.4 ml) was treated with triethylamine (0.077 ml, 0.556 mmol) and 6,7-dihydro[1,4]dioxino[2,3-c]pyridazine-3-carbaldehyde (for a preparation see Example 5(1)) (50.8 mg, 0.306 mmol) and the resulting suspension was stirred at 20° C., under argon, for 15 minutes. The orange solution was treated with sodium triacetoxyborohydride (177 mg, 0.834 mmol) and the dark orange suspension was stirred at room temperature, under argon for 19 hours. 6,7-dihydro[1,4]dioxino[2,3-c]pyridazine-3-carbaldehyde (20 mg, 0.120 mmol) and sodium triacetoxyborohydride (100 mg, 0.472 mmol) were added and the reaction was stirred for 2 hours. 6,7-dihydro[1,4]dioxino[2,3-c]pyridazine-3-carbaldehyde (10 mg, 0.060 mmol) and sodium triacetoxyborohydride (50 mg, 0.236 mmol) were added and the dark orange suspension was stirred at room temperature, under argon for 2 hours. The mixture was treated with saturated aqueous sodium bicarbonate solution (2 ml) and stirred for 10 minutes, diluted with dichloromethane (20 ml) and saturated aqueous sodium bicarbonate solution (5 ml) and the layers were separated. The aqueous layer was washed with dichloromethane (2×20 ml). The organic layers were combined and dried by passing through a hydrophobic frit and evaporated to a yellow gum which was purified on a 20 g silica column eluted with a 0 to 20% methanol in dichloromethane. The product fractions were combined and evaporated to dryness. The residue was dissolved in dichloromethane and diluted with diethyl ether and the precipitate was collected by filtration to give the title compound as a yellow solid (20.8 mg, 16%).

MS (ES+) m/z 474 (MH⁺).

NMR (CDCl₃) 1.80 (1H, m), 2.16 (1H, m), 2.82-3.06 (3H, m), 3.20-3.38 (2H, m), 3.83 (3H, m), 4.13, (2H, m), 4.38 (2H, m), 4.53 (2H, m), 6.47-6.54 (1H, m), 7.0-7.07 (1H, m), 7.38 (1H, m), 8.33 (1H, m), 8.37 (1H, m), 8.44 (1H, m).

### Example 33

### 6-[({3-[7-(Methyloxy)-2-oxopyrido[2,3-b]pyrazin-1(2H)-yl]-5,6,7,8-tetrahydro-7-quinolinyl}amino)methyl]-2H-pyrido[3,2-b][1,4]oxazin-3(4H)-one

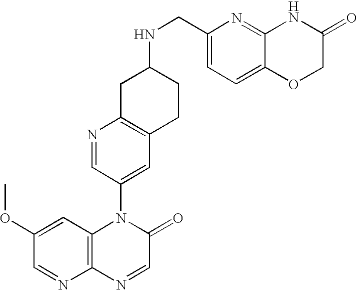

A suspension of 1-(7-amino-5,6,7,8-tetrahydro-3-quinolinyl)-7-(methyloxy)pyrido[2,3-b]pyrazin-2(1H)-one (100 mg, 0.278 mmol)(hydrochloride salt) (for a preparation see Example 32(h)) in anhydrous DCM (4 ml) and anhydrous methanol (0.4 ml) was treated with triethylamine (0.077 ml, 0.556 mmol) and 3-oxo-3,4-dihydro-2H-pyrido[3,2-b][1,4]oxazine-6-carbaldehyde (for a synthesis see WO03087098 Example 31(e)) (49.5 mg, 0.278 mmol) and the resulting suspension was stirred at 20° C., under argon, for 15 minutes. The orange solution was treated with sodium triacetoxyborohydride (177 mg, 0.834 mmol) and the yellow suspension was stirred for 18 hours, treated with aqueous saturated sodium bicarbonate solution (2 ml) and stirred for 10 minutes. The layers were separated and the aqueous layer was washed with 10:1 dichloromethane: methanol (2×10 ml). The organic extracts were combined and dried by passing through a hydrophobic frit and evaporated to a yellow gum. This gum was chromatographed eluting with a 0 to 20% methanol in dichloromethane gradient. The product fractions were combined and evaporated to a yellow gum. This gum was mixed with dichloromethane (5 ml) and diluted with diethyl ether (50 ml) and the pale yellow solid was collected by filtration and dried. This solid was dissolved in dichloromethane (25 ml) and washed with saturated aqueous sodium bicarbonate solution (2 ml). The layers were separated and the aqueous was washed with dichloromethane (25 ml). The combined organic layers were passed through a hydrophobic frit and evaporated to near dryness and treated with diethyl ether (50 ml) and the pale yellow solid was collected by filtration, washed with ether and dried to give the title compound as a pale yellow solid (33 mg, 23%).

MS (ES+) m/z 486 (MH⁺).

NMR (CDCl₃) 1.80-1.90 (1H, m), 2.10-2.22 (1H, m), 2.90-3.10 (3H, m), 3.12-3.25 (1H, m), 3.32-3.40 (1H, m), 3.83 (3H, m), 3.93 (2H, s) 4.65 (2H, d), 6.52 (1H, m), 6.97 (1H, m), 7.23 (1H, m), 7.39 (1H, m), 8.35 (2H, m), 8.44 (1H, m).

### Example 34

### 1-{7-[(2,3-Dihydro[1,4]dioxino[2,3-c]pyridin-7-ylmethyl)amino]-5,6,7,8-tetrahydro-3-quinolinyl}-7-(methyloxy)pyrido[2,3-b]pyrazin-2(1H)-one

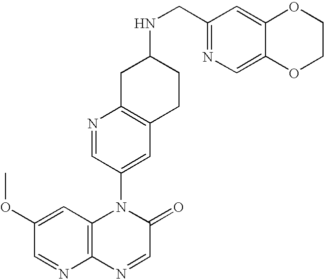

A solution of 1-(7-amino-5,6,7,8-tetrahydro-3-quinolinyl)-7-(methyloxy)pyrido[2,3-b]pyrazin-2(1H)-one (obtainable from the hydrochloride salt (for a preparation see Example 32(h)) by dissolving in methanol and purification on a SCX cartridge eluting with methanol, 0.2M ammonia in methanol, then 1M ammonia in methanol) (60 mg, 0.186 mmol) in anhydrous dichloromethane (DCM) (4 ml) and anhydrous methanol (0.4 ml) was treated with 2,3-dihydro[1,4]dioxino[2,3-c]pyridine-7-carbaldehyde (for synthesis see WO2004058144 Example 2(c) or WO2003087098 Example 19(d)) (30.6 mg, 0.186 mmol) and the solution was stirred at 20° C., under argon for 15 minutes. The orange solution was cooled in an ice-bath and sodium triacetoxyborohydride (118 mg, 0.557 mmol) was added in one go and the yellow solution was stirred at room temperature for 1.5 hours. The reaction mixture was treated with saturated aqueous sodium bicarbonate solution (1 ml) and stirred for 10 minutes. The layers were separated and the aqueous was washed with 10:1 DCM: methanol (2×20 ml). The organic extracts were combined and dried by passing through a hydrophobic frit and evaporated to a yellow gum which was chromatographed eluting with a 0 to 100% ethyl acetate in iso-hexane gradient followed by a 0 to 30% methanol in ethyl acetate gradient. The product fractions were combined and evaporated to a yellow gum which was mixed with dichloromethane (2 ml) and diluted with anhydrous diethyl ether (50 ml) and allowed to stand. The pale yellow solid was collected by filtration, washed well with ether and dried at 40° C. to give the title compound as a yellow solid (39.1 mg, 44%).

MS (ES+) m/z 473 (MH⁺).

NMR (CDCl₃) 1.80 (1H, m), 2.16 (1H, m), 2.82-3.06 (3H, m), 3.20-3.38 (2H, m), 3.83 (3H, m), 3.93 (2H, m), 4.30 (4H, m), 6.48-6.55 (1H, m), 6.82-6.87 (1H, m), 7.38 (1H, m), 8.14 (1H, m), 8.32-8.36 (2H, m), 8.44 (1H, m).

### Example 35

### 4-{7-[(2,3-Dihydro-1,4-benzodioxin-6-ylmethyl)amino]-5,6,7,8-tetrahydro-3-quinolinyl}-6-(methyloxy)pyrido[2,3-b]pyrazin-3(4H)-one hydrochloride

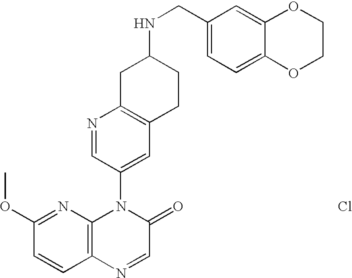

A solution of 4-(7-amino-5,6,7,8-tetrahydro-3-quinolinyl)-6-(methyloxy)pyrido[2,3-b]pyrazin-3(4H)-one (for a preparation see Example 6(f)) (55 mg, 0.170 mmol) in anhydrous dichloromethane (DCM) (2 ml) and anhydrous methanol (0.2 ml) was treated with 2,3-dihydro-1,4-benzodioxin-6-carbaldehyde (28 mg, 0.171 mmol) and the solution was stirred at 20° C., under argon for 15 minutes. The orange solution was cooled in an ice-bath and sodium triacetoxyborohydride (108 mg, 0.510 mmol) was added in one portion and the pale orange suspension was stirred in the cooling bath for 10 minutes and then the cooling bath was removed and the orange solution was stirred, under argon for 3 hours. Sodium triacetoxyborohydride (100 mg, 0.472 mmol) was added and the reaction was stirred for 2 hours 15 min and then treated with saturated aqueous sodium bicarbonate solution (1 ml) and stirred for 10 minutes. The reaction was diluted with dichloromethane (5 ml) and the layers were separated. The aqueous layer was washed with dichloromethane (10 ml) and the organic extracts were combined, dried by passing through a hydrophobic frit and evaporated to a yellow gum, which was chromatographed eluting with a 0 to 100% ethyl acetate in iso-hexane gradient, then a 0 to 30% methanol in ethyl acetate gradient. The product fractions were combined and evaporated to dryness to give the free base of the title compound as a cream solid (53 mg).

MS (ES+) m/z 472 (MH⁺).

NMR (CDCl₃)(400 Hz) 1.73-1.91 (1H, m), 2.10-2.23 (1H, m), 2.40-3.0 (3H, m), 3.12-3.22 (1H, m), 3.40-3.48 (1H, m), 3.67 (3H, s), 3.85 (2H, s), 4.25 (4H, s), 6.74 (1H, d), 6.84 (2H, m), 6.89 (1H, s), 7.42 (1H, d), 8.06 (1H, d), 8.27 (1H, s), 8.38 (1H, d).

This material was dissolved in 1:1 DCM:MeOH (2 ml) with 1M HCl in ether (3 ml) and a bright yellow precipitate formed. The suspension was diluted with anhydrous diethyl ether (10 ml) and the precipitate was collected by filtration, washed well with anhydrous ether (10 ml) and dried at 40° C. to give the title compound as cream solid (58 mg).

### Example 36

### 2-[({3-[6-(Methyloxy)-3-oxopyrido[2,3-b]pyrazin-4(3H)-yl]-5,6,7,8-tetrahydro-7-quinolinyl}amino)methyl]-1H-pyrimido[5,4-b][1,4]oxazin-7(6H)-one hydrochloride

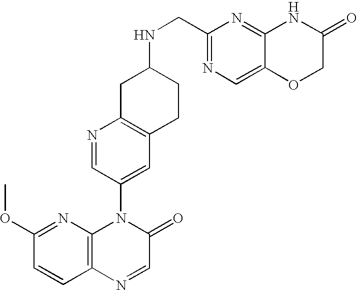

A solution of 4-(7-amino-5,6,7,8-tetrahydro-3-quinolinyl)-6-(methyloxy)pyrido[2,3-b]pyrazin-3(4H)-one (for a preparation see Example 6(f)) (103 mg, 0.32 mmol) in anhydrous dichloromethane (DCM) (4 ml) and anhydrous methanol (0.4 ml) was treated with 7-oxo-6,7-dihydro-1H-pyrimido[5,4-b][1,4]oxazine-2-carbaldehyde (for a synthesis see Preparation B) (51.6 mg, 0.288 mmol) and the solution was stirred at 20° C., under argon for 15 minutes. The orange solution was cooled in an ice-bath and sodium triacetoxyborohydride (203 mg, 0.960 mmol) was added in one go and the pale orange suspension was stirred for 10 minutes. The cooling bath was removed and the orange solution was stirred under argon for 5 hours. 7-oxo-6,7-dihydro-1H-pyrimido[5,4-b][1,4]oxazine-2-carbaldehyde (32 mg) was added in dichloromethane (2 ml), methanol (0.2 ml) along with sodium triacetoxy borohydride (70 mg) and the resulting mixture was stirred at room temperature, under argon for 18 hours. The reaction was treated with saturated aqueous sodium bicarbonate solution (1 ml) and stirred for 10 minutes. The layers were separated and the aqueous layer was washed with 10:1 dichloromethane:methanol (2×25 ml). The organic extracts were combined and passed through a hydrophobic frit to give a yellow gum which was chromatographed eluting with a 0 to 100% ethyl acetate in iso-hexane gradient, then 0 to 30% methanol in ethyl acetate gradient. The product fractions were combined and evaporated to dryness to give the free base of the title compound as a cream solid (40.0 mg).

MS (ES+) m/z 487 (MH⁺)

NMR (CDCl₃) 1.85 (1H, m), 2.15 (1H, m), 2.81-3.04 (3H, m), 3.23 (1H, m), 3.41 (1H, m), 3.67 (3H, s), 4.13 (2H, m), 4.72 (2H, s), 6.72 (1H, d), 7.44 (1H, s), 8.08 (1H, d), 8.26 (2H, m), 8.40 (1H, s)

This material was dissolved in anhydrous dichloromethane (3 ml) and treated with 1M HCl in ether (2 ml). A bright yellow suspension formed which was evaporated to give the title compound as cream solid (38 mg).

MS (ES+) m/z 487 (MH⁺)

### Example 37

### 6-[({7-[6-(Methyloxy)-3-oxopyrido[2,3-b]pyrazin-4(3H)-yl]-3,4-dihydro-2H-pyrano[3,2-b]pyridin-3-yl}amino)methyl]-2H-pyrido[3,2-b][1,4]oxazin-3(4H)-one fumarate

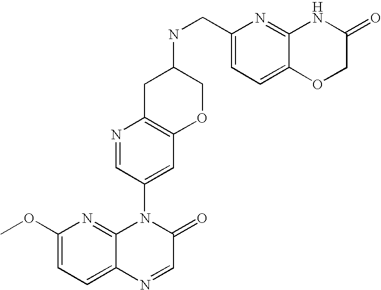

### (a) 6-(Methyloxy)-4-(3-{[(phenylmethyl)oxy]amino}-3,4-dihydro-2H-pyrano[3,2-b]pyridin-7-yl)pyrido[2,3-b]pyrazin-3(4H)-one

6-(Methyloxy)-4-(2H-pyrano[3,2-b]pyridin-7-yl)pyrido[2,3-b]pyrazin-3(4H)-one (for a preparation see Example 17(f) (450 mg, 1.460 mmol) and O-benzylhydroxylamine HCl (0.466 g, 2.92 mmol) in methanol (10 ml) were heated under reflux overnight. The solvent was evaporated under vacuum, the residue was dissolved in dichloromethane and aqueous sodium carbonate and the phases were separated. The aqueous phase was extracted with dichloromethane three times, the organic fractions were dried over sodium sulphate and evaporated under vacuum. The crude product was chromatographed on 50 g silica eluted with 50-100% ethyl acetate/isohexane to give the product (0.35 g, 56%).

MS (+ve ion electrospray) m/z 432 (MH+).

### (b) 4-(3-Amino-3,4-dihydro-2H-pyrano[3,2-b]pyridin-7-yl)-6-(methyloxy)-1,4-dihydropyrido[2,3-b]pyrazin-3(2H)-one

6-(Methyloxy)-4-(3-{[(phenylmethyl)oxy]amino}-3,4-dihydro-2H-pyrano[3,2-b]pyridin-7-yl)pyrido[2,3-b]pyrazin-3(4H)-one (0.39 g, 0.904 mmol) was dissolved in methanol (30 ml), filtered, and hydrogenated using a flow reactor with a 10% palladium/carbon catalyst cartridge (flow rate 1 ml/min, 25° C., 40 bar pressure). The eluted product solution was concentrated to its original volume and passed through the apparatus again with a fresh catalyst cartridge. The mixture was evaporated and the residue was chromatographed on 10 g silica eluted with 0-20% methanol/dichloromethane to give the required product (eluted second, 66 mg, 22%).

MS (+ve ion electrospray) m/z 328 (MH+).

A partially reduced product, 6-(methyloxy)-4-(3-{[(phenylmethyl)oxy]amino}-3,4-dihydro-2H-pyrano[3,2-b]pyridin-7-yl)-1,4-dihydropyrido[2,3-b]pyrazin-3(2H)-one, was also obtained (0.183 g, 47%).

MS (+ve ion electrospray) m/z 434 (MH+).

### (c) 4-(3-Amino-3,4-dihydro-2H-pyrano[3,2-b]pyridin-7-yl)-6-(methyloxy)pyrido[2,3-b]pyrazin-3(4H)-one

4-(3-Amino-3,4-dihydro-2H-pyrano[3,2-b]pyridin-7-yl)-6-(methyloxy)-1,4-dihydropyrido[2,3-b]pyrazin-3(2H)-one (66 mg, 0.202 mmol) was stirred in dichloromethane (5 ml)/methanol (0.5 ml) with manganese(IV) oxide (0.35 g, 4.03 mmol) at room temperature for 2.5 h, then the mixture was filtered through Kieselguhr, washing through thoroughly with 10% methanol/dichloromethane. The liquor was evaporated to give the product (56 mg, 85%).

MS (+ve ion electrospray) m/z 326 (MH+).

### (d) Title Compound

4-(3-Amino-3,4-dihydro-2H-pyrano[3,2-b]pyridin-7-yl)-6-(methyloxy)pyrido[2,3-b]pyrazin-3(4H)-one (56 mg, 0.172 mmol) and 3-oxo-3,4-dihydro-2H-pyrido[3,2-b][1,4]oxazine-6-carbaldehyde (for a synthesis see WO03087098 Example 31(e))(30.7 mg, 0.172 mmol) were stirred together with 3 A molecular sieves in dry chloroform (5 ml) for 7.5 h. Sodium triacetoxyborohydride (182 mg, 0.861 mmol) was added and the mixture was stirred at room temperature overnight. The mixture was washed with aqueous sodium bicarbonate and the phases were separated. The aqueous phase was extracted with 10% methanol/dichloromethane three times, the organic fractions were dried over sodium sulphate and evaporated under vacuum. The crude product was chromatographed on 5 g silica eluted with 0-20% methanol/dichloromethane to give the free base of the title compound (67 mg, 80%).

¹H NMR (CDCl₃) δ 8.48 (1H, broad), 8.27 (1H, s), 8.27 (1H, d), 8.08 (1H, d), 7.23 (1H, d), 7.17 (1H, d), 6.97 (1H, s), 6.74 (1H, d), 4.65 (2H, s), 4.30 (1H, d), 4.06 (1H, m), 3.96 (2H, s), 3.68 (3H, s), 3.36 (1H, m), 3.29 (1H, dd), 2.70 (2H, m), 2.98 (1H, dd).

MS (+ve ion electrospray) m/z 488 (MH+).

The free base of the title compound was dissolved in dichloromethane (2 ml) and treated with a solution of fumaric acid in methanol (2 ml). The solution was evaporated and the residue was dried under high vacuum to give the title fumarate salt (77 mg).

### Preparation A

### 7-Oxo-5,6,7,8-tetrahydropyrido[2,3-d]pyrimidine-2-carbaldehyde

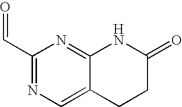

### (a) 3-Ethyl 1,1-dimethyl 1,1,3-propanetricarboxylate

To a solution of dimethyl malonate (2.5 g, 18.9 mmol) in anhydrous THF (20 mL) was added NaH (0.038 g, 0.95 mmol, 60% in mineral oil). The reaction was stirred at ambient temperature for 15 minutes. In a separate flask, ethyl acrylate was dissolved in anhydrous THF (Imp and then added dropwise over 30 minutes to the dimethyl malonate solution. The reaction was stirred at ambient temperature for 16 h and then concentrated under vacuum. The residue was dissolved in EtOAc (ethyl acetate) and washed with saturated NH₄Cl solution and brine. The organic phase was dried over Na₂SO₄, filtered, and concentrated under vacuum. The crude residue was purified by column chromatography (silica gel) using an EtOAc/hexanes gradient to yield the desired compound as a colorless oil (1.68 g, 77%).

¹H NMR (400 MHz, CDCl₃) δ 1.24 (t, J=7.07 Hz, 3H) 2.20 (q, J=7.24 Hz, 2H) 2.37 (t, J=7.33 Hz, 2H) 3.47 (t, J=7.33 Hz, 1H) 3.70-3.75 (m, 6H) 4.12 (q, J=7.24 Hz, 2H).

### (b) (2E)-3-Phenyl-2-propenimidamide

Cinnamonitrile (25.0 g, 194 mmol) was dissolved in EtOH (ethanol) (200 mL). The solution was cooled to 0° C. and HCl gas bubbled through the solution for 30 minutes. The solution was stirred at ambient temperature for 16 h and then concentrated under vacuum. The residue was dissolved in EtOH (100 mL), cooled to 0° C. and a solution of NH₃/MeOH (7M, 69 mL, 484 mmol) was added dropwise through an addition funnel. Once added, the solution was allowed to warm to ambient temperature and the resulting NH₄Cl was filtered off. The solution was concentrated under vacuum and the resulting white solid was used without further purification (26 g crude).

LCMS: m/z 147.4 (MH+).

### (c) Ethyl 3-{4-hydroxy-6-oxo-2-[(E)-2-phenylethenyl]-1,6-dihydro-5-pyrimidinyl}propanoate

3-Ethyl 1,1-dimethyl 1,1,3-propanetricarboxylate (1.65 g, 7.11 mmol) and (2E)-3-phenyl-2-propenimidamide (1.04 g, 7.11 mmol) were combined in EtOH (36 mL). Triethylamine (1.98 mL, 14.2 mmol) was added and the solution was heated at reflux for 3 h with no change based on LCMS. The solution was cooled to room temperature and treated with NaOMe in MeOH (1.0 mL, 5.33 mmol, 25% wt solution) and the solution was refluxed for an additional 4 h. Another portion of NaOMe in MeOH (1.0 mL, 5.33 mmol, 25% wt solution) was added and the solution was refluxed for 16 h. After this time, a yellow precipitate had formed which was filtered off. The mother liquor was acidified to pH2 with 1N HCl, and the solution was concentrated under vacuum. The resulting material was combined with the yellow solid and used without further purification.

LCMS: m/z 315.2 (MH+).

### (d) Ethyl 3-{4,6-dichloro-2-[(E)-2-phenylethenyl]-5-pyrimidinyl}propanoate

Crude ethyl 3-{4-hydroxy-6-oxo-2-[(E)-2-phenylethenyl]-1,6-dihydro-5-pyrimidinyl}propanoate was dissolved in POCl₃ (25 mL) and N,N-dimethylaniline (0.9 mL, 7.1 mmol) was slowly added to the solution. The reaction was then heated at reflux for 2 h. After cooling to ambient temperature, the resulting solution was carefully and slowly added to ice water to quench the excess POCl₃. The mixture was extracted with EtOAc (3×), dried over Na₂SO₄, filtered and concentrated under vacuum. The crude residue was then purified by column chromatography (silica gel) using an EtOAc/hexanes gradient to yield the desired compound as a yellow solid (0.48 g, 19% over 2 steps).

LCMS: m/z 351.4 (MH+).

### (e) 4-Chloro-2-[(E)-2-phenylethenyl]-5,8-dihydropyrido[2,3-d]pyrimidin-7(6H)-one

To a solution of ethyl 3-{4,6-dichloro-2-[(E)-2-phenylethenyl]-5-pyrimidinyl}propanoate (0.42 g, 1.19 mmol) in 1,4-dioxane (5 mL) was added conc. NH₄OH (3.5 mL). The reaction was heated at 75° C. in a sealed tube for 16 h. The solution was concentrated under vacuum, diluted with water, and extracted with EtOAc. The organic layer was washed with brine, dried over Na₂SO₄, and concentrated under vacuum. The crude residue was then purified by column chromatography (silica gel) to yield the desired compound as a yellow solid (0.072 g, 21%).

LCMS: m/z 286.2 (MH+).

Also obtained was 3-{4-amino-6-chloro-2-[(E)-2-phenylethenyl]-5-pyrimidinyl}propanamide as a white solid (0.175 g).

LCMS: m/z 303.3 (MH+).

3-{4-Amino-6-chloro-2-[(E)-2-phenylethenyl]-5-pyrimidinyl}propanamide (0.175 g, 0.58 mmol) was dissolved in EtOH (15 mL) and HCl gas was bubbled through the solution until saturated. The solution was heated at reflux for 2 h, cooled to ambient temperature and concentrated under vacuum. The residue was dissolved in water and extracted with EtOAc (3×. The organic layers were combined, dried over Na₂SO₄, filtered and concentrated under vacuum to yield ethyl 3-{4-amino-6-chloro-2-[(E)-2-phenylethenyl]-5-pyrimidinyl}propanoate as a white solid. LCMS: m/z 332.2 (MH⁺). This product was then dissolved in DMF (5 mL), treated with K₂CO₃ (0.16 g, 1.16 mmol) and heated at 75° C. for 30 minutes. The solution was cooled, diluted with water and extracted with Et₂O (3×). The organic layer was dried over Na₂SO₄, filtered, and concentrated under vacuum. The crude residue was then purified by column chromatography (silica gel) to yield an additional 0.11 g of the desired compound as an off-white solid.

LCMS: m/z 286.2 (MH⁺).

### (f) 4-Chloro-7-oxo-5,6,7,8-tetrahydropyrido[2,3-d]pyrimidine-2-carbaldehyde

4-Chloro-2-[(E)-2-phenylethenyl]-5,8-dihydropyrido[2,3-d]pyrimidin-7(6H)-one (0.18 g, 0.64 mmol) was dissolved in a 2:1 solution of 1,4-dioxane/water (6 mL) and cooled to 0° C. NaIO₄ (0.314 g, 1.47 mmol) and catalytic OsO₄ (1 mL, 4% aq. solution) were added and the solution was then stirred at ambient temperature for 16 h. The reaction solution was concentrated under vacuum, diluted with water, and extracted with 10% MeOH/DCM (4×). The organic layers were combined, dried over Na₂SO₄, filtered and concentrated under vacuum. The crude residue was then purified by column chromatography (silica gel) using a DCM/DCM-MeOH—NH₄OH (90:10:1) gradient to yield the desired compound as an off-white solid (0.05 g, 44%).

LCMS: m/z 212.0 (MH+).

### (g) 2-[Bis(methyloxy)methyl]-4-chloro-5,8-dihydropyrido[2,3-d]pyrimidin-7(6H)-one

To a solution of 4-chloro-7-oxo-5,6,7,8-tetrahydropyrido[2,3-d]pyrimidine-2-carbaldehyde (1.43 g, 6.78 mmol) in MeOH (50 mL) was added p-toluenesulfonic acid monohydrate (p-TsOH.H₂O) (0.13 g, 0.68 mmol). The solution was heated at reflux for 3 h and then cooled to ambient temperature. The solution was concentrated under vacuum to yield the desired product as a white solid which was used without further purification.

LCMS: m/z 257.9 (MH+).

### (h) 2-[Bis(methyloxy)methyl]-5,8-dihydropyrido[2,3-d]pyrimidin-7(6H)-one

To crude 2-[bis(methyloxy)methyl]-4-chloro-5,8-dihydropyrido[2,3-d]pyrimidin-7(6H)-one (prepared from 1.43 g 4-chloro-7-oxo-5,6,7,8-tetrahydropyrido[2,3-d]pyrimidine-2-carbaldehyde) dissolved in MeOH (50 mL) was added 10% Pd/C (wet) (0.15 g). The solution was stirred under an atmosphere of H₂ (balloon) overnight. The Pd/C was filtered off and the solution concentrated under vacuum. The crude residue was purified by column chromatography (silica gel) using a DCM/DCM-MeOH—NH₄OH (90:10:1) gradient to yield the desired product as a white solid (0.873 g, 58% over 2 steps).

LCMS: m/z 223.9 (MH+).

### (i) Title Compound

To a solution of 2-[bis(methyloxy)methyl]-5,8-dihydropyrido[2,3-d]pyrimidin-7(6H)-one (0.87 g, 3.91 mmol) in 1:1 H₂O/acetone (10 mL) was added p-TsOH.H₂O (0.074 g, 0.39 mmol) and the reaction was heated at reflux for 2 days. The reaction was not complete, so additional p-TsOH.H₂O (0.20 g) was added and the solution was refluxed for an additional 1 day. After the disappearance of starting material, the solution was concentrated under vacuum to yield the desired product as a white solid and the crude material was used directly in the next step (1.023 g).

LCMS: m/z 178.0 (MH+).

### Preparation B

### 7-Oxo-6,7-dihydro-1H-pyrimido[5,4-b][1,4]oxazine-2-carbaldehyde

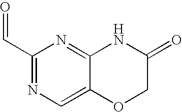

### (a) 2-[(E)-2-Phenylethenyl]-5-(tetrahydro-2H-pyran-2-yloxy)-4(1H)-pyrimidinone

NaH (0.38 g, 9.5 mmol, 60% paraffin oil) was added slowly to a THF (20 mL) solution of ethyl (tetrahydro-2H-pyran-2-yloxy)acetate (prepared by treating ethyl hydroxyacetate with 3,4-dihydro-2H-pyran and p-toluenesulphonic acid) (1.0 g, 5.3 mmol) and dry ethyl formate (3.9 g, 53 mmol). The reaction mixture was stirred at room temperature for 15 min. and then heated at 65° C. for 45 min. The reaction mixture was concentrated to dryness to give a pale yellow solid. The solid was added to a MeOH/EtOH (20 mL/20 mL) solution of (2E)-3-phenyl-2-propenimidamide (for a synthesis see Preparation A(b)) (0.78 g, 5.3 mmol), the subsequent mixture was heated at 80° C. for 4 h. The resulting material was poured into DCM (10 mL) containing silica gel (3 g) and evaporated. Purification by column chromatography (silica gel) using a MeOH/DCM gradient (0-10%) provided the desired product as a pale yellow solid (1 g, 63%).

LCMS: m/z 299 (MH⁺).

### (b) 2-[(E)-2-Phenylethenyl]-5-(tetrahydro-2H-pyran-2-yloxy)-4-pyrimidinyl trifluoromethanesulfonate

To a suspension of 2-[(E)-2-phenylethenyl]-5-(tetrahydro-2H-pyran-2-yloxy)-4(1H)-pyrimidinone (2.04 g, 6.84 mmol) in DCM (25 mL) was added pyridine (1.22 mL, 15.05 mmol). After cooling to −78° C., trifluoromethanesulphonic anhydride (1.38 mL, 8.2 mmol) was slowly added via dropwise addition. The reaction was maintained at −78° C. for 10 minutes, after which time the cooling bath was replaced with a ice-water bath and the reaction was stirred for an additional 0.5 h. The reaction mixture was poured into water and the aqueous phase was extracted with DCM. The organic phase was then washed with water, saturated aq. NaHCO₃, and brine. The organic phase was dried over Na₂SO₄, filtered, and concentrated under vacuum to provide a dark reddish oil which was used directly in the next step.

LCMS: m/z 431.0 (MH⁺).

### (c) 2-[(E)-2-Phenylethenyl]-5-(tetrahydro-2H-pyran-2-yloxy)-4-pyrimidinamine

Crude 2-[(E)-2-phenylethenyl]-5-(tetrahydro-2H-pyran-2-yloxy)-4-pyrimidinyl trifluoromethanesulfonate (6.8 mmol) was reacted with a 0.5M solution of ammonia in 1,4-dioxane (136 mL) in a pressure bottle at 60° C. for 24 h. The reaction was concentrated under vacuum, the residue was taken up in DCM and washed with water, saturated aq. NaHCO₃ and brine. The organic phase was dried over Na₂SO₄, filtered and concentrated. The crude residue was purified by column chromatography (silica gel) using a MeOH/DCM gradient to yield the desired compound as a tan solid (1.28 g, 63% for two steps).

LCMS: m/z 298.0 (MH+).

### (d) 4-Amino-2-[(E)-2-phenylethenyl]-5-pyrimidinol, hydrochloride

2-[(E)-2-Phenylethenyl]-5-(tetrahydro-2H-pyran-2-yloxy)-4-pyrimidinamine (1.28 g, 4.3 mmol) was suspended in MeOH (25 mL) and heated in a 50° C. oil bath until fully dissolved. To this was added 4M HCl in 1,4-dioxane (0.11 mL, 0.43 mmol) and the reaction was heated at 50° C. for 1.5 h. At this time, LCMS indicated little progression, therefore an additional 1.1 mL of 4M HCl/1,4-dioxane was added and heating was continued for 3 h. The reaction was allowed to cool to room temperature resulting in the formation of a white precipitate. The solvent was removed under vacuum and the resulting tan solid was dried under high vacuum over night yielding 1.08 g (100%, for HCl salt). This material was used without further purification.

LCMS: m/z 214.0 (MH+).

### (e) 2-[(E)-2-Phenylethenyl]-1H-pyrimido[5,4-b][1,4]oxazin-7(6H)-one

To a suspension of 4-amino-2-[(E)-2-phenylethenyl]-5-pyrimidinol hydrochloride (250 mg, 1.0 mmol) in absolute ethanol (5 mL) was added potassium tert-butoxide (224 mg, 2.0 mmol) at room temperature. After stirring for 5 minutes, ethyl bromoacetate (0.11 mL, 1.0 mmol) was added via dropwise addition and the reaction was stirred for 18 h. The solvent was evaporated and the residue was taken up in 10% MeOH—CHCl₃ and a small amount of water. The layers were separated and the aqueous phase was extracted with 10% MeOH—CHCl₃ (3×). The combined organic extracts were concentrated and the resulting solid was triterated with EtOAc. The white solid was collected by filtration (106 mg, 42%).

LCMS: m/z 254.0 (MH+).

### (f) Title Compound

To a suspension of 2-[(E)-2-phenylethenyl]-1H-pyrimido[5,4-b][1,4]oxazin-7(6H)-one (106 mg, 0.418 mmol) in 1,4-dioxane (12 mL) and water (3 mL) was added NaIO₄ (357 mg, 1.67 mmol) and OsO₄ (0.1 mL, 4% wt in water) and the reaction mixture was stirred at room temperature. After 2 h, and additional 3 mL of 1,4-dioxane and 180 mg of NaIO₄ were added. After a total of 7.5 h, the reaction was capped and stored in a freezer for the weekend. After warming to room temperature, additional OsO₄ (0.1 mL, 4% wt in water) was added and the reaction was stirred for an additional 4 h. The solvent was evaporated to give a white solid which was dissolved in DCM and water. The aqueous layer was extracted with 10% MeOH-DCM (6×). The combined organic extracts were dried over Na₂SO₄, filtered, and concentrated to give a light tan solid (92 mg) which was not purified further.

LCMS: m/z 180.0 (MH+).

### Biological Activity

### Antimicrobial Activity Assay:

Whole-cell antimicrobial activity was determined by broth microdilution using the Clinical Laboratory Standards Institute (CLSI) recommended procedure, Document M7-A7, “Methods for Dilution Susceptibility Tests for Bacteria that Grow Aerobically”. The compounds were tested in serial two-fold dilutions ranging from 0.016 to 16 mcg/mL.

The minimum inhibitory concentration (MIC) was determined as the lowest concentration of compound that inhibited visible growth. A mirror reader was used to assist in determining the MIC endpoint.

Compounds were evaluated against Gram-positive organisms, including Staphylococcus aureus, Streptococcus pneumoniae, Streptococcus pyogenes, Enterococcus faecalis and Enterococcus faecium.

In addition, compounds were evaluated against Gram-negative organisms including Haemophilus influenzae, Moraxella catarrhalis, Escherichia coli, Pseudomonas aeruginosa, Proteus mirabilis, Enterobacter cloacae, Enterobacter aerogenes, Klebsiella pneumoniae, Acinetobacter baumanii and Stenotrophomonas maltophilia.

Each of the listed Examples, as identified in the present application, except Examples 15, were tested in at least one exemplified salt or free base form. Example 37 was tested against Gram-negative organisms only. Tested examples had a MIC≦2 μg/ml against at least one of the organisms listed above, with the exception of Example 17 which was active at 16 μg/ml against at least one of the organisms listed above. For at least one strain of every organism listed above, at least one Example had a MIC≦2 μg/ml.

Mycobacterium tuberculosis H37Rv Inhibition Assay

The measurement of the minimum inhibitory concentration (MIC) for each tested compound was performed in 96 wells flat-bottom, polystyrene microtiter plates. Ten two-fold drug dilutions in neat DMSO starting at 400 μM were performed. Five μl of these drug solutions were added to 95 μl of Middlebrook 7H9 medium. (Lines A-H, rows 1-10 of the plate layout). Isoniazid was used as a positive control, 8 two-fold dilution of Isoniazid starting at 160 μgml⁻¹ was prepared and 5 μl of this control curve was added to 95 μl of Middlebrook 7H9 (Difco catalogue Ref. 271310)+ADC medium (Becton Dickinson Catalogue Ref 211887). (Row 11, lines A-H). Five μl of neat DMSO were added to row 12 (growth and Blank controls).

The inoculum was standardised to approximately 1×10⁷ cfu/ml and diluted 1 in 100 in Middlebrook 7H9+ADC medium and 0.025% Tween 80 (Sigma P4780), to produce the final inoculum of H37Rv strain (ATCC25618). One hundred μl of this inoculum was added to the entire plate but G-12 and H-12 wells (Blank controls). All plates were placed in a sealed box to prevent drying out of the peripheral wells and they were incubated at 37° C. without shaking for six days. A resazurin solution was prepared by dissolving one tablet of resazurin (Resazurin Tablets for Milk Testing; Ref 330884Y VWR International Ltd) in 30 ml sterile PBS (phosphate buffered saline). 25 μl of this solution was added to each well. Fluorescence was measured (Spectramax M5 Molecular Devices, Excitation 530 nm, Emission 590 nm) after 48 hours to determine the MIC value.

Examples 6-14, 18 and 26-29 were tested in the Mycobacterium tuberculosis H37Rv inhibition assay. Examples 6-9, 12-14, 18 and 28 showed an MIC value of 1.1 μg/ml or lower. Examples 6, 9, 12 and 28 showed an MIC value of 0.2 μg/ml or lower.

1. A compound of formula (I) or a pharmaceutically acceptable salt or N-oxide thereof: 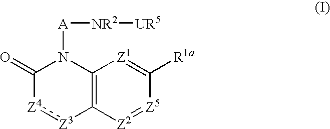
wherein:
Z⁴ is CH and two of Z¹, Z² and Z³ are independently CR^{1b} or N and the remainder are independently CR^{1b}, with a double bond between Z³ and Z⁴;
or one of Z¹ and Z² is CR^{1b} or N and the other is independently CR^{1b}, Z³ is O and Z⁴ is CH₂;
Z⁵ is CH or CF when Z² is CR^{1b}, or CH when Z² is N;
R^{1a} and R^{1b} are independently selected from hydrogen; halogen; cyano; nitro; (C₁₋₆)alkyl; (C₁₋₆)alkylthio; mono-, di- or tri-fluoromethyl; mono-, di- or tri-fluoromethoxy; carboxy; (C₁₋₆)alkoxycarbonyl; hydroxy optionally substituted with (C₁₋₆)alkyl or (C₁₋₆)alkoxy-substituted(C₁₋₆)alkyl; (C₁₋₆)alkoxy-substituted(C₁₋₆)alkyl; hydroxy (C₁₋₆)alkyl; an amino group optionally N-substituted by one or two (C₁₋₆)alkyl, (C₁₋₆)alkylcarbonyl or (C₁₋₆)alkylsulphonyl groups; and aminocarbonyl wherein the amino group is optionally substituted by one or two (C₁₋₄)alkyl; or R^{1a} and R^{1b} at Z¹ may together form an ethylenedioxy group;
or when one of Z² and Z³ is CR^{1b}, R^{1b} may instead be:
(C₃₋₆)cycloalkyl; (C₃₋₆)cycloalkoxy; (C₂₋₆)alkenyl optionally substituted by carboxy, (C₁₋₆)alkoxycarbonyl or aminocarbonyl wherein the amino group is optionally substituted by one or two (C₁₋₄)alkyl; (C₁₋₆)alkylcarbonyl; (C₁₋₆)alkylcarbonyl oxime; (C₁₋₄)alkyloxycarbonyl(C₁₋₆)alkyloxy; (C₁₋₄)alkylaminocarbonyl(C₁₋₆)alkyloxy; amino substituted by (C₁₋₄)alkylaminocarbonyl; aminocarbonyl wherein the amino group is substituted by (C₁₋₄)alkoxysulphonyl, hydroxy(C₁₋₄)alkyl, (C₁₋₄)alkoxy-substituted (C₁₋)alkyl, (C₃₋₆)cycloalkyl, phenyl, benzyl, monocyclic heteroaryl or monocyclic heteroaryl-methyl; benzyloxy; phenyl; benzyl; monocyclic heteroaryl; or monocyclic heteroaryl-methyl;
wherein heteroaryl is a 5 or 6 membered ring containing up to four hetero-atoms selected from oxygen, nitrogen and sulphur, and wherein a heteroaryl or phenyl ring in R^{1b} may be optionally C-substituted by up to three groups selected from (C₁₋₄)alkylthio; halo; carboxy(C₁₋₄)alkyl; halo(C₁₋₄)alkoxy; halo(C₁₋₄)alkyl; (C₁₋₄)alkyl; (C₂₋₄)alkenyl; (C₁₋₄)alkoxycarbonyl; formyl; (C₁₋₄)alkylcarbonyl; (C₂₋₄)alkenyloxycarbonyl; (C₂₋₄)alkenylcarbonyl; (C₁₋₄)alkylcarbonyloxy; (C₁₋₄)alkoxycarbonyl(C₁₋₄)alkyl; hydroxy; hydroxy(C₁₋₄)alkyl; mercapto(C₁₋₄)alkyl; (C₁₋₄)alkoxy; nitro; cyano; carboxy; amino or aminocarbonyl optionally substituted by one or two (C₁₋₄)alkyl; (C₁₋₄)alkylsulphonyl; (C₂₋₄)alkenylsulphonyl; or aminosulphonyl wherein the amino group is optionally substituted by (C₁₋₄)alkyl or (C₂₋₄)alkenyl;
R² is hydrogen, or (C₁₋₄)alkyl;
A is a group (i) or (ii): 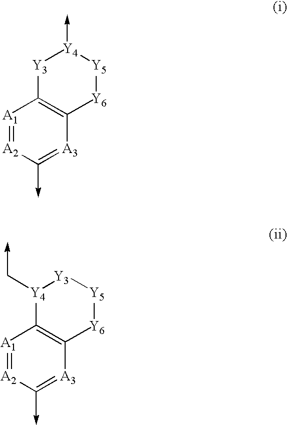
A₁, A₂ and A₃ are independently N or CR³; or
A₃ is N and A₁ and A₂ together form O, S, or NR⁴;
Y₃, Y₅ and Y₆ are independently CHR³, CO or X;
Y₄ is CR³;
X is NR⁴ or O;
provided that no more than one group Y₃, Y₅ and Y₆ is X and no more than one group Y₃, Y₅ and Y₆ is CO;
and provided that A is optionally substituted by up to two groups R³;
R³ is as defined for R^{1a} or is carboxy(C₁₋₄)alkyl or amino(C₁₋₄)alkyl where the amino group is optionally N-substituted by one or two (C₁₋₄)alkyl or (C₁₋₄)alkylcarbonyl groups;
R⁴ is hydrogen; methyl; carboxy(C₁₋₄)alkyl; (C₂₋₄)alkyl optionally substituted with hydroxy, (C₁₋₄)alkoxy or amino wherein the amino group is optionally substituted by one or two (C₁₋₄)alkyl, (C₁₋₄)alkoxycarbonyl (C₁₋₄)alkylcarbonyl or (C₁₋₄)alkylsulphonyl groups; wherein any alkyl group in R⁴ is optionally substituted with 1-3 fluorine atoms;
U is selected from CO, and CH₂ and
R⁵ is an optionally substituted bicyclic carbocyclic or heterocyclic ring system (B): 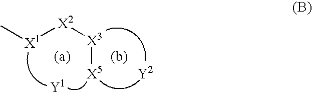
containing up to four heteroatoms in each ring in which
at least one of rings (a) and (b) is aromatic;
X¹ is C or N when part of an aromatic ring, or CR¹⁴ when part of a non-aromatic ring;
X² is N, NR¹³, O, S(O)ₓ, CO or CR¹⁴ when part of an aromatic or non-aromatic ring or may in addition be CR¹⁴R¹⁵ when part of a non aromatic ring;
X³ and X⁵ are independently N or C;
Y¹ is a 0 to 4 atom linker group each atom of which is independently selected from N, NR¹³, O, S(O)ₓ, CO and CR¹⁴ when part of an aromatic or non-aromatic ring or may additionally be CR¹⁴R¹⁵ when part of a non aromatic ring;
Y² is a 2 to 6 atom linker group, each atom of Y² being independently selected from N, NR¹³, O, S(O)ₓ, CO, CR¹⁴ when part of an aromatic or non-aromatic ring or may additionally be CR¹⁴R¹⁵ when part of a non aromatic ring;
each of R¹⁴ and R¹⁵ is independently selected from: H; (C₁₋₄)alkylthio; halo; carboxy(C₁₋₄)alkyl; (C₁₋₄)alkyl; (C₁₋₄)alkoxycarbonyl; (C₁₋₄)alkylcarbonyl; (C₁₋₄)alkoxy (C₁₋₄)alkyl; hydroxy; hydroxy(C₁₋₄)alkyl; (C₁₋₄)alkoxy; nitro; cyano; carboxy; amino or aminocarbonyl optionally mono- or di-substituted by (C₁₋₄)alkyl; or
R¹⁴ and R¹⁵ may together represent oxo;
each R¹³ is independently H; trifluoromethyl; (C₁₋₄)alkyl optionally substituted by hydroxy, (C₁₋₆)alkoxy, (C₁₋₆)alkylthio, halo or trifluoromethyl; (C₂₋₄)alkenyl; (C₁₋₄)alkoxycarbonyl; (C₁₋₄)alkylcarbonyl; (C₁₋₆)alkylsulphonyl; aminocarbonyl wherein the amino group is optionally mono or disubstituted by (C₁₋₄)alkyl;
each x is independently 0, 1 or 2.

2. A compound according to claim 1 selected from compounds of formulae (IA), (113) and (C):

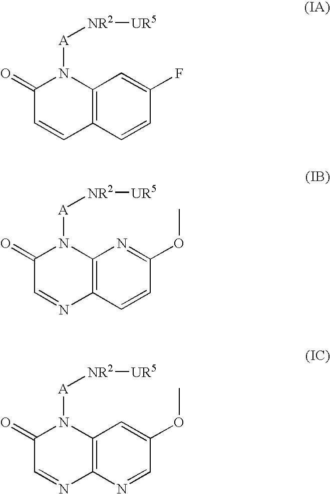

3. A compound according to claim 1 wherein A is a group selected from the group consisting of: 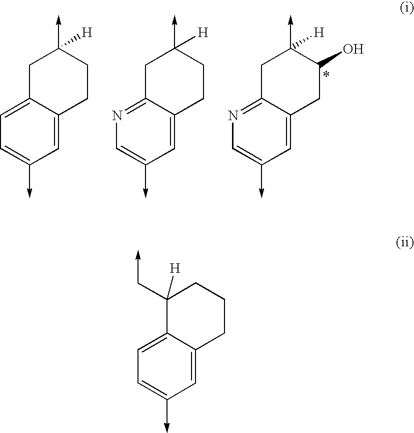
*relative stereochemistry, includes either or both cis diastereomers

4. A compound according to claim 1 wherein R² is hydrogen.

5. A compound according to claim 1 wherein U is CH₂.

6. A compound according to claim 1 wherein R⁵ is an aromatic heterocyclic ring (B) having 8-11 ring atoms including 2-4 heteroatoms of which at least one is N or NR¹³ in which Y² contains 2-3 heteroatoms, one of which is S and 1-2 are N, with one N bonded to X³, or the heterocyclic ring (B) has ring (a) aromatic selected from optionally substituted benzo, pyrido, pyridazino and pyrimidino and ring (b) non aromatic and Y² has 3-4 atoms including at least one heteroatom, with O, S, CH₂ or NR¹³ bonded to X⁵, where R¹³ is other than hydrogen, and either NHCO bonded via N to X³, or O, S, CH₂, or NH bonded to X³.

7. A compound according to claim 1 wherein R⁵ is selected from the group consisting of:
6-substituted 2H-pyrido[3,2-b][1,4]oxazin-3(4H)-one;
2,3-dihydro-[1,4]dioxino[2,3-c]pyridin-7-yl;
[1,3]oxathiolo[5,4-c]pyridin-6-yl;
3,4-dihydro-2H-pyrano[2,3-c]pyridine-6-yl;
6-substituted 2H-pyrido[3,2-b][1,4]thiazin-3(4H)-one;
6-substituted 7-chloro-2H-pyrido[3,2-b][1,4]oxazin-3(4H)-one; and
6,7-dihydro[1,4]dioxino[2,3-c]pyridazin-3-yl.

8. A compound according to claim 1 selected from the group consisting of:
1-{(6S)-6-[(2,3-Dihydro[1,4]dioxino[2,3-c]pyridin-7-ylmethyl)amino]-5,6,7,8-tetrahydro-2-naphthalenyl}-7-fluoro-2(1H)-quinolinone;
1-{(6R)-6-[(2,3-Dihydro[1,4]dioxino[2,3-c]pyridin-7-ylmethyl)amino]-5,6,7,8-tetrahydro-2-naphthalenyl}-7-fluoro-2(1H)-quinolinone;
4-{6-[(2,3-Dihydro[1,4]dioxino[2,3-c]pyridin-7-ylmethyl)amino]-5,6,7,8-tetrahydro-2-naphthalenyl}-6-(methyloxy)pyrido[2,3-b]pyrazin-3(4H)-one or an enantiomer thereof;
4-{6-[(2,3-Dihydro[1,4]dioxino[2,3-c]pyridin-7-ylmethyl)amino]-5,6,7,8-tetrahydro-2-naphthalenyl}-6-(methyloxy)pyrido[2,3-b]pyrazin-3(4H)-one or an enantiomer thereof;
4-{6-[(6,7-Dihydro[1,4]dioxino[2,3-c]pyridazin-3-ylmethyl)amino]-4,5,6,7-tetrahydro-1,3-benzothiazol-2-yl}-6-(methyloxy)pyrido[2,3-b]pyrazin-3(4H)-one;
6-[({3-[6-(Methyloxy)-3-oxopyrido[2,3-b]pyrazin-4(3H)-yl]-5,6,7,8-tetrahydro-7-quinolinyl}amino)methyl]-2H-pyrido[3,2-b][1,4]oxazin-3(4H)-one;
4-{7-[(6,7-Dihydro[1,4]dioxino[2,3-c]pyridazin-3-ylmethyl)amino]-5,6,7,8-tetrahydro-3-quinolinyl}-6-(methyloxy)pyrido[2,3-b]pyrazin-3 (4H)-one;
4-{7-[(2,3-Dihydro[1,4]dioxino[2,3-c]pyridin-7-ylmethyl)amino]-5,6,7,8-tetrahydro-3-quinolinyl}-6-(methyloxy)pyrido[2,3-b]pyrazin-3 (4H)-one;
Racemic 6-{[({6-[6-(Methyloxy)-3-oxopyrido[2,3-b]pyrazin-4(3H)-yl]-1,2,3,4-tetrahydro-1-naphthalenyl}methyl)amino]methyl}-2H-pyrido[3,2-b][1,4]oxazin-3(4H)-one;
4-(5-{[(2,3-Dihydro[1,4]dioxino[2,3-c]pyridin-7-ylmethyl)amino]-methyl}-5,6,7,8-tetrahydro-2-naphthalenyl)-6-(methyloxy)pyrido[2,3-b]pyrazin-3(4H)-one;
4-(5-{[(6,7-Dihydro[1,4]dioxino[2,3-c]pyridazin-3-ylmethyl)amino]-methyl}-5,6,7,8-tetrahydro-2-naphthalenyl)-6-(methyloxy)pyrido[2,3-b]pyrazin-3(4H)-one;
Cis-6-[({6-Hydroxy-3-[6-(methyloxy)-3-oxopyrido[2,3-b]pyrazin-4(3H)-yl]-5,6,7,8-tetrahydro-7-quinolinyl}amino)methyl]-2H-pyrido[3,2-b][1,4]oxazin-3 (4H)-one;
Cis-4-{7-[(6,7-Dihydro[1,4]dioxino[2,3-c]pyridazin-3-ylmethyl)amino]-6-hydroxy-5,6,7,8-tetrahydro-3-quinolinyl}-6-(methyloxy)pyrido[2,3-b]pyrazin-3(4H)-one;
Cis-4-{7-[(2,3-Dihydro[1,4]dioxino[2,3-c]pyridin-7-ylmethyl)amino]-6-hydroxy-5,6,7,8-tetrahydro-3-quinolinyl}-6-(methyloxy)pyrido[2,3-b]pyrazin-3 (4H)-one;
4-{7-[(2,3-dihydro[1,4]dioxino[2,3-c]pyridin-7-ylmethyl)amino]-5-methyl-6-oxo-5,6,7,8-tetrahydro-1,5-naphthyridin-3-yl}-6-(methyloxy)pyrido[2,3-b]pyrazin-3(4H)-one;
4-(8-{[(2,3-Dihydro[1,4]dioxino[2,3-c]pyridin-7-ylmethyl)amino]methyl}-7,8-dihydro-5H-pyrano[4,3-b]pyridin-3-yl)-6-(methyloxy)pyrido[2,3-b]pyrazin-3(4H)-one;
4-{3-[(2,3-Dihydro[1,4]dioxino[2,3-c]pyridin-7-ylmethyl)amino]-3,4-dihydro-2H-pyrano[3,2-b]pyridin-7-yl}-6-(methyloxy)pyrido[2,3-b]pyrazin-3(4H)-one;
4-{(6R/S,7R/S)-7-[(2,3-Dihydro[1,4]dioxino[2,3-c]pyridin-7-ylmethyl)amino]-6-hydroxy-5,6,7,8-tetrahydro-3-quinolinyl}-6-(methyloxy)pyrido[2,3-b]pyrazin-3(4H)-one;
6-[({3-[6-(Methyloxy)-3-oxopyrido[2,3-b]pyrazin-4(3H)-yl]-5,6,7,8-tetrahydro-7-quinolinyl}amino)methyl]-2H-pyrido[3,2-b][1,4]oxazin-3(4H)-one or an enantiomer thereof;
6-[({3-[6-(Methyloxy)-3-oxopyrido[2,3-b]pyrazin-4(3H)-yl]-5,6,7,8-tetrahydro-7-quinolinyl}amino)methyl]-2H-pyrido[3,2-b][1,4]oxazin-3(4H)-one 2 or an enantiomer thereof;
Cis-6-[({6-Hydroxy-3-[6-(methyloxy)-3-oxopyrido[2,3-b]pyrazin-4(3H)-yl]-5,6,7,8-tetrahydro-7-quinolinyl}amino)methyl]-2H-pyrido[3,2-b][1,4]oxazin-3(4H)-one or an enantiomer thereof;
Cis-6-[({6-Hydroxy-3-[6-(methyloxy)-3-oxopyrido[2,3-b]pyrazin-4(3H)-yl]-5,6,7,8-tetrahydro-7-quinolinyl}amino)methyl]-2H-pyrido[3,2-b][1,4]oxazin-3 (4H)-one or an enantiomer thereof;
Cis-4-{7-[(2,3-Dihydro[1,4]dioxino[2,3-c]pyridin-7-ylmethyl)amino]-6-hydroxy-5,6,7,8-tetrahydro-3-quinolinyl}-6-(methyloxy)pyrido[2,3-b]pyrazin-3(4H)-one or an enantiomer thereof;
Cis-4-{7-[(2,3-Dihydro[1,4]dioxino[2,3-c]pyridin-7-ylmethyl)amino]-6-hydroxy-5,6,7,8-tetrahydro-3-quinolinyl}-6-(methyloxy)pyrido[2,3-b]pyrazin-3(4H)-one or an enantiomer thereof;
4-{7-[(3,4-dihydro-2H-pyrido[3,2-b][1,4]oxazin-6-ylmethyl)amino]-5,6,7,8-tetrahydro-3-quinolinyl}-6-(methyloxy)pyrido[2,3-b]pyrazin-3(4H)-one;
6-[({2-[6-(methyloxy)-3-oxopyrido[2,3-b]pyrazin-4(3H)-yl]-5,6,7,8-tetrahydro-6-quinazolinyl}amino)methyl]-2H-pyrido[3,2-b][1,4]oxazin-3(4H)-one;
4-{6-[(2,3-Dihydro[1,4]dioxino[2,3-c]pyridin-7-ylmethyl)amino]-5,6,7,8-tetrahydro-2-quinazolinyl}-6-(methyloxy)pyrido[2,3-b]pyrazin-3(4H)-one;
6-{[({6-[6-(Methyloxy)-3-oxopyrido[2,3-b]pyrazin-4(3H)-yl]-1,2,3,4-tetrahydro-1-naphthalenyl}methyl)amino]methyl}-2H-pyrido[3,2-b][1,4]oxazin-3(4H)-one or an enantiomer thereof;
6-{[({6-[6-(Methyloxy)-3-oxopyrido[2,3-b]pyrazin-4(3H)-yl]-1,2,3,4-tetrahydro-1-naphthalenyl}methyl)amino]methyl}-2H-pyrido[3,2-b][1,4]oxazin-3(4H)-one or an enantiomer thereof;
2-{[({3-[6-(Methyloxy)-3-oxopyrido[2,3-b]pyrazin-4(3H)-yl]-7,8-dihydro-5H-pyrano[4,3-b]pyridin-8-yl}methyl)amino]methyl}-5,8-dihydropyrido[2,3-d]pyrimidin-7(6H)-one;
6-{[({3-[6-(methyloxy)-3-oxopyrido[2,3-b]pyrazin-4(3H)-yl]-7,8-dihydro-5H-pyrano[4,3-b]pyridin-8-yl}methyl) amino]methyl}-2H-pyrido[3,2-b][1,4]oxazin-3 (4H)-one;
1-{7-[(6,7-Dihydro[1,4]dioxino[2,3-c]pyridazin-3-ylmethyl)amino]-5,6,7,8-tetrahydro-3-quinolinyl}-7-(methyloxy)pyrido[2,3-b]pyrazin-2(1H)-one;
6-[({3-[7-(Methyloxy)-2-oxopyrido[2,3-b]pyrazin-(2H)-yl]-5,6,7,8-tetrahydro-7-quinolinyl}amino)methyl]-2H-pyrido[3,2-b][1,4]oxazin-3(4H)-one;
1-{7-[(2,3-Dihydro[1,4]dioxino[2,3-c]pyridin-7-ylmethyl)amino]-5,6,7,8-tetrahydro-3-quinolinyl}-7-(methyloxy)pyrido[2,3-b]pyrazin-2(1H)-one;
4-{7-[(2,3-Dihydro-1,4-benzodioxin-6-ylmethyl)amino]-5,6,7,8-tetrahydro-3-quinolinyl}-6-(methyloxy)pyrido[2,3-b]pyrazin-3 (4H)-one;
2-[({3-[6-(Methyloxy)-3-oxopyrido[2,3-b]pyrazin-4(3H)-yl]-5,6,7,8-tetrahydro-7-quinolinyl}amino)methyl]-1H-pyrimido[5,4-b][1,4]oxazin-7(6H)-one; and
6-[({7-[6-(Methyloxy)-3-oxopyrido[2,3-b]pyrazin-4(3H)-yl]-3,4-dihydro-2H-pyrano[3,2-b]pyridin-3-yl}amino)methyl]-2H-pyrido[3,2-b][1,4]oxazin-3 (4H)-one;
or a pharmaceutically acceptable salt thereof.

9. A method of treatment of bacterial infections in mammals, particularly in man, which method comprises the administration to a mammal in need of such treatment an effective amount of a compound according to claim 1.

10-12. (canceled)

13. A pharmaceutical composition comprising a compound according to claim 1 and a pharmaceutically acceptable carrier.
